(19) United States
(12) Patent Application Publication
(10) Pub. No.: US 2024/0035101 A1
(43) Pub. Date: Feb. 1, 2024

(54) METHOD FOR SELECTING ANTIGENIC VIRAL SEQUENCES FOR VACCINES AND THERAPEUTICS

(71) Applicant: Wisconsin Alumni Research Foundation, Madison, WI (US)

(72) Inventors: David O'Connor, Madison, WI (US); Thomas Friedrich, Madison, WI (US); Marc Johnson, Columbia, MO (US)

(21) Appl. No.: 18/363,520

(22) Filed: Aug. 1, 2023

Related U.S. Application Data
(60) Provisional application No. 63/394,159, filed on Aug. 1, 2022.

(51) Int. Cl. C12Q 1/70; C12Q 1/6869

(52) U.S. Cl. CPC C12Q 1/701; C12Q 1/6869; C12Q 2600/156; C12Q 1/6804

(57) ABSTRACT

Disclosed herein are methods for identifying antigenic variants from cryptic lineages arising in a virus population and methods of using the identified antigenic variants.

### CROSS-REFERENCE TO RELATED APPLICATIONS

This application claims priority to U.S. Provisional Application No. 63/394,159 filed on Aug. 1, 2022, the contents of which are incorporated by reference in their entireties.

### STATEMENT REGARDING FEDERALLY SPONSORED RESEARCH

This invention was made with government support under DA053893 awarded by the National Institutes of Health and under 75D30121C11060 and 75D30120009870 awarded by the CDC. The government has certain rights in the invention.

### REFERENCE TO AN ELECTRONIC SEQUENCE LISTING

The contents of the electronic sequence listing (960296.04436.xml; Size: 28,077 bytes; and Date of Creation: Aug. 1, 2023) is herein incorporated by reference in its entirety.

### BACKGROUND OF THE INVENTION

Tracking SARS-CoV-2 genetic diversity is critical to monitoring the emergence of novel variants, including those that are resistant to naturally acquired immunity and/or to medical countermeasures including vaccines, monoclonal antibodies, and small molecule inhibitors. Tracking the evolution and spread of such variants is also important for developing and updating protective vaccines. Because SARS-CoV-2 is shed in urine and feces of infected individuals, SARS-CoV-2 RNA can be extracted and quantified from community wastewater to provide estimates of SARS-CoV-2 community prevalence. This approach is especially powerful since it samples large numbers of individuals in targeted sewersheds and can detect viruses shed by individuals whose infections are not necessarily medically attended, such as asymptomatic individuals, those who abstain from testing, or those who test at home.

The continuing evolution of SARS-CoV-2 and the appearance of variants of concern (VOCs), such as the Omicron VOCs, show that the world is continually confronting unexpected, novel variants. Notably, VOCs that have emerged so far have not evolved in a predictable, stepwise fashion from currently dominant variants, but rather have emerged unpredictably. Many VOCs appear to be derived from variants that last circulated many months prior to the VOC's first detection, suggesting that VOCs evolve and emerge from hosts that are not detected even in very intensive surveillance.

Wherever they emerge from, VOCs undermine the utility of medical countermeasures such as vaccines and monoclonal antibodies. Standard methods only identify virus variants that are already in circulation and are not effective at forecasting variants that are likely to arise in the future. New methods that can identify “evolutionarily advanced” variants relative to currently circulating lineages would enable the development of protective vaccines and therapies.

### BRIEF SUMMARY OF THE INVENTION

The present disclosure provides a method for identifying properties of viral variants that are not yet circulating widely but are likely to emerge and spread in the future. While the data presented herein are directed to identification of these viral variants in SARS-CoV-2, similar methods may be suitable for application to other viruses. These cryptic lineages are viral sequences that contain a combination of mutations that are rarely observed in currently circulating viral variants or clinical samples. The method comprises; collecting urine or stool samples from a subject with a prolonged infection or from wastewater from at least one location; extracting RNA from the wastewater samples; sequencing the variable regions of viral RNA from wastewater and identifying cryptic lineages

In particular embodiments, the present disclosure provides a method for identifying cryptic lineages arising in SARS-CoV-2 or Influenza.

In particular embodiments, the region that is sequenced is the SARS-CoV-2 Spike protein and the variable region is the receptor-binding domain (RBD) of the Spike protein. In particular embodiments, the region that is sequenced is the 19 N-terminal amino acids of the Membrane protein of SARS-CoV-2.

In some embodiments the method further comprises generating antibodies capable of recognizing the cryptic lineages. In particular embodiments the antibodies are neutralizing antibodies and block replication of a virus comprising the cryptic lineage or current circulating viruses; further the antibodies are monoclonal antibodies. These antibodies may be tested against both currently circulating viruses and cryptic lineages that may emerge in the future.

In some embodiments, the method further comprises generating a vaccine comprising at least one of the cryptic lineage sequences. In particular, the vaccine may be a mRNA, peptide, or inactivated viral vaccine. The vaccine may comprise one or more than one antigenic variant. These vaccines may be tested against both currently circulating viruses and cryptic lineages that may emerge in the future.

In some embodiments, the method comprises amplifying the variable regions with RT-PCR primers. In some embodiments, the primers amplify at least a 1.5kb region encoding a SARS-CoV-2 Spike protein.

In some embodiments, the method further comprises analyzing the cryptic lineages containing the antigenic variants and determining if the antigenic variants become variants of concern.

In some embodiments, the method comprises using databases with viral sequences from wastewater or databases with sequences from virally infected subjects are used to identify cryptic lineages containing antigenic variants in the virus population.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Novel SARS-CoV-2 lineages from wastewater. A. Schematic of SARS-CoV-2 genome and the regions that are amplified in this embodiment. B. Distribution of SARS-CoV-2 variants based on patient sequences (patient data obtained from GISAID) and wastewater surveillance. Polymorphisms detected from amplicon sequencing that were used to assign sequences to lineages are shown in the legend. The variants detected from the 14 NYC WWTPs were weighted by flowrate to generate a city-wide average distribution. C. Novel lineages detected from WWTPs. Schematic highlights shared sequences identified from WWTP 10, 11, and 3 are shown. The percent of the sequences from each date that contained the indicated polymorphisms is shown below each lineage. The viral genome copies/L corresponding to each date are shown in Table 4 Some sequences have additional polymorphisms not listed. WNY lineage designations are shown for sequences used for tropism and antibody neutralization analysis.

FIG. 2. ACE2 usage by WNY lineages. A Schematic of lineages and pseudovirion production. WNY1=E484A/F486P/S494P/Q498Y/H519N/F572N,

WNY2=Q493K/S494P/Q498Y/H519N/T572N,

WNY3=K417T/K444T/E484A/F590Y/Q498H,

WNY4 =K417T/N439K/K444N/Y449R/L452R/N460K/S477N/Δ484/F486V/S494T/G496V/Q4 98Y/N501T/G504D/505H/H519Q. The indicated mutations were introduced into a codon-optimized SARS-CoV-2 expression construct. These constructs were used to produce lentiviral pseudovirions containing a Gaussia luciferase reporter. Pseudoviruses containing SARS-CoV-2 Spike with N501Y/A570D were used as a control as this is known to be capable of infecting rodent cells. Pseudoviruses were used to transduce 293FT+TMRPSS2 stably transduced with human, mouse, or rat ACE2. The average and standard deviation from three independent experiments is shown in the table (top to bottom in the legend is left to right in the table). A two-way ANOVA revealed significant differences in receptor utilization (F=17.81, DF=3, 74; P<0.0001).

FIG. 3. Antibody resistance to monoclonal neutralizing antibodies and patient plasma. Lentiviral reporter pseudoviruses containing Gaussia luciferase were generated with parent (D614G), WNY1, WNY2, WNY3, or WNY4 Spike proteins. These pseudoviruses were treated with 2-fold dilutions of indicated monoclonal neutralizing antibody or patient serum and used to infect 293FT±TMPRSS2+human ACE2. Gaussia luciferase levels were quantitated approximately 2-3 days of post-transduction. Representative examples of three experiments with monoclonal antibodies performed in triplicate are shown. Infection was normalized to the wells infected with pseudovirus alone. Patient plasma Neutralization IC50 titers were calculated using nonlinear regression (Inhibitor vs. normalized response—variable slope) in GraphPad Prism 9.0. The number indicates the mean fold of reduction in IC50 and SD. Wilcoxon matched-pairs signed rank tests, a two-tailed test, were performed for paired comparisons with significance levels as follows: WNY3 patient p=0.0049, WNY4 patient p=0.001, and WN V4 vaccinated=0.0068.

FIG. 4. Viral RNA concentration (SARS-CoV-2 N1 copies per L) at each sampling date is plotted against NYC confirmed clinical cases (7-day average) over the sampling period Jan. 1 to Jun. 28, 2021.

FIG. 5. RBD amplification. A. Schematic of regions targeted by the RBI) and S1 primer sets. Overview of the SARS-COV-2 Spike RBD lineages identified in B. the MO33 sewershed and C. the MO45 sewershed. Each row represents a unique lineage and each column is an amino acid position in the Spike protein (left). Amino acid changes similar to (green boxes) or identical to (orange boxes) changes in Omicron (BA.1, BA.2 or BA.5) are indicated. Synonymous changes (syn) are indicated in gray. The major US VOCs (Alpha, Beta, Gamma, BA.1, BA.2, and BA.5) are indicated. The heatmap (right) illustrates lineage (row) detection by date (column), colored by the logic percent relative abundance of that lineage.

FIG. 6. NY3 and NY 1.4 .RBD amplifications. Overview of the SAILS-COV-2 Spike RBD lineages identified from the A. NY3 and B. NY14 sewersheds. Amino acid changes similar to (green boxes) or identical to (orange boxes) changes in Omicron (BA.1, BA.2 or BA.5) are indicated. Synonymous changes (syn) are indicated in gray. The major US VOCs (Alpha, Beta, Gamma, BA.1, BA.2, and BA.5) are indicated. The heatmap (tight) illustrates lineage (row) detection by date (column), colored by the log₁₀ percent relative abundance of that lineage.

FIG. 7. NY10 RBD amplifications. Overview of the SARS-COV-2 Spike RBD lineages identified from the NY10 sewershed. Amino acid changes similar to (green boxes) or identical to (orange boxes) changes in Omicron (BA.1, BA.2 or BA.5) are indicated. Synonymous changes (syn) are indicated in gray. The major VOCs during this time period (Alpha, Beta, Gamma, BA.1 and BA.2) are indicated. The heatmap (right) illustrates lineage (row) detection by date (column), colored by the log₁₀ percent relative abundance of that lineage.

FIG. 8. NY11 and NY2 RBD amplifications. Overview of the SARS-COV-2 Spike RBD lineages identified from the NY11 and NY2 sewershed. Lineages designated A and B belong to two lineages groups that appear unrelated. Amino acid changes similar to (green boxes) or identical to (orange boxes) changes in Omicron (BA.1., BA.2 or BA.5) are indicated. Synonymous changes (syn) are indicated in gray. The major VOCs during this time period (Alpha, Beta, Gamma, BA.1 and BA.2) are indicated. The heatmap (right) illustrates lineage (row) detection by date (column), colored by the logo percent relative abundance of that lineage. Lineage detected in both sewersheds indicated with an asterisk.

FIG. 9. NY13 RBD amplifications. Overview of the SARS-COV-2 Spike RBD lineages identified from the NY13 sewershed. Amino acid changes similar to (green boxes) or identical to (orange boxes) changes in Omicron (BA.1, BA.2 or BA.5) are indicated. Synonymous changes (syn) are indicated in gray. The major VOCs during this time period (Alpha, Beta, Gamma, BA.1 and BA.2) are indicated, The heatmap (right) illustrates lineage (row) detection by date (column), colored by the log₁₀ percent relative abundance of that lineage.

FIG. 10. Overview of the SARS-COV-2 Spike RBD lineages identified from the California sewershed. Amino acid changes similar to (green boxes) or identical to (orange boxes) changes in Omicron (BA.1, BA.2 or BA.5) are indicated. Synonymous changes (syn) are indicated in gray. The major VOCs during this time period (Alpha, Beta, Gamma, BA.1 and BA.2) are indicated. The heatmap (tight) illustrates lineage (row) detection by date (column), colored by the log₁₀ percent relative abundance of that lineage.

FIG. 11. S1 amplifications. A. Overview of the SARS-COV-2 Spike S1 lineages in the Alpha, Delta, Omicron VOCs and six of the sewersheds with cryptic lineages. S1 amplifications were sequenced by subcloning (SC) and Sanger sequencing or were sequenced using a PacBio (PB) deep sequencing. B. Plot of the number of synonymous and non-synonymous changes in the S1 sequences shown.

FIG. 12, Polymorphisms from wastewater genomes. Shown are all mutations present in at least three of the whole genome sequences from NYC listed in Table 8 and their corresponding amino acid changes. First column lists the prevalence of each mutation among all patients samples collected in June 2021 from New York. Each other column lists the prevalence of each mutation in each of the genome sequences.

FIG. 13, Sequence of nt 14408 from NYC wastewater. NY13 and NY11 SEQ ID NO: 29, NY1.0 SEQ ID NO: 30

FIG. 14. Tracking the source of the cryptic SARS-CoV-2 lineage. (a) The Wisconsin Lineage was first detected at the publicly owned treatment works (POTW) facility from one of the five district lines that serve the POTW sewershed. Continued wastewater sampling at interceptor lines that serve the positive district line isolated the lineage's source to a single sub-district (Sub-District 5). Further sampling of maintenance holes within Sub District 5 pointed to a single place of business as the Wisconsin Lineage's source. Sampling at the facility pinpointed a collecting line (Facility Line B) servicing 6 toilets used by facility employees. (b) SARS-CoV-2 concentrations (on a log₁₀ scale) detected in five sampling areas show extremely high levels of SARS-CoV-2 in wastewater from Facility Line B. The Wisconsin Lineage's percent (B.1.234%) contribution to the SARS-CoV-2 levels (estimated by Freyja) at each sampling level is shown in tan. (c) SARS-CoV-2 concentrations throughout 2022. for the Main Plant (POTW) and Sub-District 5 are shown as a blue line. The percent contribution of the Wisconsin Lineage (B.1.234 proportion) is shown as tan bars, depicting the continued detection of the cryptic virus at both sampling levels for most of 2022. Higher B.1.234 proportions were seen in Sub-District 5 than in the Main Plant (POTW), corresponding to its closer proximity to the Wisconsin Lineage's source.

FIG. 15. Representative haplotypes of Wisconsin Lineage sequences. (a) The SARS-CoV-2 spike RBD was amplified from Sub-District 5 wastewater samples using primers designed to exclude Omicron lineages. A single amplicon, spanning Spike amino acid residues 377-606, was sequenced, and the amino acid changes (relative to reference sequence MN908947.3) found in Omicron lineages are shown in blue. (b) Representative haplotypes are displayed, each of which represented at least 25% of the total sequences in at least one sample. Green boxes indicate amino acid sites that are also altered in Omicron (BA.1 or BA.2), blue boxes indicate amino acid sites that have identical mutations to Omicron lineages, and tan boxes indicate amino acid sites that are altered in the Wisconsin Lineage and not in major Omicron lineages. A indicates an in-frame amino acid deletion.

FIG. 16. Prevalence of key cryptic lineage mutations in global sequences. The global proportions of sequences uploaded to NCBI GenBank for key mutations in the spike gene of the Wisconsin wastewater lineage are plotted over time. The spike mutations R346T, V445P, and N460K were all detected in the Wisconsin cryptic lineage months before becoming predominant in global sequences. The Wisconsin Lineage also harbored F486A from the time of initial detection in January 2022. Two other substitutions at spike amino acid residue 486 have since become dominant in global sequences (dotted lines). Searching for the global proportion of sequences was done in coy-SPECTRUM.³⁰

FIG. 17. Diversity analysis of wastewater genomic sequences from all Facility Line B time points. (a) Root-to-tip regression analysis distance) of B.1.234 sequences via TreeTime based on a maximum likelihood phylogenetic tree inferred with iqtree (not shown) and aligned to the MN908947.3 reference sequence. All sequences were obtained from GenBank and can be accessed using the accession numbers available on the GitHub repository accompanying this manuscript. (b) The enumeration of intra-host single nucleotide polymorphisms (iSNVs; y-axis) for the wastewater timepoints for each mutation type following alignment to the reference genome MN908947.3 (colored as in panel a). Mutations were classified as nonsynonymous (Non-syn), synonymous (Syn), insertions-deletions (Indels), or others (including nonsense and frameshift mutations outside of coding regions). (c) The number of nucleotide transitions and transversions from all timepoints. The 95% confidence intervals were obtained from the relative risk (RR) of every nucleotide substitution (i.e. RR=‘A>C’/‘C>A’). (d) We estimated genetic diversity within each sample using the summary statistics πN, which quantifies pairwise nonsynonymous differences per nonsynonymous site (darker bars), and πS, which quantifies pairwise synonymous differences per synonymous site (lighter bars), for each SARS-CoV-2 gene. The 95% confidence intervals were obtained using a binomial probability distribution. A Mann-Whitney two-sided test was applied to test the difference between πN and πS on each gene (red asterisk). A one-sided test was used to test for an enrichment of the πN value of spike against the πN value on the other genes (black asterisks). (e) The divergence (:Hamming distance; y-axis) between B.1,234 isolates from panel (a) and the MN908947.3 reference sequence over a sliding window of 36 days (x-axis) compared to the Wisconsin Lineage isolates. With the exception of the Wisconsin Lineage, data are only plotted when windows contain at least two B.1.234 sequences.

FIG. 18. All variants above 10% frequency in Wisconsin Lineage RBD sequences from Sub-District 5 organized by haplotype.

FIG. 19. Radial phylogenetic tree generated by Nextclade.1 Consensus fasta files generated for each sequence replicate of Facility Line B samples are shown. Although differences exist between some replicate sequences, the Wisconsin Lineage greatly diverges from its B.1.234 backbone and is similarly divergent to Omicron lineages.

FIG. 20. Ohio, Wisconsin, and UK EACL/EAPL genome sequences are about as divergent from the original SARS-CoV-2 as Omicron viruses (clades 21M,K,L, 22*, and 23*).

FIG. 21. Convergent Spike amino acid variants in the Wisconsin, Ohio, and UK EACL/EAPL.

### DETAILED DESCRIPTION OF THE INVENTION

The present disclosure describes methods for identifying mutations encoding antigenic variants arising in a virus population in a forward-looking manner.

SARS-CoV-2 is shed in feces and urine of infected individuals, and SARS-CoV-2 RNA can be extracted and quantified from community wastewater to provide estimates of SARS-CoV-2 community prevalence. This viral RNA (vRNA) can also be sequenced to profile the evolution and spread of SARS-CoV-2 variants. The continuing evolution of SARS-CoV-2 and the appearance of variants of concern (VOCs), such as the Omicron VOC, highlight the importance of maintaining a vigilant watch for the emergence of unexpected, novel variants. The fact that the origins and early spread of the Alpha and Omicron VOCs were not directly observed strongly motivates efforts to detect novel variants as quickly as possible, in order to forecast future viral evolution that could undermine the efficacy of natural or vaccine-induced immunity, and/or other countermeasures such as monoclonal antibodies. Described herein, the inventors demonstrate a novel method for detecting divergent viral sequences that encode amino acid changes that are likely to eventually emerge in widely circulating viruses. This method utilizes wastewater sampling and sequencing to detect divergent sequences that are not captured in typical surveillance testing of individuals. These methods allow for the detection of patterns of mutations that are likely to become more prevalent or properties of viral variants that are not yet circulating widely but are likely to emerge and spread in the future.

Viruses mutate as they replicate and spread in a population creating viral variants. The more a virus replicates, the more opportunities for mutations to arise. A viral variant may contain one or more mutations. In some cases, a viral variant may affect the virus's ability to spread, cause disease, or respond to current medial countermeasures, resulting in a competitive advantage over the other lineages of the virus. These are known as variants of concern (VOC), or variants of interest (VOI). In order to monitor for VOC or VOI, viruses are typically sequenced from clinical samples isolated from currently infected individuals. These samples are surveyed to identify which variant of a virus is currently circulating in a population. However, this type of viral surveillance fails to detect viral variants in asymptomatic individuals, individuals with prolonged infections, individuals with mild symptoms that do not report to a clinical setting where novel variants are emerging.

Some embodiments provide a method for identifying cryptic lineages, a specific type of sequence observed in wastewater which comprise patterns or collections of mutation that are rarely observed in contemporaneous clinical samples. Broadly, “variants” are microbes bearing one or more changes in nucleotide sequence relative to some reference sequence. The change may be benign, pathogenic or of unknown significance. A variant may be a pathogen with a particular set of mutations. Specifically, an antigenic variant is a nucleotide change resulting in a change in the amino acid sequence of a protein encoded by an infectious agent, which disrupts the ability of immune responses to recognize the variant. Some antigenic variants allow the infectious agent to re-infect previously infected hosts. Antigenic variants can also reduce the efficacy of countermeasures such as therapeutic monoclonal antibodies. VOCs may also harbor other mutations that reduce the efficacy of countermeasures such as antiviral medications. The methods described herein allow for the identification of antigenic variants in a virus population. The methods may be particularly suited for viruses that are known to be detectable in wastewater and have high mutation rates that necessitate frequent vaccine updates. This includes viruses such as the influenza virus and severe acute respiratory syndrome (SARS)-coronavirus (CoV)-2. Respiratory syncytial virus (RSV) is detectable in wastewater and may also begin to evolve more rapidly under immune pressure following the approval of novel vaccines. Similarly, mpox and Human Immunodeficiency Virus (HIV) can also be detected in wastewater. The methods described herein may be further suited for viruses that typically cause short, acute infections but may occasionally establish prolonged infection. The methods may also be suited for virus populations which cause a prolonged infection such as polio virus. The methods may be suitable for any virus for which surveying antigenic variants may inform antigen targets in vaccine development.

The methods described herein allow for the identification of antigenic variants in a virus population, including in the SARS-CoV-2 virus. The virus causes COVID-19, a severe respiratory disease that rapidly spread across China beginning in late 2019 and was declared a world-wide pandemic in early 2020. Like other CoVs, the SARS-CoV-2 spike (S) protein is assumed to be the major target for neutralizing antibodies. To enable entry of host cells, the SARS-CoV-2 S protein binds to the receptor, angiotensin-converting enzyme 2 (ACE2), through its receptor binding domain (RBD). The RBDs for other CoVs are immunogenic and a major neutralizing determinant. In the long term, it is likely that SARS-CoV-2 will continue circulating in humans and causing disease. Thus, development of additional safe and effective vaccines against the virus is a significant priority. In order to remain effective, these vaccines will almost certainly need to continually adapt to changes in circulating variants as SARS-CoV-2 continues its antigenic evolution. Additionally, continued SARS-CoV-2 evolution is likely to impact the efficacy of countermeasures like therapeutic monoclonal antibodies and antivirals, which have been critical in reducing morbidity and mortality. Therefore, approaches that can accurately forecast the direction of SARS-CoV-2 antigenic evolution will be critical for preparing vaccine updates, predicting the efficacy of countermeasures, and other applications.

In specific embodiments, the methods include identify antigenic variants in the spike protein, including the receptor binding domain (RBD) of the spike protein and antigenic variants in the membrane protein, including in the first 19 N-terminal amino acids of the membrane protein of SARS-CoV-2. Other antigenic variants that are selected by the human immune system, including antibodies and T cell responses, can also be identified by the methods.

The method of the present disclosure includes collecting wastewater samples. Wastewater is used water from any combination of domestic, industrial, commercial or agricultural activities, surface runoff/storm water, and any sewer inflow or sewer infiltration. Wastewater may also be called sewage, raw wastewater or raw sewage. Sewage is wastewater that is produced by a community of people. Samples for the methods described herein may be collected from wastewater, including from a sewershed, sewage lift-station, manhole, facility sewer line access point, individual toilets and household waste sources or any other sewer access point. Samples collected for use in the method described herein may include any material found in wastewater, including but not limited to feces, urine, and any other biological material typically found in wastewater. A sewershed is an area where water drains into a single point of the sewer system. As described herein, the samples may be taken from 1 single wastewater location, or from multiple, including two, three, four, five, six, seven or more locations. Samples may be taken one or more times from a site. When more than one sample is collected from a site, the time between each sample collection may be hours, days, weeks, months or years apart. For example, a sample may be collected from a site, followed by another sample collected from the same site one week later, one month later, two months later, six months later, a year later, or more than a year later, and any amount of time in between for any number of sample collections.

In some embodiments, the method may comprises tracking the source of the cryptic SARS-CoV-2 lineage. As described in Example 3, successive sampling at various points in a sewershed, for example publicly owned treatment works facility, district lines, sub-district lines, maintenance holes, and facility lines allows cryptic lineages to be traced to a specific origin.

In some embodiments RNA is extracted from wastewater samples. RNA may also be extracted from feces (also known as stool or excrement) or urine samples. RNA extraction is the purification of RNA from biological samples. RNA may be extracted by any means known in the art. Common RNA extraction techniques include organic extraction, such as phenol-Guanidine Isothiocyanate (GITC)-based solutions, silica-membrane based spin column technology, and paramagnetic particle technology. In some embodiments, wastewater may be concentrated prior to isolation of RNA.

In some embodiments, isolated RNA may be quantified by reverse transcription polymerase chain reaction (RT-PCR). RT PCR is a technique combining reverse transcription of RNA into DNA (called complementary DNA or cDNA) and amplification of specific DNA targets using polymerase chain reaction (PCR). RT-PCR can be used to measure the amount of a specific RNA. RT-PCR requires the use of site-specific primers. PCR primers are single-stranded oligonucleotides. Two primers are used in each PCR reaction, and they are designed so that they flank the target region that should be copied. That is, they are given sequences that will make them bind to opposite strands of the template DNA, just at the edges of the region to be copied. The primers bind to the template by complementary base pairing. Sequencing adapters and molecular barcodes may additionally be used. Adapter sequences are short oligonucleotides used to be ligated to the ends of DNA fragments of interest, so that they can be combined with primers for amplification. Sequencing adapter sequences are known in the art. In some embodiments, the PCR primer may comprise SEQ ID NOs: 6, 7, 8, 9, 14, 15, 16, 17, 18, 20, 21, 22, 23, 24, 26, 27 and 28. In some embodiments, the RT-PCR primers amplify an antigen binding region of a virus. In some embodiments, the RT-PCR primers amplify a spike protein, a membrane protein or a receptor binding domain of the spike protein of SARA-CoV-2. In some embodiments, the RT-PCR primers amplify at least a 1.5 kb region encoding a SARS-CoV-2 spike protein.

The term “amplifying” as used herein generally refers to the production of a plurality of nucleic acid molecules from a target nucleic acid wherein primers hybridize to specific sites on the target nucleic acid molecules in order to provide an initiation site for extension by a polymerase. Amplification can be carried out by any method generally known in the art, such as, but not limited to: standard PCR, long PCR, hot start PCR, qPCR, RT-PCR and Isothermal Amplification. Other amplification reactions comprise, among others, the Ligase Chain Reaction, Polymerase Ligase Chain Reaction, Gap-LCR, Repair Chain Reaction, 3 SR, NASBA, Strand Displacement Amplification (SDA), Transcription Mediated Amplification (TMA), and Q3-amplification.

In some embodiments, the amplified variable region is sequenced. Sequencing determines the sequence of nucleic acids or amino acids. Sequencing may be done by any means known in the art. By way of example, but not by way of limitation, types of sequencing include, the Sanger method, high throughput methods, next generation sequencing methods, sequencing by synthesis and reversible dye-terminators and single molecule, real time sequencing. In some embodiments, SARS-CoV-2 whole genome sequencing is performed. In some embodiments, the variable region comprises the antigen binding region of a virus. In some embodiments, the variable region comprises the spike region, the receptor-binding domain (RBD) of the spike domain and/or the membrane domain of SARS-CoV-2.

In some embodiments, the method provides from collecting a urine or stool sample from a subject with a prolonged viral infection. The subject may be symptomatic or asymptomatic of viral infection. In some embodiments, the subject may have a prolonged infection with SARS-CoV-19, the subject may have Long COVID, long-haul COVID, post-COVID-19 or chronic COVID. In some embodiments, the subject may be immunosuppressed, immunocompromised or have a immunodeficiency. In some embodiments, the subject may not be aware they have a viral infection prior to wastewater, urine or stool sampling.

In some embodiments, genetic sequencing databases may be utilized to identify cryptic lineages. By way of example, and not limitation, these databases may include National Center for Biotechnology Information (NCBI) GenBank, GenomeTrakr, NCBI Sequence Read Archive (NCBI SRA) and Global Initiative on Sharing All Influenza Data (GISAID) databases. Consensus sequences from databases such as NCBI Genbank or GISAID, or datasets containing sequencing reads from databases such as NCBI SRA may be examined for signatures of cryptic lineages. These signatures include amino acid changes that have been found to be enriched in cryptic lineages. It may also include variants that have evidence of intramolecular recombination during prolonged infection. It may also include sequences in databases that are anachronistic, bearing sequence signatures that are rare or extinct among sequences at the time of wastewater collection but that were common at one or more times in the past.

Sequences generated by the methods described herein, and sequences identified in a database will be screened for reproducible lineages that do not match the known circulating lineages. Each sequence with a unique combination of amino acid changes is referred to as a lineage, and combinations of lineages that all have specific amino acid changes in common as lineage classes. Amino acid combinations identified that have not been seen previously from patients are referred to as cryptic lineages. Cryptic lineages are those sequences identified in at least two independent samples. Cryptic lineages may comprise one or more non-synonymous substitutions, insertions and/or deletions. For example, as described in Example 2, MO33 comprised 4-5 RBD amino acid changes and was consistently detected at low relative abundances from March 15 to the end of April 2021 and the NY10 lineage was first detected in April 2021 and comprised 4-5 RBD amino acid changes, but several months later comprised 6-8 RBD amino acid changes, Cryptic lineages may be detected then not detected, and then detected again some time later. For example, as described in Example 2, MO45 lineage was detected in June 2021, then not seen again until February 2022, Cryptic lineages can arise from prolong infection with a virus. Thus, cryptic lineages may also comprise changes in the viral genome that are typically reflective of those that were common at the time the individual was originally infected with the virus.

In some embodiments, the methods described herein may further comprise identifying viral sequence changes which persist and eventually emerge in widely circulating virus variants of concern or variants of interest. The source of cryptic lineages may be from an under-sampled animal reservoir or from persistently infected humans. As described in Example 3, samples collected from a single facility contained a combination of fixed nucleotide substitutions. These divergent sequences contain amino acid changes that eventually emerged in circulating viruses. In this way, the method described herein can inform viral vaccine target development.

In some embodiments the antigenic variants or cryptic lineages identified with the methods described herein arise under positive selection. Positive selection is the process by which new advantageous genetic variants arise in a population. Positive selection causes a shift to the advantageous genetic variant over time because of a benefit to the organism, often survival or reproduction. In some embodiments, the methods described herein identify a positively selected antigenic variant. As demonstrated in Example 3 the methods described herein utilize repeat sampling of wastewater to capture and monitor viral sequences over time. This method can identify persistent antigenic variants from a narrow location which increase in abundance under positive selection.

In some embodiments, the method includes testing recombinant viruses comprising potential antigenic variants from cryptic lineages in an antibody neutralization assay. A neutralizing antibody is an antibody that prevents infection of a cell by a virus. Neutralization renders the virus particle no longer infectious. An antibody neutralization assay determines the functional ability of antibodies to prevent infection by SARS-CoV-2. Further, in some embodiments, antibodies may be generated that recognize the antigenic variants. In particular embodiments, the generated antibodies may block the replication of a virus comprising the antigenic variant. The antibodies may be monoclonal. The method may further comprise generating antibodies that recognize mutations found in cryptic. lineages as well as those found in known VOC.

In some aspects of the present disclosure, a vaccine may be generated. The vaccine may comprise at least one of the antigenic variants found in a cryptic lineage or at least one mutation identified in a cryptic lineage. The vaccine may further comprise two, three, four, five, six or more antigenic variants or cryptic lineage mutations. The vaccine may also comprise other antigenic variants, for example those found in a currently circulating viral population, VOC or VOI, or mutations that stabilize the spike protein. As such a vaccine immunogen generated from the methods described herein may not necessarily be identical to any one cryptic lineage but may include one or more mutations from a cryptic lineage and other mutations in known variants or for the benefit of the vaccine function. The vaccine may be a mRNA, peptide, viral vectored, live attenuated, or inactivated viral vaccine.

Vaccine compositions including the cryptic lineages described herein are also provided. In some embodiments, the vaccine composition includes SARS-CoV-2 cryptic lineages described herein. As used herein “vaccine” refers to a composition that includes an antigen. Vaccine may also include a biological preparation that improves immunity or the immune response to a particular disease. A vaccine may typically contain an agent, referred to as an antigen, that resembles or is a part of a disease-causing microorganism, in this case a virus, for example SARS-CoV-2, and the agent may be nucleic acids that are homologous to a portion of the virus, or often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the intramuscular, intravenous, subcutaneous, oral, oronasal, or intranasal routes of administration.

The vaccine compositions described herein also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfate, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the cryptic lineages described herein.

Suitable adjuvants are known in the art and include, but are not limited to, threonyl muramyl dipeptide (MDP) (Byars et al., 1987), Ribi adjuvant system components (Corixa Corp., Seattle, Wash.) such as the cell wall skeleton (CWS) component, Freund's complete adjuvants, Freund's incomplete adjuvants, bacterial lipopolysaccharide (LPS; e.g., from E. coli), or a combination thereof. A variety of other well-known adjuvants may also be used with the methods and vaccines of the invention, such as aluminum hydroxide, saponin, amorphous aluminum hydroxyphosphate sulfate (AAHS), aluminum hydroxide, aluminum phosphate, potassium aluminum sulfate (Alum), and combinations thereof. Cytokines (.gamma.-IFN, GM-CSF, CSF, etc.), lymphokines, and interleukins (IL-1, IL-2, IL-3, IL-4, IL-5, IL-6, IL-7, IL-8. IL-9, IL-10, IL-11, IL-12, IL-13, IL-14, IL-15, IL-16, IL-17, 11-18, 11-19, IL-20, IL-21, and 11-22) have also been used as adjuvants and/or supplements within vaccine compositions and are contemplated to be within the scope of the present invention. For example, one or more different cytokines and/or lymphokines can be included in a composition comprising one or more peptides or a vaccine of the invention. In a preferred embodiment, the adjuvant is an aluminum salt, AS04, MF59, ASO1B, CpG 1018, or another adjuvant that is considered to be safe for use in humans by the Centers for Disease Control and Prevention.

To aid in administration, vaccines may be mixed with a suitable carrier or diluent such as water, oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final vaccine formulation.

The remainder of the vaccine formulation may be an acceptable diluent, to 100%, including water. The vaccine formulation may also be formulated as part of a water-in-oil, or oil-in-water emulsion.

The vaccine formulation may be separated into vials or other suitable containers. The vaccine formulation herein described may then be packaged in individual or multi-dose ampoules or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user and administered to a patient.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Typically, percentage of sequence identity usually refers to the ratio of the number of matching residues to the total length of the alignment, while percent similarity includes “similar” residues, for example leucine and valine, as well as identical ones.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 75%. Preferred percent identity of polypeptides can be any integer from 75% to 100%. More preferred embodiments include at least 75%, 80%, 85%, 86%, 87%, 88%, 89%, 90%, 91%, 92%, 93%, 94%, 95%, 96%, 97%, 98%, 98.7%, or 99%.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Example 1: Tracking Cryptic SARS-CoV-2 Lineages Detected in NYC Wastewater

Reference to Smyth, D. S., Trujillo, M., Gregory, D. A. et al. Tracking cryptic SARS-CoV-2 lineages detected in NYC wastewater. Nat Commun 13, 635 (2022). is hereby incorporated in its entirety.

SARS-CoV-2 is shed in feces and can be detected by RT-qPCR in wastewater correlating to caseloads in sewersheds^{1,2,3} Consequently, municipalities and public health organizations have employed wastewater surveillance as a public health tool to make informed decisions about COVID-1.9 interventions^{2,4}. However, the standard application of RT-qPCR does not provide genotype information and consequently cannot be used to monitor SARS-CoV-2 evolution and track variants of concern. Some researchers have applied, with mixed success, high-throughput sequencing strategies to total RNA extracted from wastewater. Often, coverage across the SARS-CoV-2 genome is uneven and epidemiologically informative regions can have low coverage^{5,6}. Additionally, because wastewater samples contain an amalgamation of lineages circulating in the sewershed, it is not possible to reconstruct individual genomes using standard methods. Because of these difficulties, some researchers are using a strategy that employs the amplification and sequencing of small, specific regions of the SARS-CoV-2 genome, i.e., targeted sequencing 7,8 . Targeted sequencing can provide high coverage of epidemiologically informative regions of the genome and importantly, can reveal which polymorphisms are linked, thus allowing SARS-CoV-2 variants of concern (VOC) in communities to be tracked.

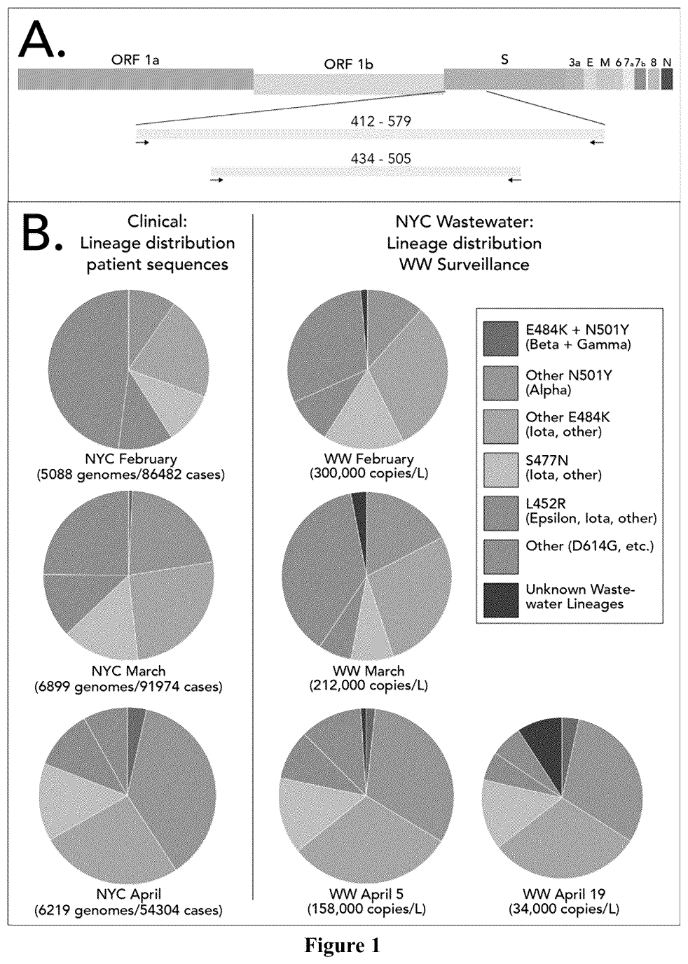

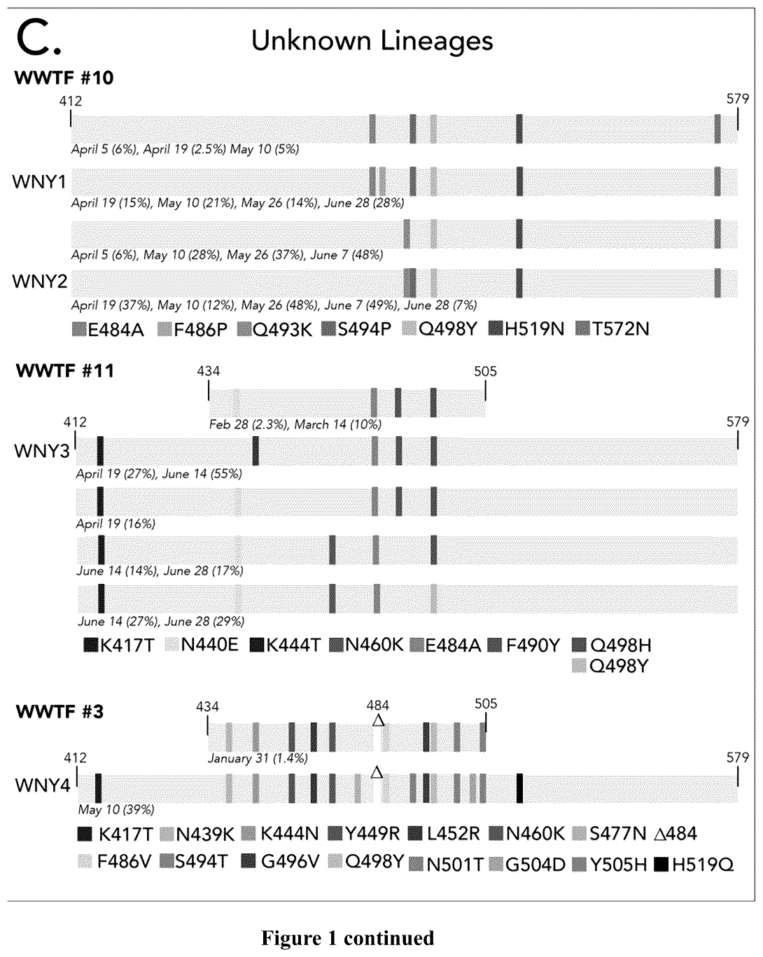

Since January of 2021, we sequenced SARS-CoV-2 RNA isolated from the raw influent from all 14 NYC WWTPs approximately twice per month'. Initially, we used an iSeq instrument to sequence a PCR-amplified region of the SARS-CoV-2 spike protein gene. This region spanned spike protein amino acid residues 434-505, which includes the receptor binding domain (RBD) (FIG. 1A). Beginning in April 2021, we switched to using a MiSeq instrument, which allowed us to sequence a larger amplicon that included amino acid residues 412-579. While no samples were analyzed with both the iSeq and MiSeq, the same constellations of mutations were consistently observed in the respective sewersheds regardless of the instrument used. These regions contain loci that are significant in SARS-CoV-2 receptor tropism and immune evasion, and contain multiple polymorphisms found in many VOCs^{9,10}.

### Results and Discussion

### Identification of Novel Cryptic Sewershed-Specific Lineages

Our analysis pipeline, which uses the tool SAM Refiner to report polymorphisms and remove artificial chimeric sequences, allowed us to determine the frequency of each polymorphism and more importantly, elucidate which polymorphisms were derived from the same RNA sequence⁸. Freebayes and IGV were used to validate the reported polymorphisms (see “Methods” section). Using this approach, we were able to classify suites of mutations found in the RBD amplicons as consistent with Pango lineages B.1.1.7 (Alpha), B.1.351 (Beta), B.1.427/429 (Epsilon), B.1.526 (Iota), B.1.617 (Delta and Kappa), and. P.1 (Gamma). Importantly, the distributions and trends in viral lineages from wastewater were consistent with patient derived sequences from NYC submitted to the GISAID EpiCoV database (hereafter, GISAID; https://www.gisaid.org/) (FIG. 113 and Table 5 FIG. 4). For example, between February and April, wastewater and patient sequencing both revealed a notable increase in sequences assigned to the Alpha lineage and a corresponding decrease in sequences that did not belong to any of the VOC lineages.

TABLE 5
Mutation frequencies with respect to Wuhan reference
sequence (NC_045512.2) in iSeq sequenced samples
assayed between 2021 Jan. 31 and 2021 Mar. 14.

| Mutation | Frequency |
|---|---|
| A435S | 1 |
| A475A | 10 |
| A475S | 2 |
| A475V | 1 |
| C435T | 1 |
| C480C | 1 |
| C488C | 3 |
| E465V | 1 |
| E471G | 2 |
| E484A | 7 |
| E484K | 33 |
| E484Q | 1 |
| F464L | 1 |
| F486I | 1 |
| F486S | 1 |
| F490F | 2 |
| F490L | 1 |
| F490S | 2 |
| F490Y | 4 |
| F497S | 1 |
| G446V | 1 |
| G476D | 1 |
| G482G | 2 |
| G482N | 3 |
| G485G | 2 |
| G485S | 3 |
| G502G | 3 |
| I434L | 3 |
| I468I | 2 |
| I468T | 1 |
| K444K | 1 |
| K444N | 1 |
| K444T | 1 |
| K458E | 1 |
| K458N | 1 |
| K458R | 1 |
| L441F | 1 |
| L452R | 35 |
| L452Z | 2 |
| L455F | 1 |
| L455M | 1 |
| L455V | 1 |
| L461I | 1 |
| L461P | 1 |
| L462R | 1 |
| L492S | 1 |
| N427E | 14 |
| N428E | 14 |
| N437N | 2 |
| N440E | 13 |
| N440S | 1 |
| N440Y | 1 |
| N448S | 1 |
| N450D | 1 |
| N450N | 1 |
| N460L | 1 |
| N460N | 1 |
| N487D | 1 |
| N501S | 6 |
| N501T | 11 |
| N501Y | 33 |
| P463P | 4 |
| P479P | 1 |
| P491P | 1 |
| P499H | 1 |
| P499P | 1 |
| Q474Q | 1 |
| Q493L | 1 |
| Q498* | 6 |
| Q498H | 9 |
| Q506* | 3 |
| Q506E | 25 |
| Q507E | 5 |
| S438T | 1 |
| S443P | 1 |
| S459T | 1 |
| S469L | 1 |
| S469P | 1 |
| S469S | 1 |
| S477G | 2 |
| S477N | 28 |
| S494L | 1 |
| S494P | 30 |
| T470K | 2 |
| T478K | 11 |
| V433A | 1 |
| V433F | 1 |
| V445A | 1 |
| V483F | 3 |
| W436R | 1 |
| Y449R | 4 |
| Y449Y | 1 |
| Y473H | 1 |
| Y495H | 1 |
| Y495Y | 1 |
| Deletion and Frameshift | 18 |
| | 420 |

In addition to well-recognized lineages, WWTPs 3, 10, and 11 contained RBD sequences with consistent constellations of polymorphisms detected over several months that did not match lineages reported in GISAID (FIG. 1C). Herein we refer to these constellations of linked mutations in the RBI) sequences as lineages (meaning that they are of common descent), although without having the complete genome sequence we cannot say whether these were derived from a single lineage or multiple lineages with the same RBD sequence. These cryptic lineages were not static, as several of them appeared to acquire additional polymorphisms over the period of sampling. For example, one of the lineages from WWTF 10 added the polymorphism F486P at later sampling dates (FIG. 1C).

The cryptic lineages all remained relatively geographically constrained. The lineages from WWTP 3 and WWTP 10 were only observed from those locations during this sampling period. Sequences resembling the lineages from WWTP 11 were occasionally seen in neighboring sewersheds. Four of the anomalous lineages, designated WNY1, WNY2, WNY3, and WNY4, were selected for further study. Each of these lineages contained at least five polymorphisms; the most divergent was WNY4, which contained 16 amino acid changes in its RBD including a deletion at position 484. We note that WNY4 and the Omicron VOC possess mutations at the overlapping residues in the RBD, including K417, S477, T478, E484, G496, Q498, N501, and Y505. Polymorphisms at several of these positions have been reported to evade neutralization by particular antibodies^{9,11,12,13,14}.

Interestingly, all four WNY lineages contained a polymorphism at spike protein residue 498 (Q498H or Q498Y). As of Nov. 30, 2021, there were only 35 SARS-CoV-2 sequences in GISAID that contained the polymorphism Q498H, (eight in the USA), and none that contained Q498Y. However, both of these polymorphisms have been associated with host range expansion of SARS-CoV-2 into rodents^{15,16,17,} which are generally resistant to the parent SARS-CoV-2 lineage^{18,19,20}. Notably, as the concentration of SARS-CoV-2 genetic material from NYC wastewater decreased along with the decrease in COVID patients, the fraction of the total sequences from these lineages has proportionally increased (FIG. 1C and FIG. 4). By May and June, these lineages often represented the majority of sequences recovered from some sewersheds. For instance, on June 7 the sequences recovered from WWTF10 were predominantly composed of two variant lineages comprising 48 and 49% of the total sequences (FIG. 1C). By May, when cases were dramatically dropping, several of the NYC sewersheds did not contain high enough concentrations of SARS-CoV-2 RNA for analysis, which prevented further determination of city-wide variant distributions from wastewater.

As an external confirmation of our findings, we analyzed raw reads uploaded by Sep. 2021 to NCBI's Sequence Read Archive (SRA) from nearly 5000 other wastewater samples globally spanning 2020-2021, including 172 samples from New York state. Of all samples, only 7, all from NY state sewersheds, had sequences resembling the lineages we described (SRA Accessions: SRR.15202279, SRR15384049, SRR15291304, SRR15128978, SRR15128983, SRR15202284, and SRR15202285).

Are cryptic lineages derived from unsampled COVID-19 infections?

The existence of these cryptic lineages may point to COVID-19 infections of human patients that are not being sampled through standard clinical sequencing efforts. The frequency of weekly confirmed cases in NYC that were sequenced ranged from 2.6% on Jan. 31, 2021 to 12.9% on Jun. 12, 2021 (://github.comlnychealth/coronavirus-data/blob/master/variants/cases-sequenced.csv). Nonetheless, not all cases were diagnosed and not all positive samples were sequenced. Therefore, it cannot be ruled out that the WNY lineages may be derived from patients, who are not being sampled in clinical settings.

Alternatively, these cryptic lineages may be derived from physically distinct locations in the body. That is, perhaps viruses of these lineages predominantly replicate in gut epithelial cells and are not present in the nasopharynx such that standard swabbing techniques can recover sufficient quantities for sequencing. Finally, we speculate that perhaps these mutations are found in minority variants that are unreported in consensus sequences uploaded to EpiCoV (GISAID database) and other databases. Several groups have identified evidence of within host quasispecies in NGS datasets21,22. In one case, as many as 68% of the samples contained evidence of quasispecies in several loci, 76% of which contained nonsynonymous mutations concentrated in the S and orfla genes²¹. To address whether our variants were associated with within-host diversity, we checked for minority variants in the raw reads of sequencing runs performed on samples collected between January 2020 to July 2021 obtained from NY state COVID-19 patients uploaded to the SRA. Of the 7309 samples publicly available as of Jul. 21, 2021, none had sequences that matched the WNY lineages. Some sequences from these SRAs had subsets of mutations associated with the WNY lineages, but never a full suite or at a high frequency.

Arguing against the possibility of unsampled human strains is the geographical stratification of these cryptic lineages. Since January 2021, the lineages have remained geographically constrained over many months in the sewersheds we sampled, which is not consistent with a contagious human pathogen. While there were some COVID-19 related restrictions in NYC (e.g., restaurants operated at 50% capacity), movement was generally not restricted during the study period. Public transportation was operating in a normal capacity. Furthermore, our group regularly processes wastewater samples from over 100 locations and have never seen this kind of geographic constraint of a SARS-CoV-2 lineage that coincides with verified patient sequences. We suspect this lack of dispersal is consistent with infections of non-human animals with restricted movements or home ranges but note that it could also be associated with patients confined to long-term healthcare facilities (e.g., nursing homes, hospices).

### Do Cryptic Lineages Indicate Presence of SARS-CoV-2 Animal Reservoirs?

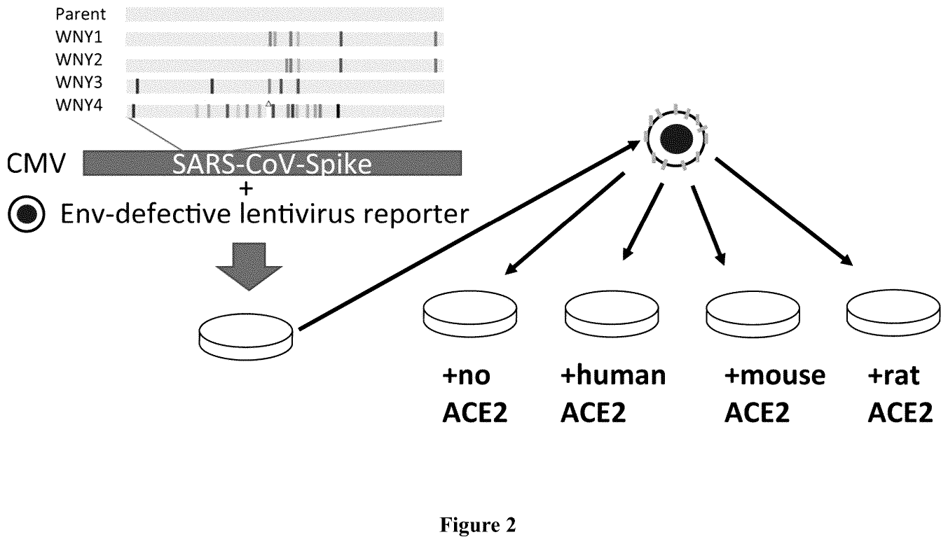

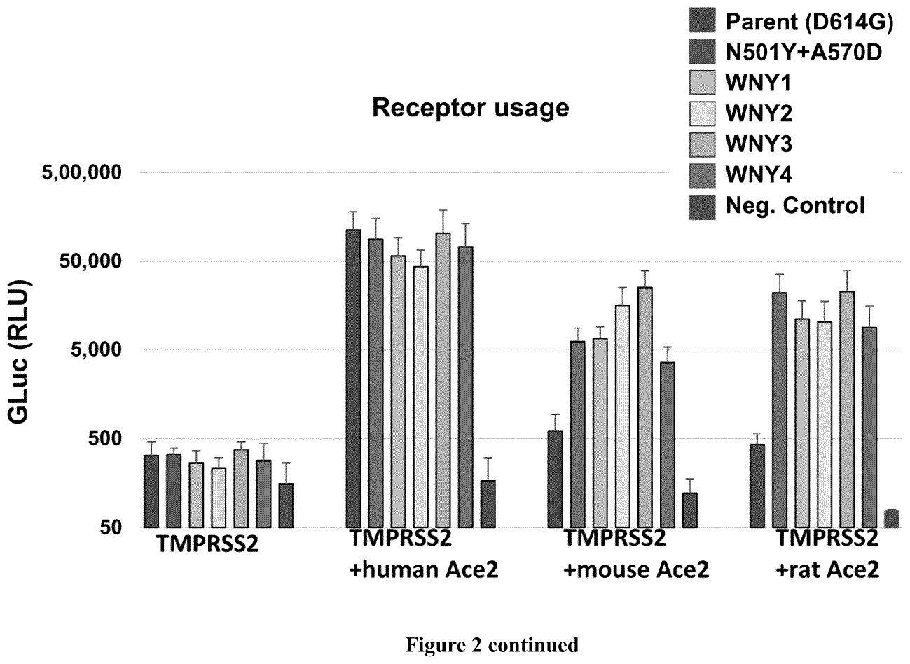

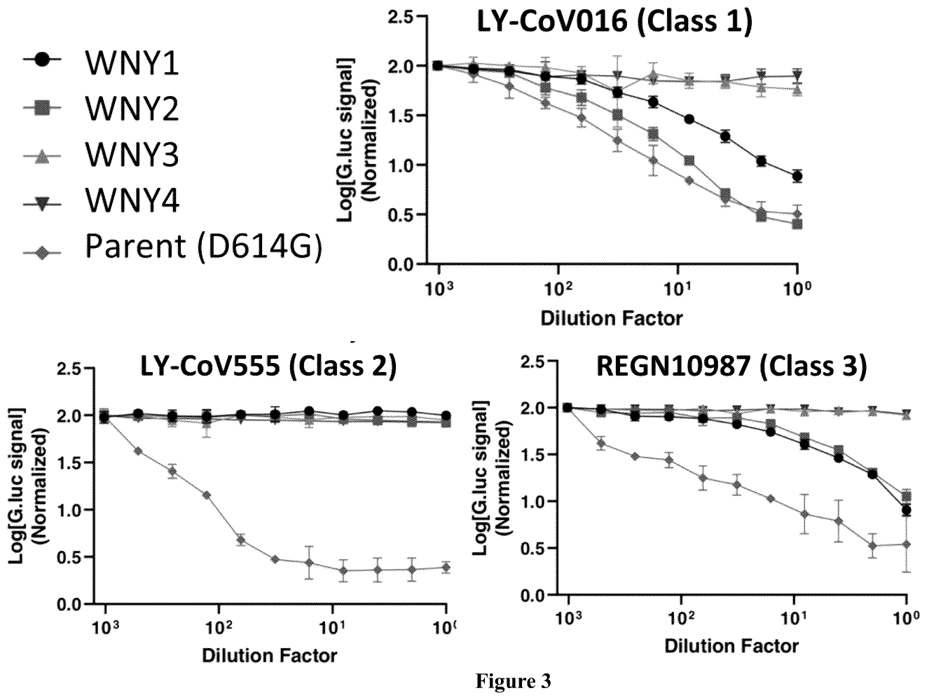

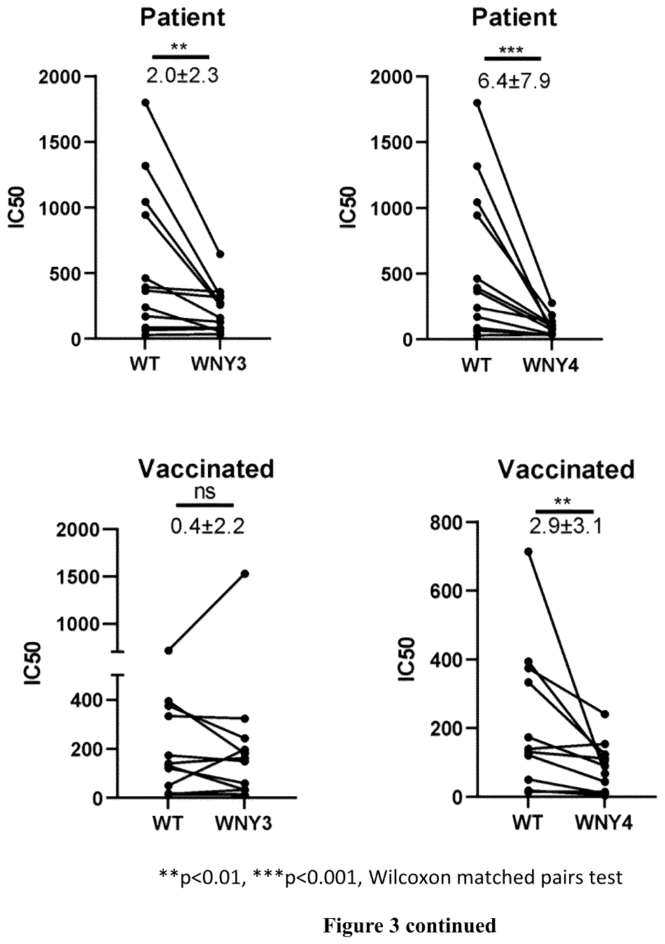

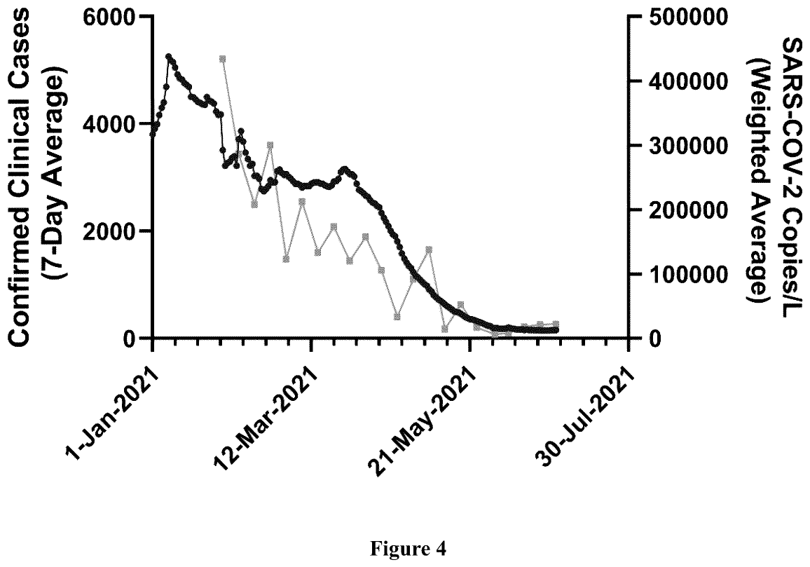

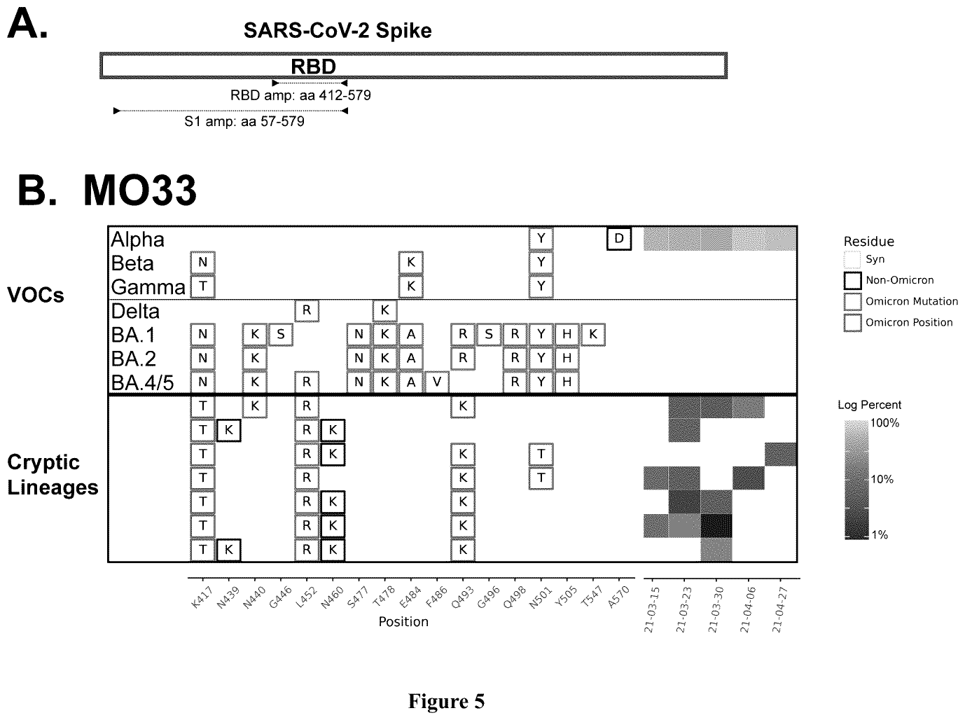

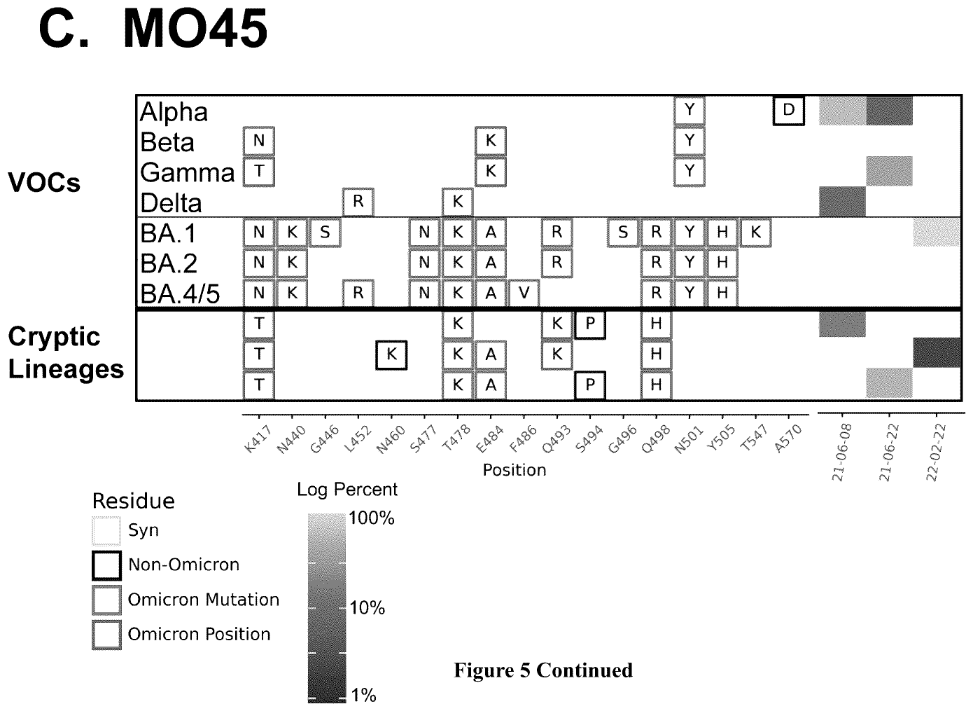

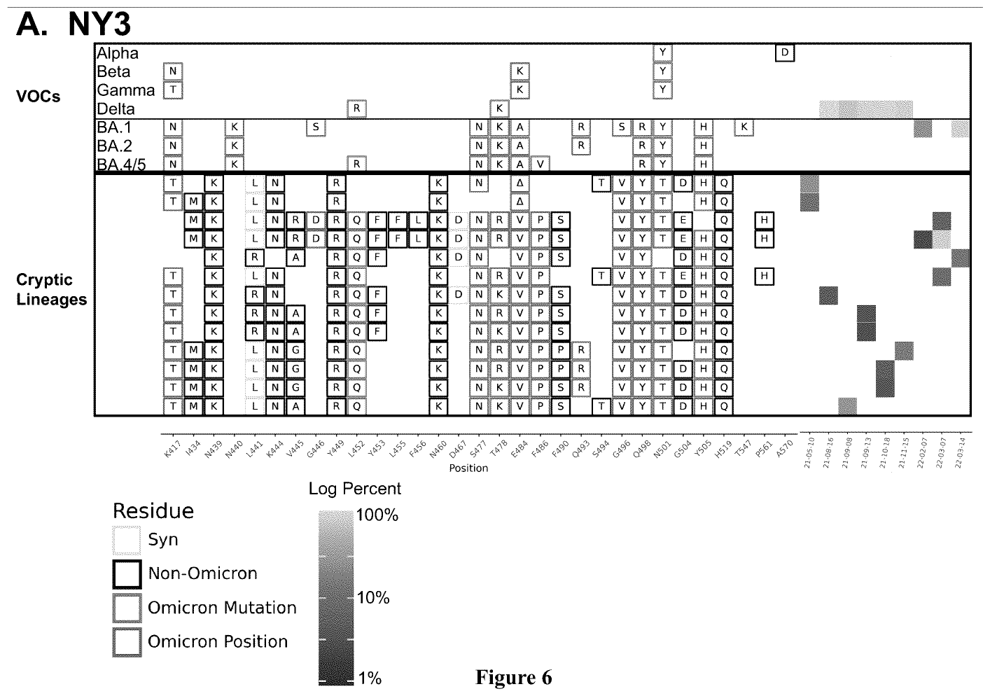

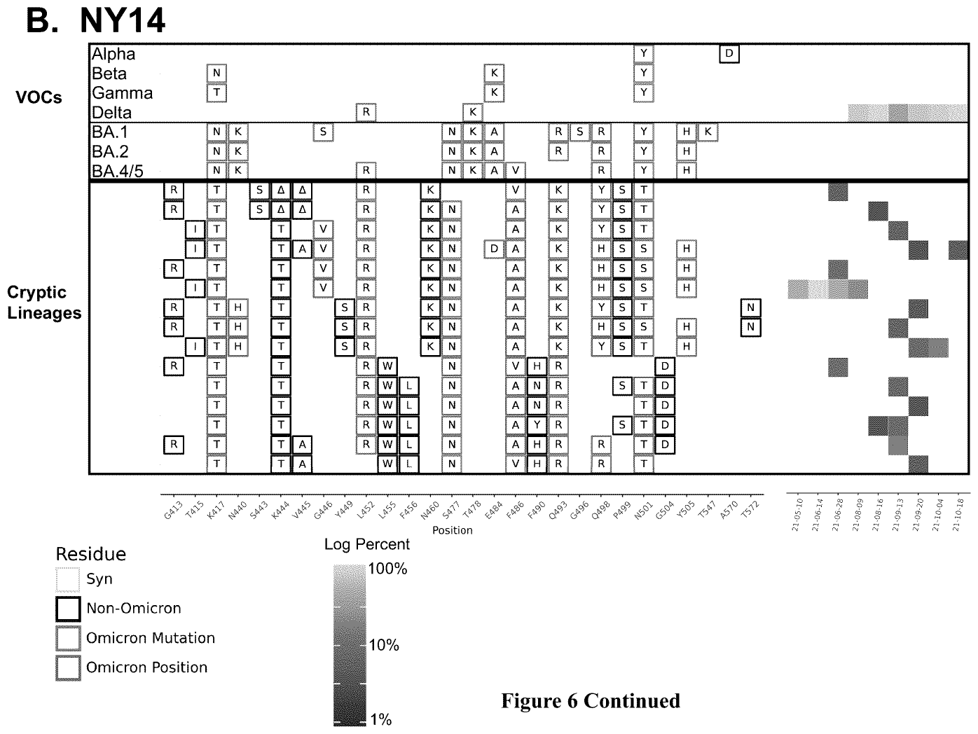

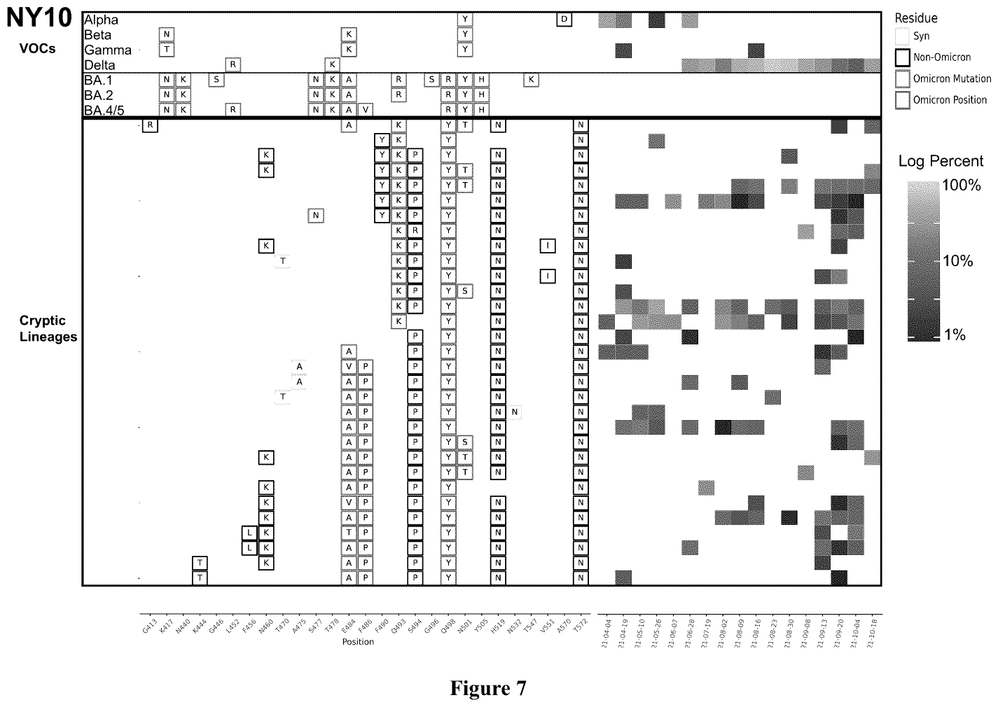

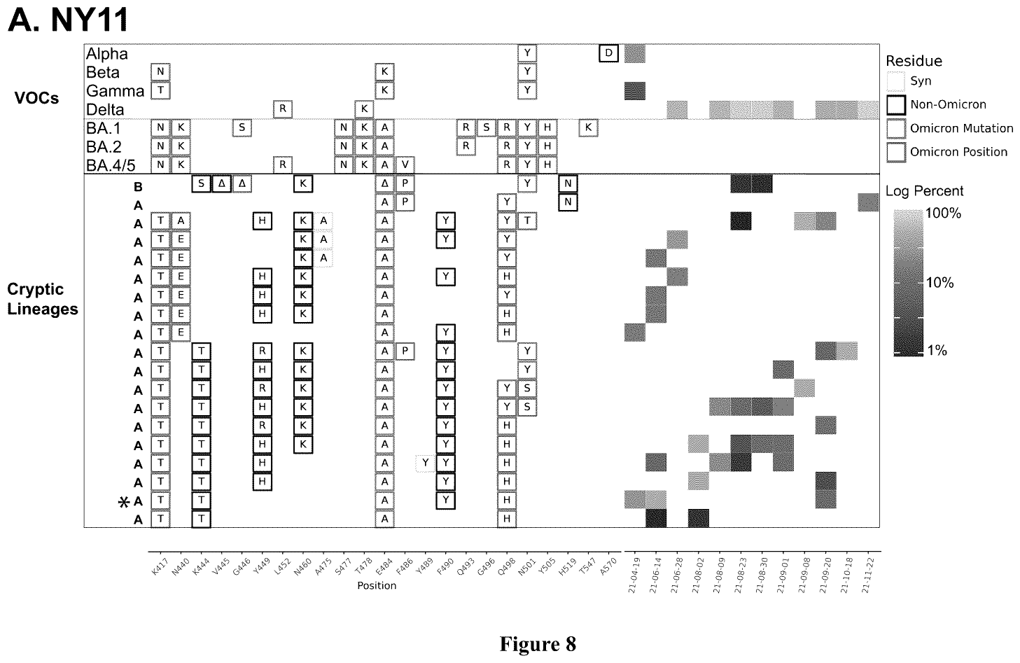

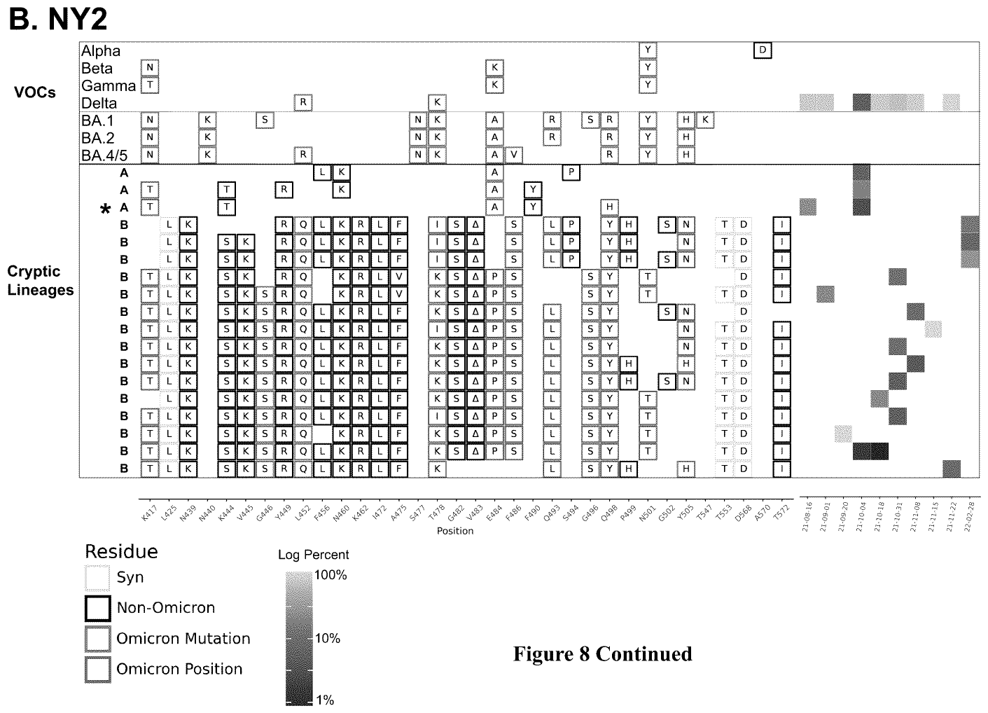

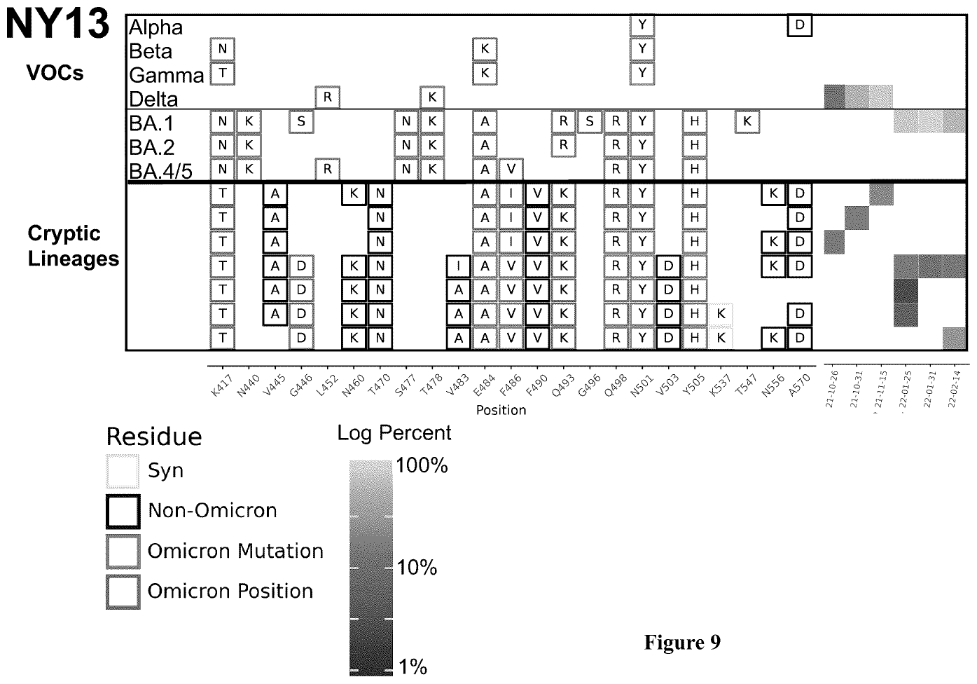

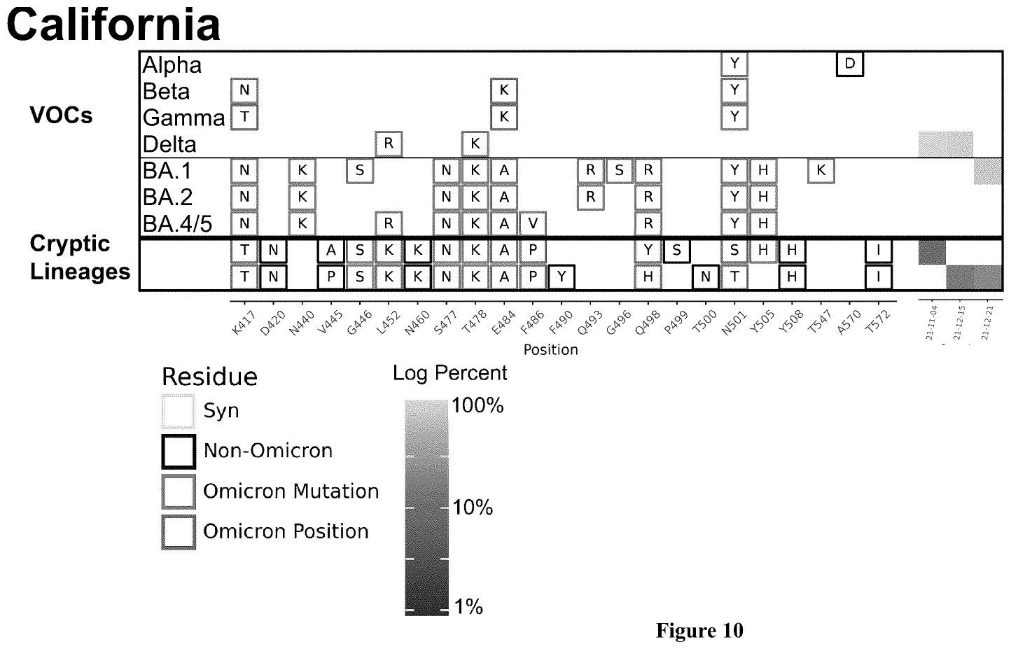

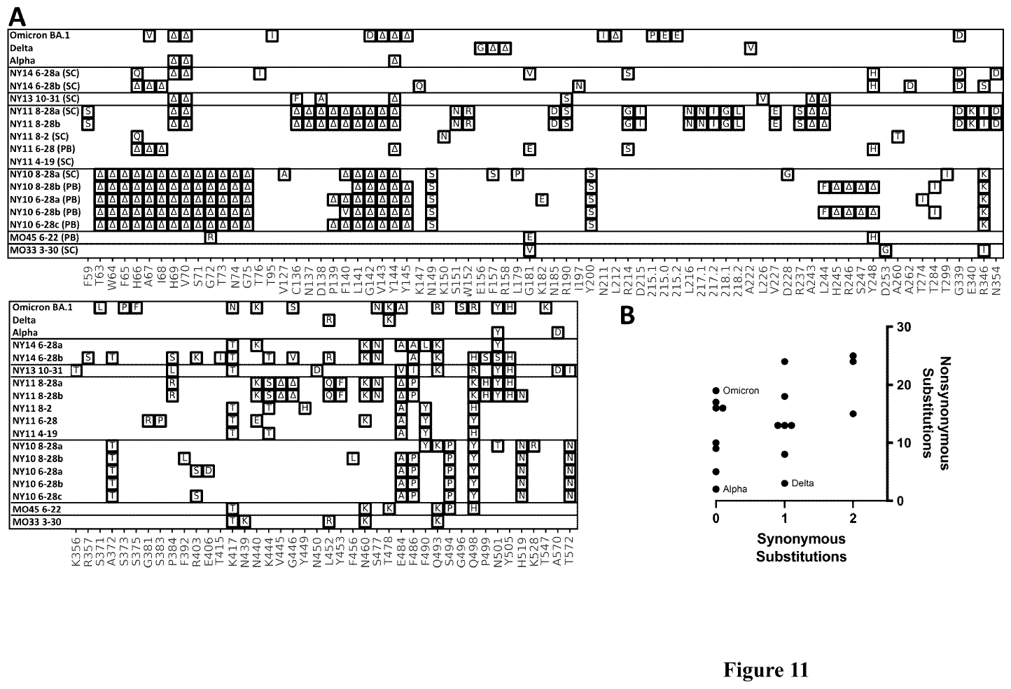

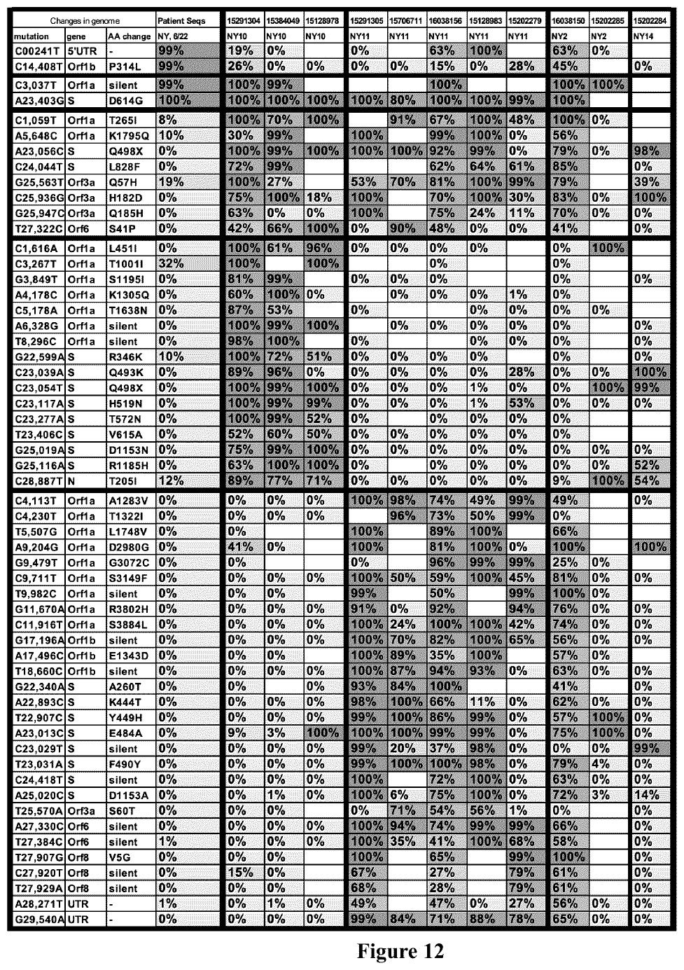

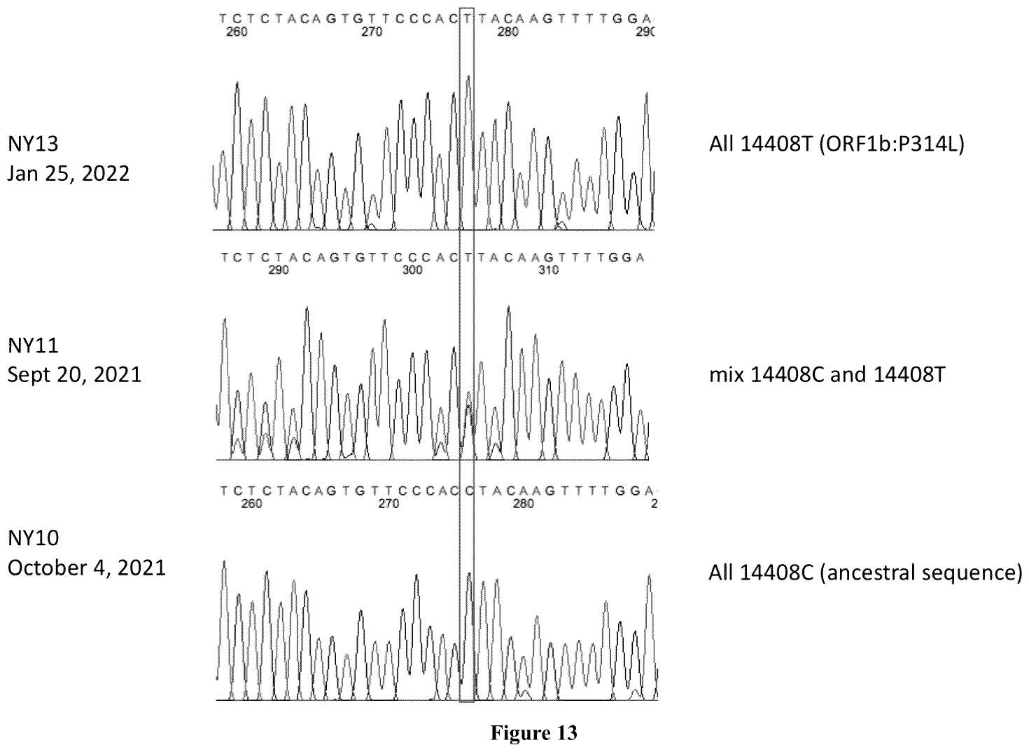

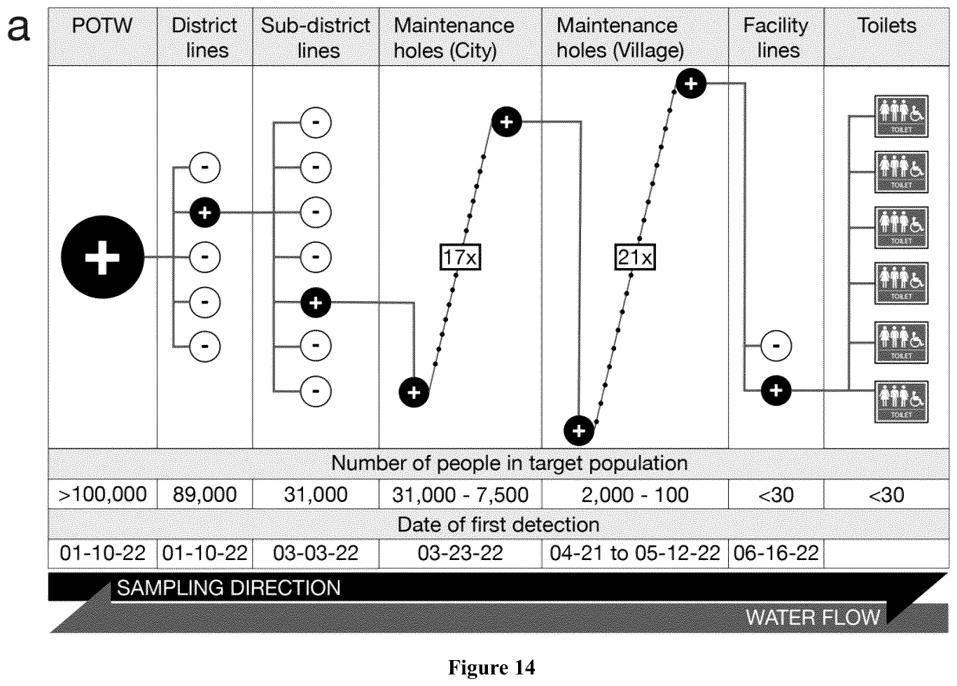

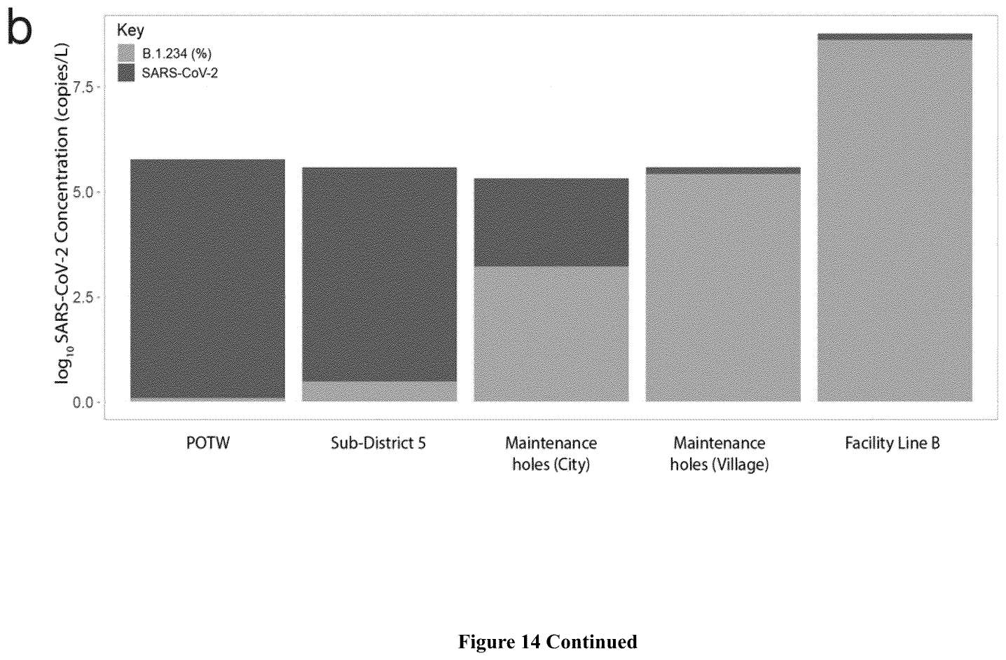

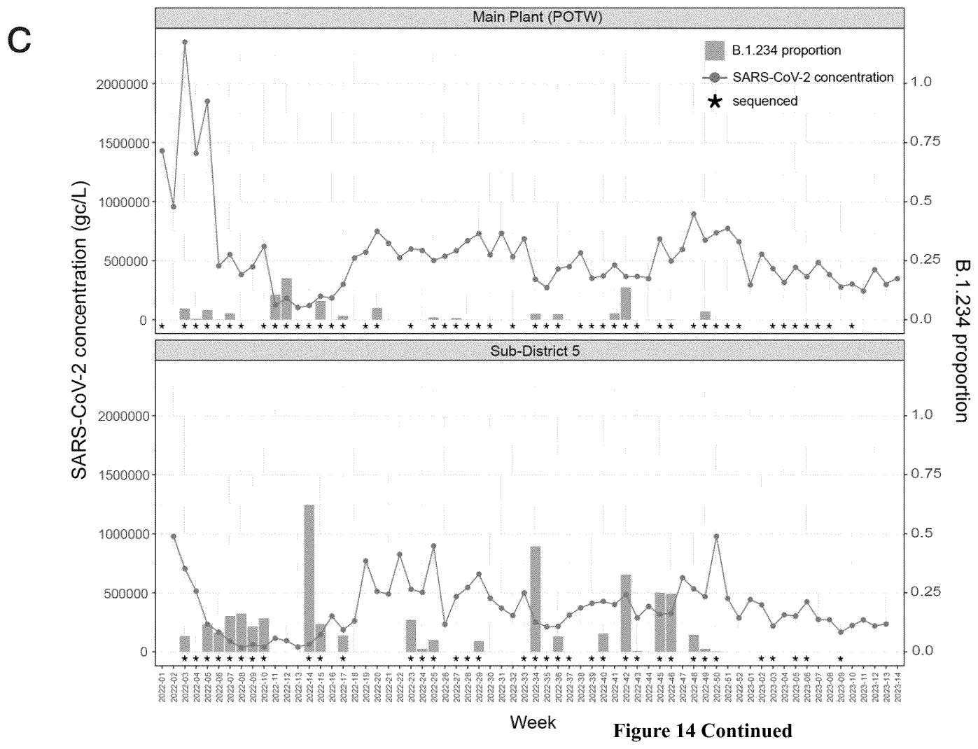

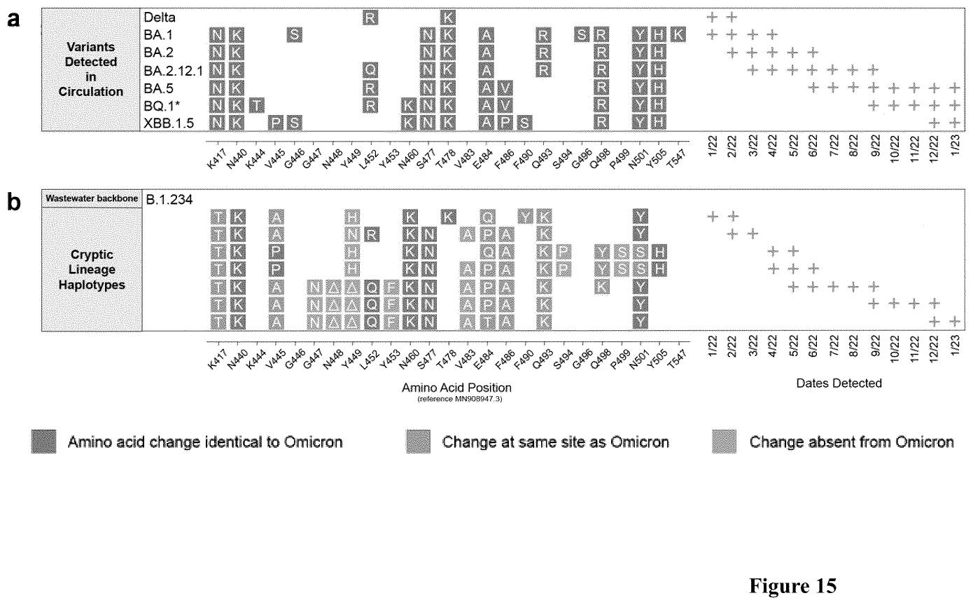

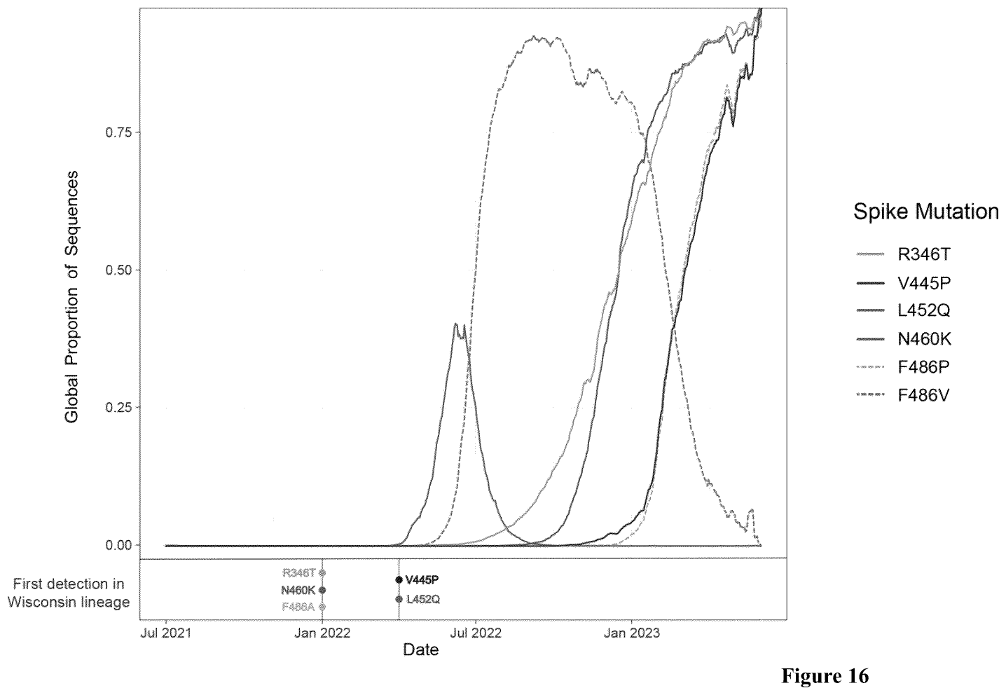

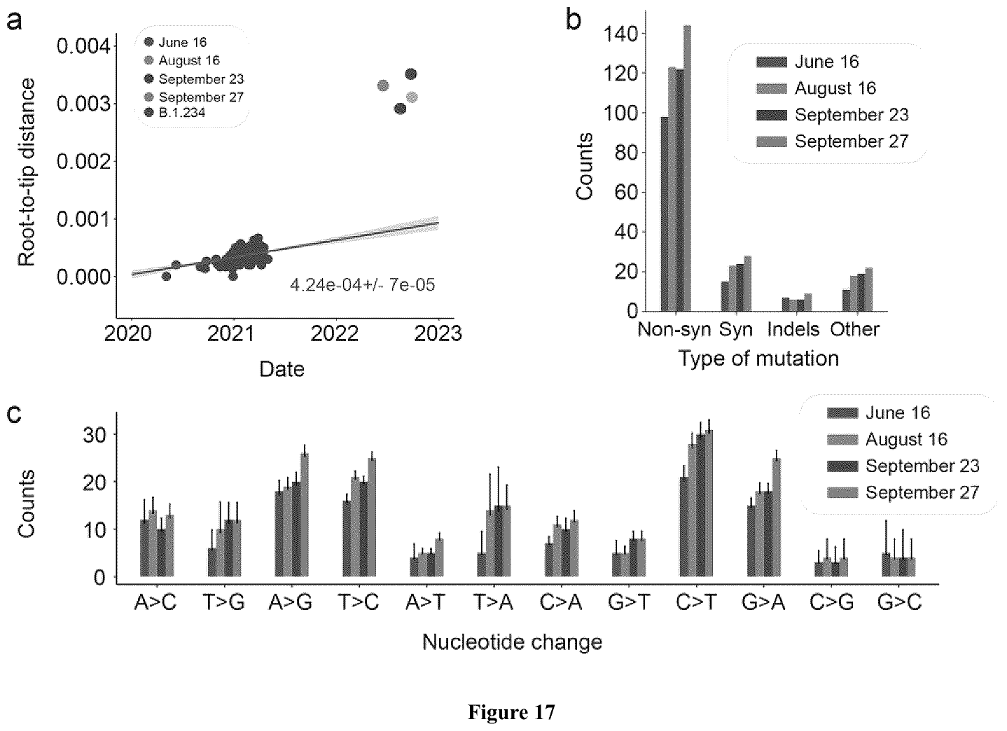

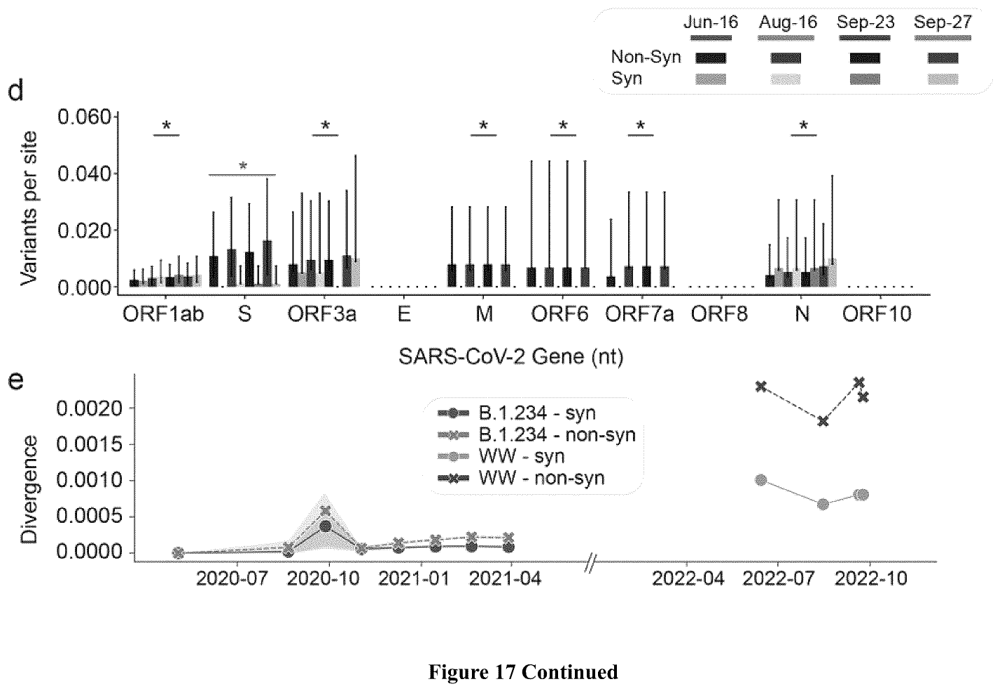

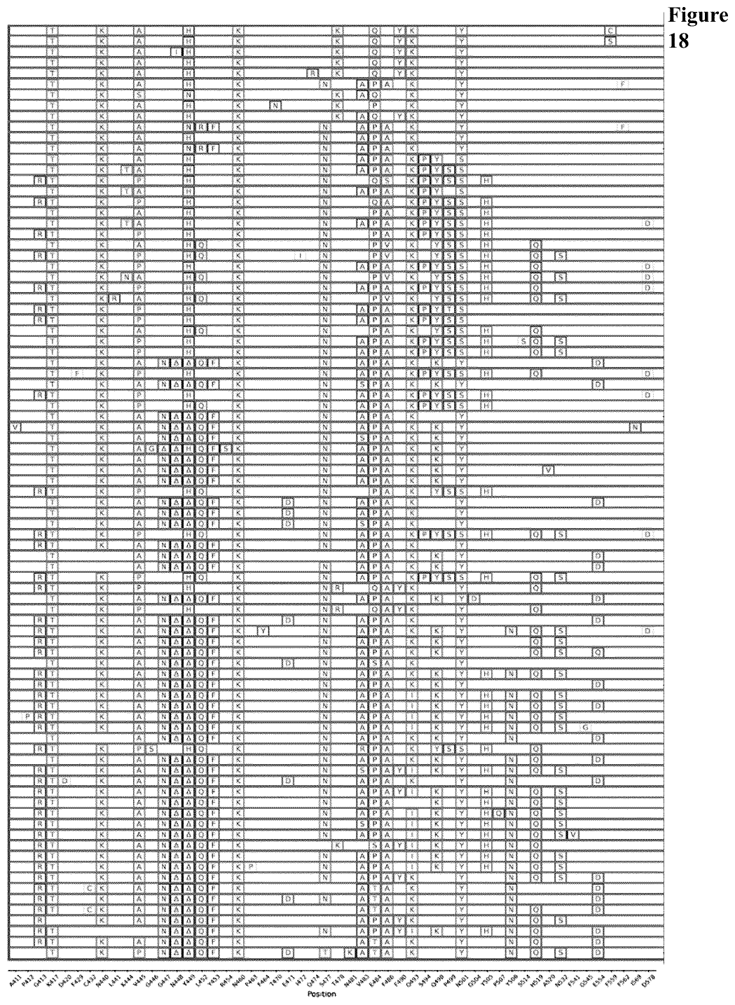

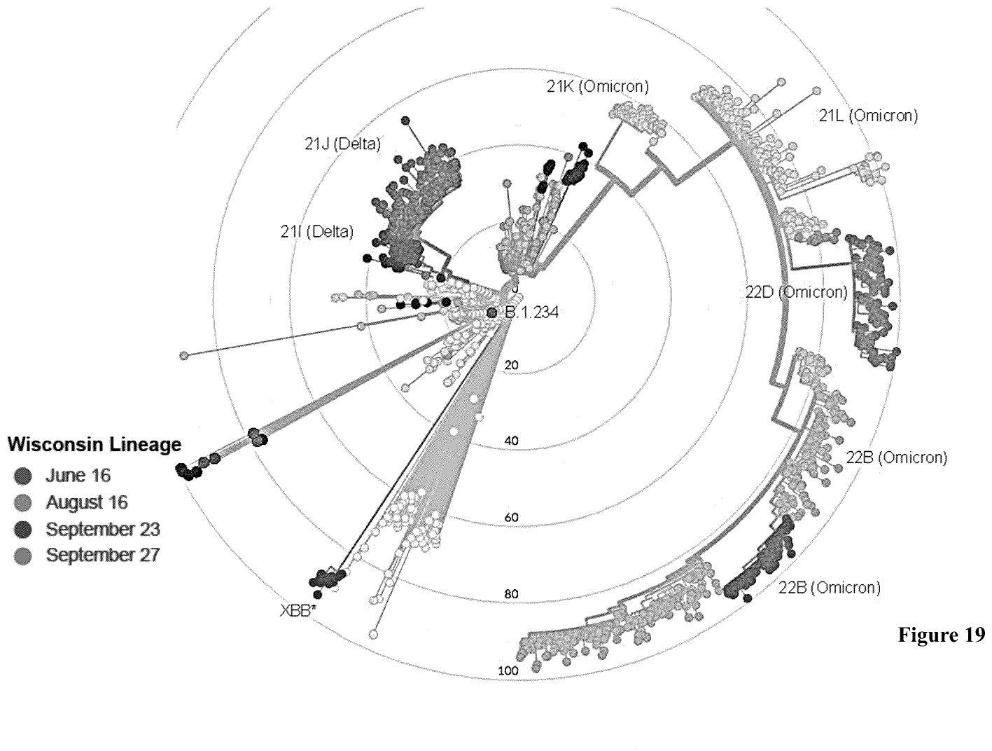

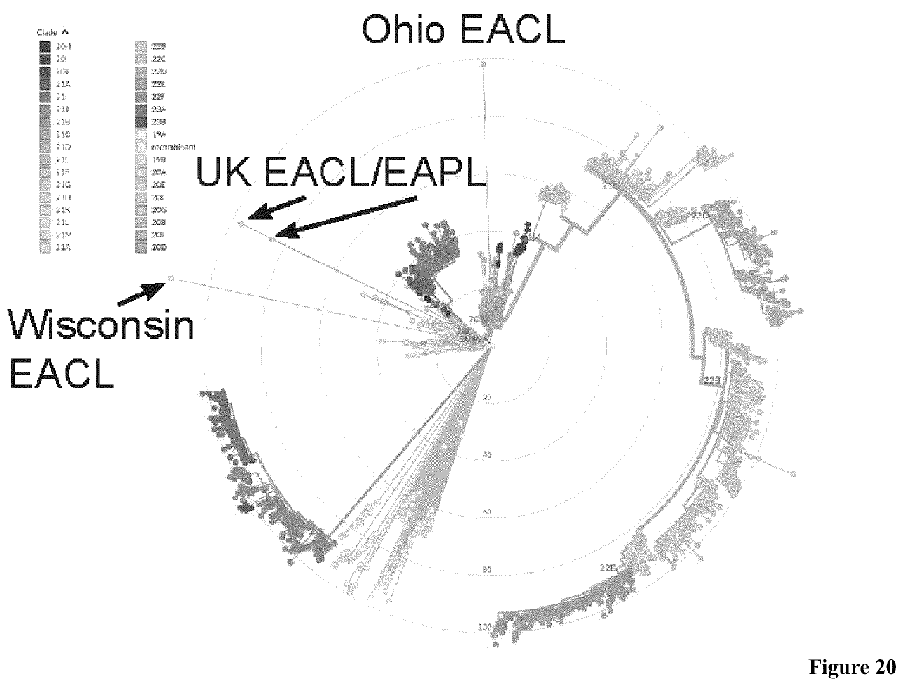

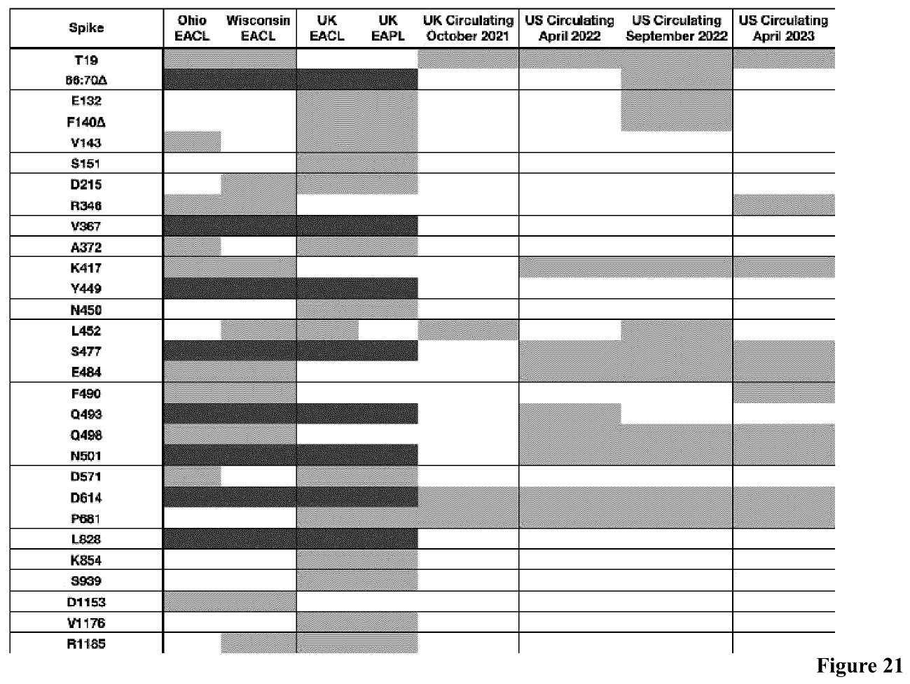

Another hypothesis is that these cryptic lineages are derived from SARS-CoV-2 animal reservoirs. To date, there have been a number of animals infected by SARS-CoV-2, including mink²³, lions and tigers²⁴, and cats and dogs^{25,26}. To gain insight into the possible host range of these lineages, synthetic DNA coding for the amino acid sequences for these four lineages were generated and introduced into a SARS-CoV-2 spike expression construct for functional analysis (FIG. 2). All four of these lineages were found to be fully functional and produced transduction-competent lentiviral pseudoviruses with titers similar to the parent strain (D614G). To determine if these pseudoviruses displayed an expanded receptor tropism, stable cell lines expressing Human, Mouse, or Rat ACE2 were cultured with the pseudoviruses (FIG. 2). While the parent SARS-CoV-2 spike pseudoviruses could only transduce cells with human ACE2, all four of the lineages could efficiently transduce cells with the human, mouse, and rat ACE2. Because some patient-derived SARS-CoV-2 lineages, such as Beta and Gamma, have also gained the ability to infect rodent cells (FIG. 2, N501Y+A570D), this observation cannot be taken as evidence that these lineages were derived from such a host²⁷. Nonetheless, the observation is consistent with the possibility that these lineages are derived from an animal host such as a rodent.

If such reservoirs exist, the animal host would need to meet several criteria. First, the host species would need to be present in the sewershed. Second, the number of susceptible animals present must be high enough to sustain an epidemic for at least six months (i.e., the period for which we observe these sequences). Third, host animals must not disperse beyond the geographical locations where the sequences are found. Finally, there must be a route for shed viruses to enter the sewersheds where the lineages are seen.

We considered several mammalian species known to inhabit NYC that may meet these criteria, including bats (several species), cats (Felis catus), dogs (Canis familiaris), gray squirrels (Sciurus carolinensis), mice (Mus musculus or Peromyscus leucopus), opossums (Didelphis virginiana), rabbits (Sylvilagus floridanus), raccoons (Procyon lotor), rats (Rattus norvegicus), and skunks (Mephitis mephitis). To narrow our search, we reasoned that if viruses are being shed from one of these animals, then we should be able to detect rRNA from the animal in the sewershed as well.

### Mammalian Species Detected in Wastewater

We extracted total RNA from wastewater samples obtained on two different dates from sewersheds where the WNY lineages were observed. This RNA was PCR amplified with 12S rRNA primers (Table 3) and deep sequenced. Sequences mapping to mammalian rRNA were observed in all samples (Table 1). In all cases, the majority of the rRNA sequences mapped to human rRNA. Several species, such as cow, pig, and sheep, were identified that are not indigenous to NYC. These detects are likely derived from food consumption so are ruled out as possible hosts. After non-indigenous mammals were removed, four remaining mammalian species were repeatedly detected: humans, cats, dogs, and rats (Table 1).

TABLE 3
Primers and probes used in this study

| | Forward | | | | |
|---|---|---|---|---|---|
| | Primer | SEQ ID | Reverse | SEQ ID | |
| Name and Site | (Probe) | NO: | Primer | NO: | Source |
| 2019- | GACCCCAA | 1 | TCTGGTTA | 2 | CDC 2019- |
| nCoV_N1 | AATCAGCG | | CTGCCAGT | | nCoV |
| (SARS-CoV-2 | AAAT | | TGAATCTG | | Real-Time |
| spike) | | | | | RT-PCR |
| | | | | | Diagnostic |
| | | | | | Panel |
| 2019- | FAM- | 3 | | | CDC 2019- |
| nCoV_N1 | ACCCCG | | | | nCoV |
| Probe (SARS- | CAT/ZEN/TA | | | | Real-Time |
| CoV-2 spike) | CGTTTGGT | | | | RT-PCR |
| | GGACC- | | | | Diagnostic |
| | 3IABKFQ | | | | Panel |
| iSeq 100 RBD | tcgtcggcagcgt | 4 | gtctcgtgggctcg | 5 | This study |
| sequencing | cagatgtgtataag | | gagatgtgtataag | | |
| primers | agacagCCAG | | agacagGAAA | | |
| (SARS-CoV-2 | ATGATTTT | | GTACTACT | | |
| spike receptor | ACAGGCTG | | ACTCTGTA | | |
| binding | CG | | TGGTTGG | | |
| domain)* | | | | | |
| MiSeq RBD | CTGCTTTAC | 6 | TCCTGATA | 7 | Reference |
| primary PCR | TAATGTCT | | AAGAACAG | | 8 |
| primers | ATGCAGAT | | CAACCT | | |
| (SARS-CoV-2 | TC | | | | |
| spike receptor | | | | | |
| binding | | | | | |
| domain) | | | | | |
| MiSeq RBD | acactctttccctac | 8 | gtgactggagttca | 9 | Reference |
| Nested PCR | acgacgctcttccg | | gacgtgtgctcttc | | 8 |
| primers | atctGTGATG | | cgatctATGTC | | |
| (SARS-CoV-2 | AAGTCAGA | | AAGAATCT | | |
| spike receptor | CAAATCGC | | CAAGTGTC | | |
| binding | | | TG | | |
| domain)* | | | | | |
| 12S-V5- | TCGTCGGC | 10 | GTCTCGTG | 11 | Reference |
| Tailed-F1 and | AGCGTCAG | | GGCTCGGA | | 43 |
| R1 | ATGTGTAT | | GATGTGTA | | |
| | AAGAGACA | | TAAGAGAC | | |
| | GACTGGGA | | AGAGAACA | | |
| | TTAGATAC | | GGCTCCTC | | |
| | CCC | | TAG | | |
| MiSeq 12s | acactctttccctac | 12 | gtgactggagttca | 13 | Reference |
| PCR primers* | acgacgctcttccg | | gacgtgtgctcttc | | 43 |
| | atctACTGGG | | cgatctTAGAA | | |
| | ATTAGATA | | CAGGCTCC | | |
| | CCCC | | TCTAG | | |

TABLE 1
Predominant species detected in NYC wastewater
via deep sequencing if 12S amplicons

| | Common | WWTP 3 | WWTP 10 | WWTP 11 |
|---|---|---|---|---|
| Genus | name | 6-7/6-28 | 6-7/6-28 | 6-7/6-28 |
| Homo | Human | M/M | M/M | M/M |
| Bos | Cow | ++/+++ | ++/++ | ++/++ |
| Sus | Pig | +++/+++ | +++/+++ | +++/+++ |
| Rattus | Rat | +++/+++ | +/− | ++/++ |
| Canis | Dog | ++/++ | +++/+++ | ++/+ |
| Felis | Cat | ++/++ | ++/+ | +/− |
| Ovis | Sheep | +/− | +/− | +/− |

Results from samples obtained on June 7 and June 28 are shown. The fraction of the total sequences detected are denoted as follows: Majority >50% (M), >1% (+++), >0.1% (++), <0.1% (+), not detected (−).

Cats and dogs are susceptible to SARS-CoV-22829, and cats are able to transmit to other animals²⁶. Many rodents are not permissive for infection by the canonical SARS-CoV-2 strain^{20,30}, but some variants have an expanded tropism that includes mice²⁷. A 2013 census estimated that there are 576,000 pet cats in NYC households³¹, but this estimate does not include stray cats. Extrapolating from a limited study conducted in 2017 implies a stray cat population of about 2500 animals³², but this number does not accord with the approximately 18,000 animals received annually by NYC Animal Care Centers³¹. There are currently 345,727 active dog licenses in NYC33, but this figure is likely a significant underestimate and the true number may be at least double this figure. Despite these uncertainties, both cat and dog populations are dwarfed by the NYC rat population, which is estimated to number between 2-8 million animals³⁴.

WWTP 10 wastewater contained cat, rat, and dog rRNA, but rat rRNA reads were less than 0.1% of total reads and were only detected on one of the two dates tested (Table 1). This low detection was expected because the WWTP 10 sewershed is not a combined system (i.e., stormwater generally does not mix with wastewater). Moreover, the sewershed serves a suburban residential area and is believed to have one of the lowest rat densities in the city based on the volume of rat complaints received by city services (https://data.cityofnewyork.us/Social-Services/Rats-Heat-Map/g642-4e55). WWTP 3 and 11 wastewater also contained cat, rat, and dog rRNA, though the composition varied. In WWTP 3 wastewater, rat rRNA was the most prevalent after humans, representing over 1% of the total rRNA reads (Table 1). In WWTP 11, rat and dog rRNA were both above 0.1% of reads, but cat rRNA reads were less than 0.1% of total reads and were only detected on one of two dates tested (Table 1). All of these numbers are eclipsed by the overwhelming prevalence of human rRNA in the same samples. As no animal rRNAs are highly prevalent in all three sewersheds, it is difficult to reconcile a single animal being the reservoir for all cryptic lineages in NYC wastewater.

Cryptic Lineages Detected from Wastewater are Resistant to Some Neutralizing Antibodies

In addition to polymorphisms from the cryptic lineages that are known to affect viral tropism, many of the polymorphisms are also known to affect antibody evasion. In particular, the WNY polymorphisms at positions K417, N439, N440, K444, L452, N460, E484, Q493, S494, and N501 have all been reported to evade neutralization by particular antibodies^{9,11,12,13,14}. Most neutralizing antibodies against SARS-CoV-2 target the RBD of the spike protein, and most of these neutralizing antibodies are divided into three classes based on binding characteristics³⁵.

To test if the cryptic lineages have gained resistance to neutralizing antibodies, we obtained three clinically approved neutralizing monoclonal antibodies representing these 3 classes, LY-CoV016 (etesevimab, Class 1)36, LY-CoV555 (bamlanivimab, Class 2)37, and REGN10987 (imdevimab, Class 3)38, and tested their ability to neutralize the cryptic lineages. All four of the lineages displayed complete resistance to LY-CoV555, despite the parent lineage remaining potently sensitive to this antibody (FIG. 3). WNY1 and WNY2 remained at least partially sensitive to LY-CoV016 and REGN10987, but WNY3 and WNY4 appeared to be completely resistant to all three neutralizing antibodies (FIG. 3).

Finally, we tested the ability of plasma from fully vaccinated individuals (Pfizer) or patients previously infected with SARS-CoV-2 to neutralize WNY3 and WNY4. All patients' plasma retained some capacity to neutralize these pseudoviruses (FIG. 3). However, previously infected patients had an average 2-fold and 6.4-fold reduction in ID50 (WT vs. variant) with WNY3 and WNY4, respectively. Vaccinated patient plasma did not have a statically significant reduction with WNY3 but had an average 2.9-fold reduction with WNY4. It must be noted that neutralizing antibody activity from vaccinated individuals is not solely directed against the spike RBD. Therefore, if the full spike proteins from these cryptic lineages with the additional mutations they carry were tested, the neutralization capacity could be enhanced or further diminished. Thus, the characteristics of these lineages provide them the capacity to be a potential increased threat to human health.

### Challenges

While we believe that our data, analysis, and interpretation of our findings warrant sharing with the scientific community, we recognize that our study has several limitations. The source of the novel lineages has not been identified. Investigations are ongoing to test possible animal reservoirs from these sewershed and to better pinpoint the geographical source of the cryptic variants by sequencing RNA from wastewater obtained upstream from our WWTP's of interest.

It is also recognized that the targeted sequencing approach does not identify mutations outside of the targeted region. In some cases, whole genome sequencing of wastewater has been employed, but the results have been ambiguous. Typical whole genome sequencing relies on amplification and subsequent computational assembly of genomes from overlapping 150-300 bp reads. When an infected individual's sample is sequenced, mutations appearing in different reads are assumed to be linked given that the reads likely come from a single virus genotype. By contrast, wastewater generally contains virions shed from numerous infected individuals, mutations identified cannot be reliably assigned to a specific genome³⁹. To date it has not been possible to isolate viable virus from wastewater such that single virus genotypes can be sequenced⁴⁰. Therefore, we cannot link mutations unless they are found on the same amplicon.

A further challenge is that the depth of coverage across the SARS-CoV-2 genomes sequenced from wastewater tends to be uneven. As such, phylogenetically and clinically important regions of the genome may fail to be adequately sequenced at appropriate levels of coverage. We chose to focus on a region of the spike RBD because of the prevalence of mutations that are phylogenetically and clinically important. We can reliably sequence this amplicon with high coverage.

To address the limitations presented by targeting just a small region of the SARS-CoV-2 spike, we are incorporating targeted sequencing of other variable regions of interest in the genome, particularly those regions that contain mutations unique to specific variants of concern. In addition, we are PM amplifying, cloning, and sequencing a 1.5 kb region of the spike protein gene to confirm the linkage of mutations of interest.

### SUMMARY

To date, most data on SARS-CoV-2 genetic diversity has come from the sequencing of clinical samples, but such studies may suffer limitations due to biases, costs, and throughput. Here, we demonstrate the circulation of several cryptic lineages of SARS-CoV-2 in the NYC metropolitan area that have not been detected by standard clinical surveillance. While the origins of these cryptic lineages have not been determined, we have demonstrated that they have expanded receptor tropism which is consistent with expansion to an animal reservoir. Other SARS-CoV-2 animal reservoirs have been identified^{23,41}. However, no single animal was strongly represented in all our rRNA sequencing analysis, which raises doubts that a single animal reservoir is the source of all the cryptic lineages.

Finally, we demonstrated that these cryptic lineages have gained significant resistance to some patient-derived neutralizing monoclonal antibodies. We note especially the high number of shared loci mutated in both our WNY lineages and the Omicron VOC. it's possible that these shared mutated loci are a product of convergent evolution to the shared selective pressure of antibody-mediated neutralization. Thus, these cryptic lineages could be relevant to public health and necessitate further study.

### Methods

### Ethics Statement

All procedures performed in studies involving human participants, including blood collection and processing, were approved by The Institutional Review Board of the University of Missouri (protocols #2043082 and 230262). Written consent was received from all human subjects prior to being enrolled in the study. The cohort of participants were selected based on equivalent levels of antibodies to SARS-CoV-2 RBD, age, or gender did not contribute to differences between samples (Table 2). COVID+ participants were collected prior to Dec. 11, 2020 and did not receive a COVID vaccine. Vaccinated individuals were vaccinated with Pfizer and have not had a previous PCR+ COVID test. Patients were compensated $10/draw.

TABLE 2
Demographic information for participants
in antibody neutralizing study.

| | | COVID-19 patients | Vaccinated |
|---|---|---|---|
| Total | Number | 12 | 12 |
| Age | Average (Years) | 39 | 51 |
| | Range (Years) | 19-64 | 24-65 |
| Gender | Male | 1 | 3 |
| | Female | 11 | 9 |
| Vaccine type | | — | Pfizer |

### Wastewater Sample Processing and RNA Extraction

Wastewater (24 h composite samples) was collected from the inflow at 14 NYC wastewater treatment plants and RNA isolated according to our previously published protocol2. While samples have been obtained and processed on a weekly basis since June 2020, we report herein the outcome of sequencing runs performed approximately every 2 weeks between January and June 2021. The specific dates of sampling were January 31st, February 28th, March 14th, April 5th, April 19th, May 10th, May 26th, June 7th, June 14th, and June 28th.

Briefly, 250 mL from 24 h composite raw sewage samples obtained from NYC WWTPs were centrifuged at 5000×g for 10 min at 4° C. to pellet solids. Forty milliliter of supernatant was passed through a 0.22 μM filter (Millipore, SLGPR33R). Filtrate was stored at 4° C. for 24 h after adding 0.9 g sodium chloride (Fisher Scientific, BP358-10) and 4.0 g PEG 8000 (Fisher Scientific, BP233-1) then centrifuged at 12,000×g for 120 minutes at 4° C. to pellet the precipitate. The pellet was resuspended in 1.5 mL TRIzol (Fisher Scientific,15596026), and RNA was purified according to the manufacturer's instructions.

### Targeted PCR: iSeq Sequencing

RNA isolated from wastewater was used to generate cDNA using ProtoScript® II Reverse Transcriptase kit (New England Biolabs, M0368S). The RNA was incubated with an RBD specific primer (ccagatgattttacaggctgcg, Genewiz SEQ ID NO:4) and dNTPs (0.5 mM final concentration, included in the kit) at 65° C. for 5 min and placed on ice. The RT buffer, DTT (0.01 M final concentration, included in the kit), and the RT were added to the same tube and incubated at 42° C. for 2 h followed by 20 min at 65° C. to inactivate the enzyme. The RBD region was amplified using Q5® High-Fidelity DNA Polymerase (New England Biolabs, M0491S) using primers that incorporate Illumina adapters (Table 4). PCR performed as follows: 98° C. (0:30)+40 cycles of [98° C. (0:05)+53° C. (0:15)+65° C. (1:00)]×40 cycles+65° C. (1:00).

TABLE 4
Viral N1 copies/L by date for WWTPs associated
with unknown lineages described in FIG. 1C

| Date | WWTP 3 | WWTP 10 | WWTP 11 |
|---|---|---|---|
| Jan. 31, 2021 | 1,496,155.0 | N/A | IN/A |
| Feb. 28, 2021 | 464,927.1 | 40,046.3 | 240,540.3 |
| Mar. 14, 2021 | 302,431.6 | 81,635.5 | 158,205.5 |
| Apr. 5, 2021 | 149,385.9 | 121,498.9 | 280,317.7 |
| Apr. 19, 2021 | 61,092.8 | 72,645.4 | 49,146.5 |
| May 10, 2021 | 8,183.5 | 8,085.3 | 117,408.2 |
| May 26, 2021 | 12,978.3 | 28,591.6 | 130,956.9 |
| Jun. 7, 2021 | 8,812.9 | 18,082.1 | 19,436.2 |
| Jun. 14, 2021 | 17,366.4 | 165,067.6 | 4,273.3 |
| Jun. 28, 2021 | 12,639.4 | 47,753.7 | 101,757.1 |

The RBD amplicons were purified using AMPure XP beads (Beckman Coulter, A63881). Index PCR was performed using the Nextera DNA CD Indexes kit (Illumina, 20018707) with 2×KAPA HiFi HotStart ReadyMix (Roche, KK2601), and indexed PCR products purified using AMPure beads (Beckman Coulter, A63881). The indexed libraries were quantified using the Qubit 3.0 and Qubit dsDNA HS Assay Kit (Invitrogen, Q32854) and diluted in 10 mM Tris-HCl to a final concentration of approximately 0.3 ng/μL (1 nM). The libraries were pooled together and diluted to a final concentration of 50 pM. Before sequencing on an Illumina iSeq100, a 10% spike-in of 50 pM PhiX control v3 (Illumina, FC-110-3001) was added to the pooled library. The Illumina iSeq instrument was used to generate paired-end 150 base pair length reads.

### Targeted PCR: MiSeq Sequencing

The primary RBD RT-PCR was performed using the Superscript IV One-Step RT-PCR System (Thermo Fisher Scientific, 12594100). Primary RT-PCR amplification was performed as follows: 25° C. (2:00)+50° C. (20:00)+95° C. (2:00)+[95° C. (0:15)+55° C. (0:30)+72° C. (1:00)]×25 cycles using the MiSeq primary PCR primers (Table 3). Secondary PCR (25 μL) was performed on RBD amplifications using 5 μL of the primary PCR as template with MiSeq nested gene specific primers containing 5′ adapter sequences (Table 3) (0.5 μM each), dNTPs (100 μM each) (New England Biolabs, N0447L) and Q5 DNA polymerase (New England Biolabs, M0541S). Secondary PCR amplification was performed as follows: 95° C. (2:00)+[95° C. (0:15)+55° C. (0:30)+72° C. (1:00)]×20 cycles. A tertiary PCR (50 μL) was performed to add adapter sequences required for Illumina cluster generation with forward and reverse primers (0.2 μmM each), dNTPs (200 μmM each) (New England Biolabs, N0447L) and Phusion High-Fidelity DNA Polymerase (1U) (New England Biolabs, M0530L). PCR amplification was performed as follows: 98° C. (3:00)+[98° C. (0:15)+50° C. (0:30)+72° C. (0:30)]×7 cycles+72° C. (7:00). Amplified product (10 μl) from each PCR reaction is combined and thoroughly mixed to make a single pool. Pooled amplicons were purified by addition of Axygen AxyPrep MagPCR Clean-up beads (Axygen, MAG-PCR-CL-50) in a 1.0 ratio to purify final amplicons. The final amplicon library pool was evaluated using the Agilent Fragment Analyzer automated electrophoresis system, quantified using the Qubit HS dsDNA assay (Invitrogen), and diluted according to Illumina's standard protocol. The Illumina MiSeq instrument was used to generate paired-end 300 base pair length reads. Adapter sequences were trimmed from output sequences using cutadapt.

Wastewater rRNA sequencing cDNA from wastewater was also used to generate libraries using the primers indicated in Table 3. rRNA Libraries were amplified using ProtoScript® II Reverse Transcriptase (New England Biolabs, M0368S) and pooled and sequenced on the iSeq100 as described above.

### Bioinformatics

iSeq reads were uploaded to the BaseSpace Sequence Hub and demultiplexed using a FASTQ generation script. Reads were processed using the published Geneious workflows for preprocessing of NGS reads and assembly of SARS-CoV-2 amplicons⁴². Paired reads were trimmed, and the adapter sequences removed with the BBDuk plugin. Trimmed reads were aligned to the SARS-CoV-2 reference genome MN908947. Variants present at frequencies of 1% or above were called using the Annotate and Predict Find Variations/SNPs in Geneious and verified by using the V-PIPE SARS-CoV-2 application⁴³.

Reads from iSeq and Mi Seq sequencing were processed as previously described⁸. Briefly, VSEARCH tools were used to merge paired reads and dereplicate sequences⁴⁴. Dereplicated sequences from RBD amplicons were respectively mapped to the reference sequence of SARS-CoV-2 (NC 045512.2) spike ORF using Minimap245. Mapped RBD amplicon sequences were then processed with SAM Refiner using the same spike sequence as a reference and the command line parameters “--alpha 1.8 --foldab 0.6”. The output from SAM Refiner (available at https://github.com/degregory/Cryptic_WNY_sup/tree/main/SAM_Refiner_outputs) were reviewed to determine the known and novel lineage makeup of the sampled sewersheds. To verify and visualize the variant alleles, FreeBayes46 was used to call variants on the mapped reads and Integrative Genomics Viewer47 was used to generate genomic plots.

For sequencing from rRNA templates, dereplicated reads with a minimum unique count of 10 were mapped with Bowtie248 to a collected reference index of mitochondrial and rRNA related animal sequences from NCBI' s nucleotide and refseq databases. Mapped rRNA sequences were reviewed for matching of specific organisms. Sequences with poor mapping to sequences in the index and a random selection of sequences with good mapping were checked by Blast to verify the organism match. Matches were corrected based on the blast results as needed.

For both iSeq and MiSeq datasets, we examined Outbreak.info for the prevalence of each mutation and their associated lineages in New York, the United States and worldwide.

For sequences from GISAID, fasta formatted sequences from NYC patients were obtained from the GISAID database for submissions between January to April 2021. These sequences were processed similarly to the dereplicated sequences above. Minimap2 was used to map the sequences to the spike ORF, then SAM Refiner was used to process the mapped sequences using “-- min_count 1 --min_samp_abund 0” parameters to include all variations in the output.

Fastq formatted sequences were obtained for all sequenced SARS-CoV-2 clinical samples from New York state as of Jul. 2, 2021, and all SARS-CoV-2 wastewater samples as of Sep. 2021. Metadata tables for all processed SRAs are available at ://github.com/degregory/Cryptic_WNY_sup/tree/main/SRAs. Fastq files were processed similarly to our iSeq and MiSeq sequencing runs with the merging step skipped for unpaired reads. Reads mapped to the spike Orf were processed with SAM Refiner with the parameters “--wgs 1 -- min_count 1 --min_samp_abund 0”.

### Plasmids

Eukaryotic expression vectors for the heavy and light chains of antibodies LY-CoV016, LY-CoV555, and REGN10987 were obtained from Genscript. The lentiviral reporter constructed containing Gaussia luciferase (Gluc) with a reverse-intron (HIV-1-GLuc) was previously described49. The codon-optimized SARS-CoV-2 spike expression vector was obtained from Tom Gallagher50. This construct was modified to enhance transduction efficiency by truncating the last 19 amino acids and introducing the D614G amino acid change. DNA gBlocks containing the WNY RBD sequences were synthesized by IDT and introduced into the SARS-CoV-2 expression construct using In-Fusion cloning (Takara Bio, 638943). Lentiviral Mouse and Rat Ace2 vectors pscALPSpuro-MmACE2 (Mouse) and pscALPSpuro-RnACE2 (Rat) were obtained from Jeremy Luban51.

### Cell Culture

The 293FT cell line was obtained from Invitrogen. The 293FT+TMPRSS2 and 293FT+TMPRS S2+human Ace2 cells were previously described52. All cells were maintained in Dulbecco's modified Eagle's medium (DMEM, Cytiva, SH30022.01) supplemented with 10% fetal bovine serum, 2 mM L-glutamine (Sigma, G751), 1 mM sodium pyruvate (Sigma, S8636), 10 mM nonessential amino acids (Sigma, M7145), and 1% minimal essential medium (MEM) vitamins (Sigma, M6895). The ACE2 cell lines were generated by transfecting 293FT cells with 500 ng HIV GagPol expression vector, 400 ng of pscALPSpuro-MmACE2 (Mouse) or pscALPSpuro-RnACE2 (Rat), and 100 ng of VSV-G expression vector. Viral medium was used to transduce 293FT+TMPRSS2 cells53, and cells were selected with puromycin (1 mg/mL) (Sigma, P8833) beginning 2 days postransduction and were maintained until control treated cells were all eliminated.

### Monoclonal Antibody Synthesis

Transfections of 10 cm dishes of 293FT cells were performed with 5 μg each of heavy and light chain vectors and 40 μg polyethyleneimine (PEI) (Polysciences, 23966-2)53.

### Virus Production and Infectivity Assays

All transfections were performed in 10 cm dishes. 293FT cells were transfected with a total of 9 μg of HIV-1-GLuc, 1 mg of CMV spike vector, and 40 μg of PEI (Polysciences, 23966-2)53. Supernatants containing the virus were collected 2 days of post-transfection. Transduction of ACE2 expressing cells was performed by plating 30,000 cells in 96 well plates and co-culturing with 50 μL of HIV-1-GLuc/Spike particles. GLuc was measured 2 days post-transduction. All measurements were taken from distinct samples.

### Antibody Neutralization Assay

Subjects were requested to provide a date of positive PCR test for SARS-CoV-2 and subsequently had laboratory-based serologic tests to confirm the presence of antibody against SARS-CoV-2 51 RBD protein. A total of 10-20 mL of blood was collected from each participant. The plasma was then separated from the blood cells by centrifugation and stored at −80° C.

### Pseudovirus Neutralization Assay

All human plasma samples were heat inactivated for 30 min at 56° C. prior to the assay. Samples were diluted at 2-fold in ten serial dilutions in duplicates. Serially diluted samples were incubated with pre-titrated amounts of indicated pseudovirus at 37° C. for 1 h before addition of 293FT cells expressing human ACE2 and TMPRSS2 at 30,000 cells per well. Cells were incubated for 2 days and then the supernatant was used to measure Gaussia luciferase (RLU). All measurements were taken from distinct samples. Infection was normalized to the wells infected with pseudovirus alone.

### Statistical Analysis

Data and statistical analyses were performed in GraphPad Prism 9.0. A two-way ANOVA was performed to analyze the effect of receptor type and virus genotype on Gaussia luciferase intensity. Neutralization IC50 titers were calculated using nonlinear regression (inhibitor vs. normalized response—variable slope). Non-parametric pairwise analysis for neutralization titers were performed by Wilcoxon matched-pairs signed rank test.

### Reporting Summary

Further information on research design is available in the Nature Research Reporting Summary linked to this article.

### Data Availability

Source data are provided with this paper. Raw sequencing reads are available in NCBI's Sequence Read Archive (SRA) under accession #PRJNA715712.

### References

1. Pecson, B. M. et al. Reproducibility and sensitivity of 36 methods to quantify the SARS-CoV-2 genetic signal in raw wastewater: findings from an interlaboratory methods evaluation in the U.S. Environ. Sci. Water Res. Technol. 7, 504-520 (2021).
2. Trujillo, M. et al. Protocol for safe, affordable, and reproducible isolation and quantitation of SARS-CoV-2 RNA from wastewater. PLoS ONE 16, e0257454 (2021).
3. Peccia, J. et al. Measurement of SARS-CoV-2 RNA in wastewater tracks community infection dynamics. Nat. Biotechnol. 38, 1164 (2020).
4. Gonzalez, R. et al. COVID-19 surveillance in Southeastern Virginia using wastewater-based epidemiology. Water Res. 186, 116296 (2020).
5. Crits-Christoph, A. et al. Genome sequencing of sewage detects regionally prevalent SARS-CoV-2 variants. mBio 12, e02703-e02720 (2021).
6. Fontenele, R. S. et al. High-throughput sequencing of SARS-CoV-2 in wastewater provides insights into circulating variants. Water Res. 205, 117710 (2021).
7. Smyth, D. S. et al. Detection of mutations associated with variants of concern via high throughput sequencing of SARS-CoV-2 isolated from NYC wastewater. Preprint at medRxiv https://doi.org/10.1101/2021.03.21.21253978 (2021).
8. Gregory, D. A., Wieberg, C. G., Wenzel, J., Lin, C.-H. & Johnson, M. C. Monitoring SARS-CoV-2 populations in wastewater by amplicon sequencing and using the novel program SAM Refiner. Viruses 13, 1647 (2021).
9. Weisblum, Y. et al. Escape from neutralizing antibodies by SARS-CoV-2 spike protein variants. eLife 9, e61312 (2020).
10. Peacock, T. P., Penrice-Randal, R., Hiscox, J. A. & Barclay, W. S. SARS-CoV-2 one year on: evidence for ongoing viral adaptation. J. Gen. Virol. 102, 001584 (2021).
11. Wang, Z. et al. mRNA vaccine-elicited antibodies to SARS-CoV-2 and circulating variants. Nature 592, 616-622 (2021).
12. Starr, T. N., Greaney, A. J., Dingens, A. S. & Bloom, J. D. Complete map of SARS-CoV-2 RBD mutations that escape the monoclonal antibody LY-CoV555 and its cocktail with LY-CoV016. Cell Rep. Med. 2, 10025 (2021).
13. Starr, T. N. et al. Prospective mapping of viral mutations that escape antibodies used to treat COVID-19. Science 371, 850 (2021).
14. Liu, Z. et al. Identification of SARS-CoV-2 spike mutations that attenuate monoclonal and serum antibody neutralization. Cell Host Microbe 29, 477-488 (2021).
15. Huang, K. et al. Q493K and Q498H substitutions in spike promote adaptation of SARS-CoV-2 in mice. EBioMedicine 67, 103381 (2021).
16. Zhang, Y. et al. SARS-CoV-2 rapidly adapts in aged BALB/c mice and induces typical pneumonia. J. Virol. 95, e02477-20.
17. Dinnon, K. H. et al. A mouse-adapted model of SARS-CoV-2 to test COVID-19 countermeasures. Nature 586, 560-566 (2020).
18. Zhou, P. et al. A pneumonia outbreak associated with a new coronavirus of probable bat origin. Nature 579, 270-273 (2020).
19. Koley, T. et al. Structural analysis of COVID-19 spike protein in recognizing the ACE2 receptor of different mammalian species and its susceptibility to viral infection. 3 Biotech 11, 109-109 (2021).
20. Bao, L. et al. The pathogenicity of SARS-CoV-2 in hACE2 transgenic mice. Nature 583, 830-833 (2020).
21. Armero, A., Berthet, N. & Avarre, J.-C. Intra-host diversity of SARS-Cov-2 should not be neglected: case of the state of Victoria, Australia. Viruses 13, 133 (2021).
22. Lythgoe, K. A. et al. SARS-CoV-2 within-host diversity and transmission. Science 372, eabg0821 (2021).
23. Oreshkova, N. et al. SARS-CoV-2 infection in farmed minks, the Netherlands, April and May 2020. Euro Surveill. 25, 2001005 (2020).
24. McAloose, D. et al. From people to Panthera: Natural SARS-CoV-2 infection in tigers and lions at the Bronx zoo. mBio 11, e02220-e02220 (2020).
25. Patterson, E. I. et al. Evidence of exposure to SARS-CoV-2 in cats and dogs from households in Italy. Nat. Commun. 11, 6231 (2020).
26. Halfmann, P. J. et al. Transmission of SARS-CoV-2 in domestic cats. N. Engl. J. Med. 383, 592-594 (2020).
27. Montagutelli, X. et al. The B1.351 and P.1 variants extend SARS-CoV-2 host range to mice. Preprint at bioRxiv https://doi.org/10.1101/2021.03.18.436013 (2021).
28. Dróżdż, M. et al. Current state of knowledge about role of pets in zoonotic transmission of SARS-CoV-2. Viruses 13, 1149 (2021).
29. de Morais, H. A. et al. Natural infection by SARS-CoV-2 in companion animals: a review of case reports and current evidence of their role in the epidemiology of COVID-19. Front. Vet. Sci. 7, 823 (2020).
30. Cohen, J. From mice to monkeys, animals studied for coronavirus answers. Science 368, 221 (2020).
31. Spay and Neuter Practices among Cat Owners in New York City. https://a860-gpp.nyc.gov/concern/nyc_government_publications/mg74qm651 (2015).
32. Kilgour, R. J. et al. Estimating free-roaming cat populations and the effects of one year Trap-Neuter-Return management effort in a highly urban area. Urban Ecosyst. 20, 207-216 (2017).
33. NYC Dog Licensing Dataset. https://data.cityofnewyork.us/Health/NYC-Dog-Licensing-Dataset/nu7n-tubp (2017).
34. Auerbach, J. Does New York City really have as many rats as people? Significance 11, 22-27 (2014).
35. Barnes, C. O. et al. SARS-CoV-2 neutralizing antibody structures inform therapeutic strategies. Nature 588, 682-687 (2020).
36. Lilly's bamlanivimab (LY-CoV555) administered with etesevimab (LY-CoV016) receives FDA emergency use authorization for COVID-19. https://investor.lilly.com/news-rel eases/news-release-details/lillys-bamlanivimab-ly-cov555-administered-etesevimab-1y-cov016 (2021).
37. Jones, B. E. et al. The neutralizing antibody, LY-CoV555, protects against SARS-CoV-2 infection in nonhuman primates. Sci. Transl. Med. 13, eabf1906 (2021).
38. Baum, A. et al. Antibody cocktail to SARS-CoV-2 spike protein prevents rapid mutational escape seen with individual antibodies. Science 369, 1014 (2020).
39. Baaij ens, J. A., Stougie, L. & Schönhuth, A. Strain-aware assembly of genomes from mixed samples using flow variation graphs. Preprint at bioRxiv https://doi.org/10.1101/645721 (2020).
40. Robinson, C. A. et al. Defining biological and biophysical properties of SARS-CoV-2 genetic material in wastewater. Sci Total Environ. 807, 150786 (2021).
41. Chandler, J. C. et al. SARS-CoV-2 exposure in wild white-tailed deer (Odocoileus virginianus). Proc. Natl Acad. Sci. 118, e2114828118 (2021).
42. Miller, H. Geneious Knowledge Base. https://help.geneious.com/hc/en-us/articles/360045070991-Assembly-of-SARS-CoV-2-genomes-from-tiled-amplicon-Illumina-sequencing-using-Geneious-Prime (2021).
43. Posada-Cespedes, S. et al. V-pipe: a computational pipeline for assessing viral genetic diversity from high-throughput data. Bioinformatics 37, 1673-1680 (2021).
44. Rognes, T., Flouri, T., Nichols, B., Quince, C. & Mahé, F. VSEARCH: a versatile open-source tool for metagenomics. PeerJ 4, e2584 (2016).
45. Li, H. Minimap2: pairwise alignment for nucleotide sequences. Bioinformatics 34, 3094-3100 (2018).
46. Garrison, E. & Marth, G. Haplotype-based variant detection from short-read sequencing. https://arXiv.org/1207.3907v2 (2012).
47. Thorvaldsdóttir, H., Robinson, J. T. & Mesirov, J. P. Integrative Genomics Viewer (IGV): high-performance genomics data visualization and exploration. Brief Bioinform. 14, 178-192 (2013).
48. Langmead, B. & Salzberg, S. L. Fast gapped-read alignment with Bowtie 2. Nat. Methods 9, 357-359 (2012).
49. Janaka, S. K., Lucas, T. M. & Johnson, M. C. Sequences in gibbon ape leukemia virus envelope that confer sensitivity to HIV-1 accessory protein Vpu. J. Virol. 85, 11945-11954 (2011).
50. Qing, E., Hantak, M., Perlman, S. & Gallagher, T. Distinct roles for sialoside and protein receptors in coronavirus infection. mBio 11, e02764-19 (2020).
51. Yurkovetskiy, L. et al. Structural and functional analysis of the D614G SARS-CoV-2 Spike protein variant. Cell 183, 739-751 (2020).
52. Johnson, M. C. et al. Optimized pseudotyping conditions for the SARS-CoV-2 Spike glycoprotein. J. Virol. 94, e01062-20 (2020).
53. Boussif, O. et al. A versatile vector for gene and oligonucleotide transfer into cells in culture and in vivo: polyethylenimine. Proc. Natl Acad. Sci. 92, 7297 (1995).

### Example II: Genetic Diversity and Evolutionary Convergence of Cryptic SARS- CoV-2 Lineages Detected Via Wastewater Sequencing

Reference is made to Gregory D. et al. Genetic diversity and evolutionary convergence of cryptic SARS- CoV-2 lineages detected via wastewater sequencing. PLOS Pathogens 2022; and is incorporated in its entirety.

### Results

Beginning in early 2021, wastewater surveillance programs including RBD amplicon sequencing (FIG. 5A) were independently implemented in Missouri [9] and NYC [25]. A similar strategy was subsequently adopted in California by the University of California, Berkeley wastewater monitoring laboratory (COVID-WEB). All of the sequence output was analyzed with our previously described SAM Refiner pipeline [9], which is designed to remove PCR-generated chimeric sequences. While the vast majority of sequences observed with this method matched to known lineages identified in patients, reproducible lineages that did not match the known circulating lineages were also detected. Herein, we refer to each RBD haplotype with a unique combination of amino acid changes as a lineage, and combinations of lineages that all have specific amino acid changes in common as lineage classes. Amino acid combinations identified that have not been seen previously from patients are referred to as cryptic lineages. Here we describe cryptic lineages detected from Jan. 1, 2021 through Mar. 15, 2022.

For display purposes, for most sewersheds (those with >3 cryptic lineage-positive samples) individual polymorphisms were only displayed if they were present in at least two independent samples. Further, individual lineages were only displayed if they were over 2% of the total signal in at least one sample or were present in at least 2 independent samples. The detailed display criteria is outlined in Materials and Methods. The complete uncompressed data sets are included in S1-S9 Data of the incorporated manuscript Gregory D. et al. . PLOS Pathogens 2022.

### 2.1 Lineage Persistence and Evolution Over Time

In total, cryptic lineages were observed in 9 sewersheds across 3 states (Table 6) out of approximately 180 sewersheds that were routinely monitored. Each cryptic lineage class was generally unique to a sewershed. These lineages contained between 4-24 non-synonymous substitutions, insertions, and deletions. In some cases, lineages were detected for a short duration but with multiple similar co-occurring sequences. For example, in Missouri sewershed MO33, a lineage class containing 4-5 RBD amino acid changes were consistently detected at low relative abundances from March 15 to the end of April 2021 (FIG. 5B and Table 6). A total of 7 unique sequences were spread across the 5 sampling events in this date range, and multiple unique sequences co-occurred within a given sample.

TABLE 6
Overview of cryptic lineage detection

| | | Days | Number | Number |
|---|---|---|---|---|
| Loca- | Date range when | within | of | of RBD |
| tion | lineages appeared | range | samples | mutations |
| NY2 | Aug. 16, 2021-Feb. 28, 2022 | 170 | 10 | 4-18 |
| NY3 | Jan. 31, 2021 [8]-Mar. 14, 2022 | 437 | 7 | 16-24 |
| NY10 | Apr. 4, 2021-Nov. 29, 2021 | 239 | 22 | 4-11 |
| NY11 | Apr. 19, 2021-Nov. 22, 2022 | 217 | 20 | 4-9 |
| NY13 | Oct. 26, 2021-Feb. 14, 2022 | 111 | 5 | 12-15 |
| NY14 | May 10, 2021-Oct. 18, 2021 | 161 | 9 | 8-15 |
| MO33 | Mar. 15, 2021-Apr. 27, 2021 | 43 | 12 | 4-6 |
| MO45 | Jun. 8, 2021-Feb. 22, 2022 | 259 | 3 | 4-5 |
| CA | Nov. 4, 2021-Dec. 21, 2021 | 47 | 3 | 16 |

Meanwhile, in other sewersheds, cryptic lineages were detected briefly, before disappearing, and then reappearing many months later. For example, in Missouri sewershed MO45, lineages were first detected in June 2021 and then were not seen again until February 2022 (FIG. 5C and Table 6). The longest observed lineage class was in sewershed NY3 where we previously reported a lineage class from January 2021 [8] that was detected sporadically until March 2022 (FIG. 6A and Table 6). On average, cryptic lineages lasted for around 6 months, such as the lineage class from NY14 which lasted from May to October, 2021 (FIG. 6B and Table 6).

Each sewershed had its own unique set of lineages, but these lineages were not static. For instance, in NY10, the lineages first detected in April 2021 contained 4-5 RBD amino acid changes, but by October and November the lineages contained at least 6-8 RBD amino acid changes (FIG. 7 and Table 6).

In some cases, the sewersheds contained more than one lineage class. For instance, the NY11 sewershed contained several closely related lineages (class A) starting in April 2021, but a new lineage class (class B) was detected starting in August 2021. These two classes were clearly distinct with very few amino acid changes in common (FIG. 8A and Table 6).

Overall, specific lineage classes persisted within, but did not spread beyond, their individual sewersheds, with one notable exception. A cryptic lineage detected on Aug. 16, 2021 in NYC sewershed NY2 precisely matched a lineage detected in sewershed NY11 between June-September 2021 (FIG. 8A and 8B indicated by *). The NY11 and NY2 sewersheds do not border each other, but are not separated by any bodies of water.

In addition to amino acid changes, several of the lineages observed in these sewersheds contained amino acid deletions near positions 445 and 484. For instance, lineages NY11 contained 445-446 deletions, NY14 contained 444-445 deletions, NY3 and NY11 contained a deletion at position 484, and NY2 contained a deletion at position 483 (FIGS. 6 and 8).

Most cryptic lineages detected did not contain changes consistent with being derived from any known VOCs. The one exception was a lineage class containing amino acid changes N501Y and A570D in NY13 that first appeared on Sep. 26, 2021, which suggested possible derivation from the Alpha VOC (FIG. 9 and Table 6). The Alpha VOC had been the dominant lineage in NYC between April and June 2021, but by Sep. 26, 2021, it had been supplanted by Delta VOC and was no longer being detected in NYC [26].

### 2.2 Rare and Concerning Amino Acid Changes are Common in Cryptic Lineages and are Sometimes Shared with Omicron

In November 2021, the Omicron VOC was first detected in South Africa. This VOC contained eleven changes in the Spike protein between amino acids 410-510. Of these eleven amino acid changes, four (K417T, S477N, T478K, and N501Y) were present in previous VOCs. The remaining seven amino acid changes were rare prior to the Omicron VOC. All seven of these new amino acid changes had been detected in at least one of the wastewater lineages: N440K (MO33), G4465 (NY2), E484A (MO45, NY10, NY11, NY2, NY13, CA), Q493R (NY3, NY14), G4965 (NY2), Q498R (NY13, NY14), and Y505H (NY2, NY3, NY13, NY14, CA) (FIGS. 6 and 8-10). None of the wastewater lineages have combinations of amino acid changes consistent with having a common ancestor with Omicron and most were initially detected prior to the emergence of Omicron. However, these shared amino acid changes suggest that the cryptic lineages were under selective pressures similar to those that shaped the Omicron lineage.

Although each sewershed with cryptic lineages had its own signature combinations of amino acid changes, many of these changes were recurring among multiple sewersheds. Some of the more striking examples are described below.

N460K. All nine of the sewersheds contained lineages with this change. Changes at this position are known to lead to evasion of class I neutralizing antibodies [27,28]. However, this amino acid change was very rare, appearing in less than 0.01% of sequences in GISAID [22-24] submitted by Mar. 15, 2022 (Table 7).

TABLE 7
Table. Prevalence in GISAID of common substitutions found in cryptic lineages. Data reflects
the number of sequences from humans deposited into GISAID by the indicated dates¹

| | | Global Prevalence | Global |
|---|---|---|---|
| Substi- | | in Humans | Prevalence |
| tution | Location | Nov. 1, 2021 | Mar. 15, 2022 |
| Total Sequences | | 4,824,812 | 9,349,201 |
| G413R | NY10, NY14 | 147 | 239 |
| K417T | MO33, MO45, NY2, NY3, | 108,537 | 119,127 |
| | NY11, NY13, NY14, CA | | |
| N439K | MO33, NY2, NY3 | 37,227 | 40,274 |
| N440K | MO33 | 9,154 | 1,652,112 |
| K444Δ | NY14 | 14 | 68 |
| K444S | NY2, NY11 | 21 | 26 |
| K444T | NY2, NY10, NY11, NY14 | 58 | 217 |
| V445Δ | NY11, NY14 | 24 | 84 |
| V445A | CA, NY3, NY13, NY14 | 298 | 557 |
| G446S | CA, NY2 | 472 | 1,336,425 |
| G446Δ | NY11 | 19 | 82 |
| G446D | NY3, NY13 | 75 | 224 |
| Y449R | NY2, NY3, NY11 | 0 | 0 |
| L452Q | NY2, NY3 | 10,498 | 12,137 |
| L452R | MO33, NY3, NY14 | 2,315,718 | 4,324,990 |
| Y453F | NY3 | 1,320 | 1,497 |
| F456L | NY2, NY3, NY10, NY14 | 259 | 736 |
| N460K | NY2, CA, MO33, MO45, NY3, | 76 | 242 |
| | NY10, NY11, NY13, NY14 | | |
| S477N | CA, NY3, NY10, NY14 | 71,960 | 2,164,897 |
| T478K | CA, MO45, NY2, NY3 | 2,249,016 | 6,340,479 |
| V483Δ | NY2 | 49 | 932 |
| E484Δ | NY3, NY11 | 31 | 912 |
| E484A | CA, MO45, NY2, NY10, NY11, NY13 | 551 | 2,087,453 |
| E484P | NY2 | 0 | 73 |
| E484V | NY3, NY10 | 104 | 1,610 |
| F486P | CA, NY3, NY10, NY11 | 2 | 3 |
| F486V | NY13, NY14 | 4 | 34 |
| F490Y | CA, NY2, NY10, NY11, NY14 | 120 | 163 |
| Q493R | NY3, NY14 | 26 | 2,083,669 |
| Q493K | MO33, MO45, NY10, NY13, NY14 | 152 | 835 |
| S494P | MO45, NY2, NY10 | 12,916 | 15,009 |
| Q498H | CA, MO45, NY2, NY11, NY14 | 36 | 57 |
| Q498R | NY13 | 91 | 2,007,408 |
| Q498Y | NY2, CA, NY3, NY10, NY11, NY14 | 0 | 13 |
| P499S | CA, NY14 | 216 | 353 |
| N501S | CA, NY10, NY11, NY14 | 166 | 663 |
| N501T | CA, MO33, NY2, NY3, NY10, NY11, NY14 | 4,742 | 5,639 |
| N501Y | NY11, NY13 | 1,325,387 | 3,389,688 |
| G504D | NY3, NY14 | 190 | 580 |
| Y505H | CA, NY2, NY3, NY11, NY13, NY14 | 133 | 2,013,881t |
| H519N | NY10, NY11 | 13 | 31 |
| T572I | CA, NY2 | 16,610 | 26,948 |
| T572N | NY10, NY14 | 148 | 298 |

¹Khare S, Gurry C, Freitas L, Schultz M B, Bach G, Diallo A, et al. GISAID's Role in Pandemic Response. China CDC Wkly. 2021; 3: 1049-1051. doi: 10.46234/ccdcw2021.255

K417T. Eight of the nine sewersheds contained lineages with the amino acid change K417T. Changes at this position are common and are known to participate in evasion from class I neutralizing antibodies [27,28]. Although K417T was present in the Gamma VOC, K417N is the more common amino acid change at this position. The K417N amino acid change was not observed in any of the wastewater cryptic lineages.

N501S/T. The amino acid changes N501S and N501T were seen in four and seven of the nine sewersheds, respectively. Changes at this position directly affect receptor binding and can affect the binding of multiple classes of neutralizing antibodies [19,29,30]. Although mutations at this position are very common, the most common change by far is N501Y, which was present in multiple VOCs. By contrast, N501S and N501T were present in less than 0.01% and 0.1% of sequences in GISAID [22-24] submitted by Mar. 15, 2022 (Table 7).

Q498H/Y. Seven of the nine sewersheds in this study contained lineages with the amino acid change Q498H or Q498Y. It should be noted that Q498Y differs from the Wuhan ancestral sequence by two nucleotide substitutions at the 498th codon (CAA→TAC). Q498H (CAA→CAC) is a necessary intermediary in this transition as TAA encodes a stop codon. In several cases both Q498H and Q498Y were seen in association with particular lineage classes including in NY2, NY11, NY14 and CA (FIGS. 6B, 8 and 10). Changes at this position directly affect receptor binding [19,29,30]. Notably, Q498H and Q498Y have been associated with mouse adapted SARS-CoV-2 lineages [31-33]. Both of these amino acid changes are very rare, appearing in less than 0.01% of sequences in GISAID [22-24] submitted by Mar. 15, 2022. Prior to November 2021, Q498Y had never been seen in a patient sample (Table 7).

E484A. Six of the nine sewersheds contained lineages with the amino acid change E484A. Changes at this position are known to participate in evasion from class II neutralizing antibodies [27,28]. Prior to the emergence of Omicron in November 2021, E484A was present in about 0.01% of sequences submitted to GISAID [22-24] (Table 7).

Q493K. Five of the nine sewersheds contained lineages with the amino acid change Q493K. Changes at this position directly affect receptor binding and can affect the binding of multiple classes of neutralizing antibodies [19,27-30,34]. This amino acid change is biophysically very similar to the Q493R mutation in Omicron. However, the Q493K amino acid change was very rare in patient derived sequences, appearing in less than 0.01% of sequences in GISAID [22-24] submitted by March 15, 2022 (Table 7).

Y505H. Five of the nine sewersheds contained lineages with the amino acid change Y505H. Prior to the emergence of Omicron in November 2021, Y505H was present in about 0.01% of sequences submitted to GISAID [22-24] (Table 7).

K444T and K445A. The amino acid changes K444T and K445A were each seen in four of the nine sewersheds. Changes at these positions are known to participate in evasion from class III neutralizing antibodies [28]. However, these amino acid changes were very rare, each appearing in less than 0.01% of sequences in GISAID [22-24] submitted by Mar. 15, 2022 (Table 7).

Y449R. Three of the nine sewersheds contained lineages with the amino acid change Y449R. This change is noteworthy because, as of Mar. 15, 2022, no sequences with this amino acid change had been submitted to GISAID [22-24] (Table 7).

### 2.3 Long-Read Sequencing of S1 Identifies Substantial NTD Modifications and Suggests High dN/dS Ratio

With each sample that contained novel cryptic lineages, attempts were made to amplify a larger fragment of the S1 domain of Spike. Amplification of larger fragments from wastewater is often inefficient, but sometimes can be achieved. To gain more information about the S1 domain of Spike and independently confirm the authenticity of the RBD lineages, we optimized a PCR strategy that amplifies 1.6 kb of the SARS-COV-2 Spike encompassing amino acids 57-579. These fragments were then either subcloned and sequenced or directly sequenced using Pacific Biosciences HiFi sequencing (FIG. 11A).

The S1 amplification from the MO33 and MO45 sewersheds contained the RBD amino acid changes previously seen and each contained 3 additional amino acid changes upstream from the region sequenced using the targeted amplicon strategy described above (FIG. 11A). Many of the S1 amplifications from the NY10, NY11, NY13 and NY14 sewersheds contained numerous changes in S1 (FIG. 11A). In particular, many of the sequences contained deletions near amino acid positions 63-75, 144, and 245-248. All three of these areas are unstructured regions of the SARS-COV-2 spike where deletions have been commonly observed in sequences obtained from patients

[35]. Two distinct S1 sequences were detected from the NY14 sample collected on Jun. 28, 2021. Interestingly, the first sequence contained 13 amino acid changes which matched the RBD sequences from the same sewershed. The second sequence did not match any lineage that had been seen before, though it contained several mutations that were commonly seen in other cryptic lineages (see section 2.2). This second sequence presumably represented a unique lineage that had not been detected by routine wastewater surveillance.

A single S1 sequence was obtained from the NY13 samples collected on Oct. 31, 2021. This sequence generally matched the RBD sequence from the same date, but did contain minor variations. Importantly, the S1 sequence contained deletions at positions 69-70 and 144, which, along with the amino acid changes N501Y and A570D, match the changes found in the Alpha VOC lineage. This information is consistent with the NY13 lineages being derived from the Alpha VOC.

Comparing the number of non-synonymous to synonymous mutations in a sequence can elucidate the strength of positive selection imposed on a sequence. The ratios of non-synonymous and synonymous mutations in this region of S1 from the Alpha, Delta, and Omicron VOCs (BA.1) were 19/0, 2/0, and 4/1, respectively. It was not possible to calculate the formal dN/dS ratios since many of the sequences did not have synonymous mutations in this region, so instead the numbers of non-synonymous and synonymous mutations were plotted. The cryptic lineages contained 5 to total non-synonymous mutations and 0 to 2 total synonymous mutations (FIG. 11B).

### 2.4 Cryptic Lineages from NCBI Suggest an Early Common Ancestor for Many of the NYC Lineages

In addition to RBD amplicon sequencing performed in our laboratories, we downloaded the 5609 SARS-CoV-2 wastewater fastq files from NCBI's Sequence Read Archive (SRA) that were publicly available on NCBI on Jan. 21, 2022 (not including submissions from our own groups). We screened these sequences for cryptic lineages by searching for recurring amino acid changes seen via RBD amplicon sequencing (K444T, Y449R, N460K, E484A, F486P, Q493K, Q493R, Q498H, Q498Y, N501S, N501T, and Y505H) (see above and Table 7), requiring at least two of these mutations with a depth of at least 4 reads. This strategy identified samples from 15 sewersheds (Table 8). Four were collected from unknown sewersheds in New Jersey and California in January 2021. The other 11 were collected by the company Biobot from NYC between June and August 2021. All but one of the lineages closely matched the cryptic sequences that had been observed via RBD amplicon sequencing from the same sewershed. The one exception was SRR16038150, which contained 4 amino acid changes that had not been seen in any of the previous sewershed samples in the same combination. The Biobot sequences were 40-96% complete and appeared to contain 30-100% cryptic lineages based on the frequency of mutation A23056C (Q498H/Y), a mutation shared with the lineages in all 11 sewershed samples from NYC. We speculate that the relative abundance of cryptic lineages was high because, during this period, NYC experienced the lowest levels of COVID-19 infections seen since the start of the pandemic, and therefore the level of patient-derived SARS-CoV-2 RNA in wastewater was very low. As a result, the sequences that matched the known circulating lineage were at low abundance.

TABLE 8
Cryptic lineage whole genome sequences from nationwide surveys.

| | | | | Percentage | | | | |
|---|---|---|---|---|---|---|---|---|
| SRA | | | | cryptic | Genome | | PANGO | |
| Accession | State | Submitter | Sample Date | lineage | coverage | Sewershed | assignment | RBD Changes |
| SRR17120725 | CA | Aquavitas | 2021 Jan. 4 | 7% | 27,403 | n/a^{a} | ND^{b} | E484A/Q498H/ |
| | | | | | | | | H519N |
| SRR16638981 | NJ | Aquavitas | 2021 Jan. 18 | 7% | 28,185 | n/a^{a} | ND^{b} | E484A/Q498H/ |
| | | | | | | | | H519N |
| SRR16542155 | NJ | Aquavitas | 2021 Jan. 18 | 7% | 27,295 | n/a^{a} | ND^{b} | E484A/Q498H/ |
| | | | | | | | | H519N |
| SRR16362183 | NJ | Aquavitas | 2021 Jan. 4 | 100% | 15,217 | n/a^{a} | ND^{b} | E484A/Q498H/ |
| | | | | | | | | H519N |
| SRR16038150 | NY | Biobot | 2021 Aug. 17 | 79% | 28,227 | NY2 | B.1.503 | Y449P/E484A/ |
| | | Analytics | | | | | | F490Y/Q498H |
| SRR16038156 | NY | Biobot | 2021 Aug. 9 | 92% | 24,595 | NY11 | B.1.503 | K417T/K444T/ |
| | | Analytics | | | | | | Y449H/N460K/ |
| | | | | | | | | E484A/F490Y/ |
| | | | | | | | | Q498H |
| SRR15706711 | NY | Biobot | 2021 Aug. 9 | 100% | 11,877 | NY11 | ND^{b} | K417T/K444T/ |
| | | Analytics | | | | | | Y449H/N460K/ |
| | | | | | | | | E484A/F490Y/ |
| | | | | | | | | Q498H/A570D |
| SRR15384049 | NY | Biobot | 2021 Jul. 12 | 99% | 24,001 | NY10 | B.1 | Q493K/Q498Y/ |
| | | Analytics | | | | | | H519N/T572N) |
| SRR15291305 | NY | Biobot | 2021 Jul. 5 | 100% | 22,316 | NY11 | P.1.15 | K417T/K444T/ |
| | | Analytics | | | | | | Y449H/E484A/ |
| | | | | | | | | F490Y/Q498H |
| SRR15291304 | NY | Biobot | 2021 Jul. 4 | 100% | 28,634 | NY10 | B.1 | Q493K/Q498/ |
| | | Analytics | | | | | | H519N/T572N |
| SRR15202285 | NY | Biobot | 2021 Jun. 28 | 100% | 12,209 | NY2 | ND^{b} | K444S/V445K/ |
| | | Analytics | | | | | | G446V/Y449R/ |
| | | | | | | | | L452Q/N460K/ |
| | | | | | | | | K462R/ |
| | | | | | | | | S477N/T478E/ |
| | | | | | | | | T478R/DEL483/ |
| | | | | | | | | E484P/F486I/ |
| | | | | | | | | F490P/ |
| | | | | | | | | G496S/Q498Y/ |
| | | | | | | | | P499S/N501T/ |
| | | | | | | | | Y505H/V511I |
| SRR15202284 | NY | Biobot | 2021 Jun. 28 | 98 | 16,281 | NY14 | ND^{b} | K417T/K444S/ |
| | | Analytics | | | | | | DEL445-6/ |
| | | | | | | | | L452R/N460K/ |
| | | | | | | | | S477D/F486V/ |
| | | | | | | | | Q493K/Q498Y/ |
| | | | | | | | | P499S/N501T |
| SRR15202279 | NY | Biobot | 2021 Jun. 28 | 30% | 21,974 | NY11 | B.1 | N440K/K444S/ |
| | | Analytics | | | | | | DEL445-6/ |
| | | | | | | | | L452Q/Y453F/ |
| | | | | | | | | N460K/S477N/ |
| | | | | | | | | D484/F486A/ |
| | | | | | | | | Q493K/Q498K/ |
| | | | | | | | | P499S/N501Y/ |
| | | | | | | | | H519N |
| SRR15128983 | NY | Biobot | 2021 Jun. 16 | 99% | 21,152 | NY11 | A.29 | K444T/Y449H/ |
| | | Analytics | | | | | | E484A/Y489Y/ |
| | | | | | | | | F490Y/Q498H |
| SRR15128978 | NY | Biobot | 2021 Jun. 16 | 100% | 15,593 | NY10 | ND^{b} | E484A/F486P/ |
| | | Analytics | | | | | | S494/Q498Y/ |
| | | | | | | | | H519N |

To compare the mutational profile among these different NYC samples, we first determined all of the mutations that occurred in at least 3 of the 11 cryptic lineages. We then produced a heat map to compare the frequency of each of these mutations from wastewater samples with the mutations that were reported from New York patient samples in June 2021 (FIG. 12). Surprisingly, the sewershed sequences often lacked two of the four consensus sequences that define the B.1 PANGO lineage (GISAID G clades or Nextstrain ‘20’ clades) of SARS-COV-2 [36]. Almost all patient samples collected in NYC during June 2020 contained the mutations C241T, C3037T, C14408T, and A23403G. The cryptic lineages from NYC wastewater all appeared to contain the mutations C3037T and A23403G, but possessed the ancestral sequences at positions 241 and 14408. In addition, there were two mutations in the S gene that were found in nearly all of the cryptic lineages, A23056C (Q498H/Y) and C24044T (L828F). Both of these mutations were found in less than 1% of patient samples. There were 3 additional mutations outside of the S gene that were highly prevalent in most of the wastewater samples, but essentially absent from patient samples: C25936G (Orf3 H182D), G25947C (Orf3 Q185H), and T27322C (Orf6 S41P). While other mutations were detected repeatedly within a sewershed, no other mutations spanned multiple sewersheds.

To confirm that some of the cryptic lineages lacked the B.1 lineage consensus mutations, we designed primers to amplify and sequence the C14408 region of SARS-CoV-2 RNA isolated from wastewater. Indeed, samples from NY11 and NY10 that had a high prevalence of cryptic lineages were found to contain sequences that lacked C14408T (FIG. 13). However, when samples were amplified from the NY13 sewershed when the cryptic lineages there were present, we observed only the modern C14408T, as would be expected if the NY13 lineage were derived from the Alpha VOC. In addition, we performed whole genome sequencing on a Mar. 30, 2021 sample from MO33 when the cryptic lineages were highly prevalent and did not detect any sequence that lacked C241T or C14408T, suggesting the cryptic lineages in this sewershed diverged after the emergence of the B.1 lineage. Finally, we also analyzed the sequences from NCBI that contained the cryptic lineages from NJ and CA and did not find any sequences lacking C241T or C14408T. Thus, the lineages lacking C241T and C14408T appear to be limited to a subset of the cryptic lineages from NYC. These data are consistent with the hypothesis that a SARS-CoV-2 lineage bearing mutations C3037T and A23403G, but possessing the ancestral genotype at positions 241 and 14408, was the direct ancestor of most of the cryptic lineages found in NYC.

### 3. Discussion

Our results point to the evolution of numerous SARS-CoV-2 lineages under positive immune selection whose source/host remains unknown.

### 3.1 Relatedness of and Origin of Cryptic Lineages

We previously detected cryptic lineages via targeted amplicon sequencing [8], but lacked information about their derivation. Here, from comparison of the sewersheds for which whole genome sequencing is available, it is clear that the cryptic lineages from wastewater are not all derived from a common ancestor. The NY13 lineage appeared to be derived from the Alpha VOC. If this is true, the NY13 lineage most likely branched off from Alpha sometime in early to mid-2021 when that variant was common in NYC. However, many lineages from the NY10, NY11, NY2, and NY14 sewersheds in New York appear to likely share a common ancestor that branched off from a pre-B.1 lineage. Additionally, we often observed swarms of related sequences that co-occurred within a sewershed on a single date, and accumulated new mutations over time, suggesting continued diversification from a single origin within each sewershed.

### 3.2 Comparison with the Omicron VOC

The Omicron VOC and the wastewater lineages appear to have been subjected to high positive selection. While prior VOCs had 3 or fewer amino acid changes in the amplified region of the RBD, the Omicron VOC (BA.1) contained 11 and the cryptic lineages from wastewater averaged over 10. By comparison, a cluster of SARS-COV-2 sequences that appear to have circulated in white-tailed deer for over a year accumulated only 2 amino acid changes in this region [37]. Of the nonsynonymous RBD mutations in Omicron, four were in at least one prior VOC: K417N, S477N, T478K, and N501Y. The other seven were relatively rare; N440K was present in of sequences and the other six were each present in less than 0.1% of sequences in GISAID [22-24] prior to Nov. 1, 2021. All of the rare Omicron changes were observed in at least one of the cryptic wastewater lineages. Collectively, this suggests that the wastewater lineages and the Omicron VOC likely arose under similar selective pressures. The high dN/dS ratios found in cryptic lineages and in Omicron suggest that these selective pressures must be exceptionally strong.

### 3.3 Source of Lineages

In spite of detailed tracking and cataloging of the cryptic lineages, the question where they are coming from remains unanswered. The most parsimonious explanations are 1) undetected spread within the human population, 2) prolonged shedding by individuals, or 3) spread in animal reservoirs.

Undetected spread in the population appears unlikely. While the sequencing rate for US patient samples is not 100%, it is high enough that population-level spread of cryptic lineages would not be missed. Alternatively, as it is known that SARS-CoV-2 can replicate in gastrointestinal sites [38,39], the lack of detection of cryptic lineages by clinical sequencing could be explained by the potential adaptation of some SARS-CoV-2 to replicate exclusively in the gastrointestinal tract [1,38]. Nonetheless, even if replication of these lineages were occurring outside of the nasopharyngeal region, this could not explain why cryptic lineages generally remain geographically constrained.

The most likely explanation for the appearance of cryptic lineages in wastewater is that they are shed by people with long-term COVID infections. Many such infections have been documented, particularly in immunosuppressed populations. Indeed, the vast majority of amino acid changes in the RBD of the Omicron VOC and the cryptic lineages confer resistance to neutralizing antibodies. In particular, substitutions at positions 417, 440, 460, 484, 493 and 501 have all been well documented to lead to immune evasion [17,27,34,40-42]. Additionally, RBD changes K417T, N440K, N460K, E484A, Q493K, and N501Y have all been observed in persistent infections of immunocompromised patients [43,44]. Given the repeated appearance of these mutations in diverse sewersheds, the majority of the selective pressure on the cryptic lineages is almost certainly immune pressure. A possible explanation for cryptic lineages is that they are the result of long-term SARS-CoV-2 infections of intestinal tissue. A recent paper reported extended presence of viral RNA in feces, long after it was undetectable in respiratory samples and suggested SARS-CoV-2 replication in the gastrointestinal (GI) tract could explain some of the symptoms

associated to long-Covid [38]. The authors propose that SARS-CoV-2 infects the gastrointestinal tract and that some individuals shed the virus up to 7-months post-diagnosis.

The counterargument to cryptic lineages coming from patients is the sheer volume of viral shedding required to account for the wastewater signal. Many of the sewersheds process 50-100 million gallons of wastewater per day. Reliable amplification of a sequence from wastewater generally requires that the sequence is present at least 10,000 copies per liter. Therefore, detection of a specific virus lineage in such a sewershed would seem to require several trillion virus particles to be deposited each day. If this signal were derived from a single infected patient or even a small group of patients, those patients would have to shed exponentially more virus than typical COVID-19 patients.

The final explanation for the cryptic lineages in wastewater is that they are shed into wastewater by an animal host population. Previously, we determined through rRNA analysis of several NYC sewersheds that the major non-human mammals that contribute to the wastewater are cats, rats, and dogs [8]. Of these three, rats were the only species that seemed to be a plausible candidate. Indeed, we also showed that the cryptic lineages from the sewersheds had the ability to utilize rat and mouse ACE2 [8]. However, one of the sewersheds with the most consistent signal in 2021 was NY10, which had little to no rat rRNA. In addition, it is not clear why circulation in an immune competent animal such as a dog or a rat would result in a more rapid selection of immune escape mutations than circulation in humans, yet the cryptic lineages display accumulation of many times more immune escape changes than seen in viruses circulating in the human population.

### 3.4 The Importance of Wastewater Sequencing Methodology for Identification of Novel Variants

To provide information regarding the appearance and spread of SARS-CoV-2 variants in communities, next generation sequencing technologies have been applied to sequence SARS-CoV-2 genetic material obtained from sewersheds around the world [45-47]. Commonly, SARS-CoV-2 RNA extracted from wastewater is amplified using SARS-CoV-2 specific primers that cover the entire genome [48-50]. Bioinformatic pipelines are employed to identify circulating SARS-CoV-2 variants [16,51]. In general, the presence and abundance of variants in wastewater corresponds to data obtained from clinical sequencing [45,46]. However, to our knowledge, there have been no other reports of cryptic lineages detected in wastewater that were not also observed in clinical sequence data. A major issue with generating whole genome sequence data from nucleic acid isolated from wastewater is sequence dropout over diagnostically important regions of the genome [48,52,53]. In some cases, diagnostically important regions of the genome that accumulate many mutations, such as the Spike RBD, receive little to no sequence coverage, making variant attribution difficult. Since wastewater contains a mixture of virus lineages and whole genome sequencing relies on sequencing of small genome fragments, mutations appearing on different reads cannot be linked together. Indeed, some variant identification pipelines map reads to

reference genomes to estimate the probability that mutations are found in the same genome [16]. Such strategies would not be able to detect variants containing unique constellations of mutations. Detecting novel variants that are present at low relative abundances may be better achieved by targeted amplicon sequencing, such as the strategy we present here.

### Methods

### 4.1 Wastewater Sample Processing and RNA Extraction

24-hr composite samples of wastewater were collected weekly from the inflow at each of the wastewater treatment plans.

NYC: Samples were processed on the day they were collected and RNA was isolated according to our previously published protocol [6]. Briefly, 250 mL from a 24-hr composite wastewater sample from each WWTP were centrifuged at 5,000×g for 10 min at 4° C. to pellet solids. A 40 mL aliquot from the centrifuged samples was passed through a 0.22 μM filter (Millipore). To each corresponding filtrate, 0.9 g sodium chloride and 4.0 g PEG 8000 (Fisher Scientific) were added. The tubes were kept at 4° C. for 24 hrs and then centrifuged at 12,000×g for 120 minutes at 4° C. to pellet the precipitate. The pellet was resuspended in 1.5 mL TRIzol (Fisher Scientific), and RNA was purified according to the manufacturer's instructions.
MO: Samples were processed as previously described [9]. Briefly, wastewater samples were centrifuged at 3000×g for 10 min and then filtered through a 0.22 μM polyethersulfone membrane (Millipore, Burlington, MA, USA). Approximately 37.5 mL of wastewater was mixed with 12.5 mL solution containing 50% (w/vol) polyethylene glycol 8000 and 1.2 M NaCl, mixed, and incubated at 4° C. for at least 1 h. Samples were then centrifuged at 12,000×g for 2 h at 4° C. Supernatant was decanted and RNA was extracted from the remaining pellet (usually not visible) with the QIAamp Viral RNA Mini Kit (Qiagen, Germantown, MD, USA) using the manufacturer's instructions. RNA was extracted in a final volume of 60 μL.
CA: Samples were processed as previously described [54]. Briefly, 40 mLs of influent was mixed with 9.35 g NaCl and 400 uL of 1M Tris pH 7.2, 100 mM EDTA. Solution was filtered through a 5-um PVDF filter and 40 mLs of 70% EtNY11 was added. Mixture was passed through a silica spin column. Columns were washed with 5 mL of wash buffer 1 (1.5 M NaCl, 10 mM Tris pH 7.2, 20% EtNY11), and then 10 mL of wash buffer 2 (100 mM NaCl, 10 mM Tris pH 7.2, 80% EtNY11). RNA was eluted with 200 ul of ZymoPURE elution buffer.

### 4.2 Targeted PCR: MiSeq Sequencing

The primary RBD RT-PCR was performed using the Superscript IV One-Step RT-PCR System (Thermo Fisher Scientific,12594100). Primary RT-PCR amplification was performed as follows: 25° C. (2:00)+50° C. (20:00)+95° C. (2:00)+[95° C. (0:15)+55° C. (0:30)+72° C. (1:00)]×25 cycles using the MiSeq primary PCR primers CTGCTTTACTAATGTCTATGCAGATTC SEQ ID NO: 6 and NCCTGATAAAGAACAGCAACCT SEQ ID NO: 14. Secondary PCR (25 μL) was performed on RBD amplifications using 5 μL of the primary PCR as template with MiSeq nested gene specific primers containing 5′ adapter sequences (0.5 μM each) acactctttccctacacgacgctcttccgatctGTRATGAAGTCAGMCAAATYGC SEQ ID NO: 15 and gtgactggagttcagacgtgtgctcttccgatctATGTCAAGAATCTCAAGTGTCTG SEQ ID NO: 9, dNTPs (100 μM each) (New England Biolabs, N0447L) and Q5 DNA polymerase (New England Biolabs, M0541S). Secondary PCR amplification was performed as follows: 95° C. (2:00)+[95° C. (0:15)+55° C. (0:30)+72° C. (1:00)]×20 cycles. A tertiary PCR (50 μL) was performed to add adapter sequences required for Illumina cluster generation with forward and reverse primers (0.2 μM each), dNTPs (200 μM each) (New England Biolabs, N0447L) and Phusion High-Fidelity or (KAPA HiFi for CA samples) DNA Polymerase (1U) (New England Biolabs, M0530L). PCR amplification was performed as follows: 98° C. (3:00)+[98° C. (0:15)+50° C. (0:30)+72° C. (0:30)]×7 cycles+72° C. (7:00). Amplified product (10 μl ) from each PCR reaction is combined and thoroughly mixed to make a single pool. Pooled amplicons were purified by addition of Axygen AxyPrep MagPCR Clean-up beads (Axygen, MAG-PCR-CL-50) or in a 1.0 ratio to purify final amplicons. The final amplicon library pool was evaluated using the Agilent Fragment Analyzer automated electrophoresis system, quantified using the Qubit HS dsDNA assay (Invitrogen), and diluted according to Illumina's standard protocol. The Illumina MiSeq instrument was used to generate paired-end 300 base pair reads. Adapter sequences were trimmed from output sequences using Cutadapt.

### 4.3 Long PCR and Subcloning

The long RBD RT-PCR was performed using the Superscript IV One-Step RT-PCR System (Thermo Fisher Scientific, 12594100). Primary long RT-PCR amplification was performed as follows: 25° C. (2:00)+50° C. (20:00)+95° C. (2:00)+[95° C. (0:15)+55° C. (0:30)+72° C. (1:30)]×25 cycles using primary primers CCCTGCATACACTAATTCTTTCAC SEQ ID NO: 16 and TCCTGATAAAGAACAGCAACCT SEQ ID NO:7. Secondary PCR (25 μL) was performed on RBD amplifications using 5 μL of the primary PCR as template with nested primers (0.5 μM each) CATTCAACTCAGGACTTGTTCTT SEQ ID NO: 17 and ATGTCAAGAATCTCAAGTGTCTG SEQ ID NO: 18 , dNTPs (100 μM each) (New England Biolabs, N0447L) and Q5 High-Fidelity DNA Polymerase (New England Biolabs, M0491L). Secondary PCR amplification was performed as follows: 95° C. (2:00)+[95° C. (0:15)+55° C. (0:30)+72° C. (1:30)]×20 cycles.

Positive amplifications were visualized in an agarose gel stained with ethidium bromide, excised, and purified with a NucleoSpin Gel and PCR Clean-up Kit (Macherey-Nagel, 74609.250). Gel purified DNA was subcloned using a Zero Blunt TOPO PCR Cloning Kit (Invitrogen, K2800-20SC). Individual colonies were transferred to capped test tubes containing 10 ml of 2×YT broth (ThermoFisher, BP9743-5). Test tubes were incubated at 37° C. and shook at 250 rpm for 24 hours. The resulting E. Coli colonies were centrifuged for 10 minutes at 5000×g and the supernatant was decanted. Plasmid DNA was extracted from the pellet using a GeneJet Plasmid Miniprep Kit (ThermoFisher, K0503). The concentration of plasmid DNA extracts was measured using a NanoDrop One (ThermoFisher, ND-ONE-W).

### 4.4 PacBio Sequencing

A nested RT-PCR protocol was used to generate 1.6 kb Spike amplicons from wastewater RNAs for PacBio sequencing. The primary RT-PCR amplification was performed with the Superscript IV One-Step RT-PCR System (Invitrogen) and the same thermal cycling program as described above for MiSeq amplicons. These inter Spike gene-specific primer sequences (5′-[BC10ab]-ATTCAACTCAGGACTTGTTCTT SEQ ID NO: 19 and 5′-[BC10xy]-ATGTCAAGAATCTCAAGTGTCTG SEQ ID NO: 18) were tagged directly on their 5′ ends with standard 16 bp PacBio barcode sequences and used with asymmetric barcode combinations that allow large numbers of samples to be pooled prior to sequencing. The following thermal cycling profile was used for nested PCR: 98° C. (2 min)+[98° C. (10 sec)+55° C.(10 sec)+72° C. (1 min)]×20 cycles+72° C. (5 min). The resulting PCR amplicons were then subjected to three rounds of purification with AMPure XP beads (Beckman Coulter Life Sciences) in a ratio of 0.7:1 beads to PCR. Purified amplicons were quantified using a Qubit dsDNA HS kit (ThermoFisher Scientific) and pooled prior to PacBio library preparation.

After ligation of SMRTbell adaptors according to the manufacturer's protocol, sequencing was completed on a PacBio Sequel II instrument (PacBio, Menlo Park, CA USA) in the Genomic Sequencing Laboratory at the Centers of Disease Control in Atlanta, GA, USA. Raw sequence data was processed using the SMRT Link v10.2 command line toolset.Circular consensus sequences were demultiplexed based on the asymmetric barcode combinations and subjected to PB Amplicon Analysis to obtain high-quality consensus sequences and search for minor sequence variants.

### 4.5 Bioinformatics

### 4.5.1 MiSeq and PacBio Processing

Sequencing reads were processed as previously described. Briefly, VSEARCH tools were

used to merge paired reads and dereplicate sequences [55]. Dereplicated sequences from RBD amplicons were mapped to the reference sequence of SARS-CoV-2 (NC_045512.2) spike ORF

using Minimap2 [56]. Mapped amplicon sequences were then processed with SAM Refiner using the same spike sequence as a reference and the command line parameters “—Alpha 1.8—foldab 0.6” [9].

The covariant deconvolution outputs were used to generate the haplotype plots in FIGS. 5-11. Covar outputs of SAM Refiner for MiSeq sequences were collected by sewershed and multiple runs of the same sample averaged. The collected sequence data were processed to determine core haplotypes of cryptic lineages observed in each sewershed. First sequences that contained only one or no variation relative to the reference Wuhan I sequence were discarded. Remaining sequences with 6 or fewer variations and containing the polymorphisms defining Alpha, Beta, Gamma or Delta were assigned to the defining haplotype of the matching VOC. Any sequences not reassigned with fewer than 4 variations were removed. Sequences with at least 6 variations were matched against Omicron BA.1, BA.2 and BA.5. Sequences that matched an Omicron lineage with more than 70% identity were assigned to the defining haplotype for the matching lineage. Remaining unassigned sequences were then processed to remove polymorphisms that did not appear in at least two sample dates (except for MO45 and California sequences, due to the small number of samples with cryptic sequences) or never appeared in a sample at an abundance greater than 0.5%. In-frame deletions bypassed this removal. Condensed sequences that appear in at least two samples or had a summed abundance of at least 2% across all samples were passed on to further steps. The above process was reiterated until no more processing occurred. Non-VOC sequences were then aligned via MAFFT and then all sequences rendered into figures using plotnine. The PacBio sequences were similarly collected to generate the haplotype plot in FIG. 11, without the polymorphism condensation or alignment.

### 4.5.2 NCBI SRA Screening

Raw reads were downloaded and then processed similar to MiSeq sequencing except the reads were mapped to the entire SARS-CoV-2 genome and SAM Refiner was run with the parameters ‘—wgs 1—collect 0—indel 0—covar 0—min count 1—min_samp_abund 0—min_col_abund 0—ntabund 0—ntcover 1’. Unique sequence outputs from SAM Refiner were then screened for specific amino acid changes. The nt call outputs of samples of interest were used to determine other variations in the genomes sequenced.

### 4.5.3 14408 Sequencing

The long RBD RT-PCR was performed using the Superscript IV One-Step RT-PCR System (Thermo Fisher Scientific, 12594100). Primary long RT-PCR amplification was performed as follows: 25° C. (2:00)+50° C. (20:00)+95° C. (2:00)+[95° C. (0:15)+55° C. (0:30)+72° C. (1:30)]×25 cycles using primary primers ATACAAACCACGCCAGGTAG SEQ ID NO: 20 and AACCCTTAGACACAGCAAAGT SEQ ID NO: 21. Secondary PCR (25 μL) was performed on RBD amplifications using 5 μL of the primary PCR as template with nested primers (0.5 μM each) ACACTCTTTCCCTACACGACGCTCTTCCGATCTGGTAGTGGAGTTCCTGTTGTAG SEQ ID 22 NO: and GTGACTGGAGTTCAGACGTGTGCTCTTCCGATCTAGCACGTAGTGCGTTTATCT SEQ ID NO: 23, dNTPs (100 μM each) (New England Biolabs, N0447L) and Q5 High-Fidelity DNA Polymerase (New England Biolabs, M0491L). Secondary PCR amplification was performed as follows: 95° C. (2:00)+[95° C. (0:15)+55° C. (0:30)+72° C. (1:30)]×20 cycles.

### 4.5.2 Whole Genome Sequencing

Whole genome sequencing of the SARS-CoV-2 genome from the MO33 sewershed was performed using the NEBNext ARTIC SARS-CoV-2 Library Prep Kit (Illumina). Amplicons were sequenced on an Illumina MiSeq instrument. Output sequences were analyzed using the program SAM Refiner [58].

### References

1. Cheung K S, Hung I F N, Chan P P Y, Lung K C, Tso E, Liu R, et al. Gastrointestinal

Manifestations of SARS-CoV-2 Infection and Virus Load in Fecal Samples From a Hong Kong Cohort: Systematic Review and Meta-analysis. Gastroenterology. 2020; 159: 81-95. pmid:32251668
1. Parasa S, Desai M, Thoguluva Chandrasekar V, Patel H K, Kennedy K F, Roesch T, et al.

Prevalence of Gastrointestinal Symptoms and Fecal Viral Shedding in Patients With Coronavirus Disease 2019: A Systematic Review and Meta-analysis. JAMA Netw Open. 2020; 3: e2011335. pmid:32525549
2. Ahmed W, Tscharke B, Bertsch PM, Bibby K, Bivins A, Choi P, et al. SARS-CoV-2 RNA monitoring in wastewater as a potential early warning system for COVID-19 transmission in the community: A temporal case study. Sci Total Environ. 2021;761: 144216. pmid:33360129
3. Gonzalez R, Curtis K, Bivins A, Bibby K, Weir M H, Yetka K, et al. COVID-19 surveillance in Southeastern Virginia using wastewater-based epidemiology. Water Res. 2020; 186: 116296. pmid:32841929
4. Hoar C, Chauvin F, Clare A, McGibbon H, Castro E, Patinella S, et al. Monitoring SARS-CoV-2 in wastewater during New York City's second wave of COVID-19: Sewershed-level trends and relationships to publicly available clinical testing data. medRxiv. 2022; 2022.02.08.22270666.
5. Trujillo M, Cheung K, Gao A, Hoxie I, Kannoly S, Kubota N, et al. Protocol for Safe,

Affordable, and Reproducible Isolation and Quantitation of SARS-CoV-2 RNA from Wastewater. medRxiv. 2021; 2021.02.16.
6. Kirby A E, Welsh R M, Marsh Z A, Yu A T, Vugia D J, Boehm A B, et al. Notes from the Field: Early Evidence of the SARS-CoV-2 B.1.1.529 (Omicron) Variant in Community Wastewater—United States, November-December 2021. MMWR Morb Mortal Wkly Rep. 2022; 71: 103-105. pmid:35051130
7. Smyth D S, Trujillo M, Gregory D A, Cheung K, Gao A, Graham M, et al. Tracking cryptic SARS-CoV-2 lineages detected in NYC wastewater. Nat Commun. 2022; 13: 635. pmid:35115523
8. Gregory D A, Wieberg C G, Wenzel J, Lin C-H, Johnson M C. Monitoring SARS-CoV-2 Populations in Wastewater by Amplicon Sequencing and Using the Novel Program SAM Refiner. Viruses. 2021; 13. pmid:34452511
9. Martin D P, Weaver S, Tegally H, San J E, Shank S D, Wilkinson E, et al. The emergence and ongoing convergent evolution of the SARS-CoV-2 N501Y lineages. Cell. 2021/09/07 ed. 2021; 184: 5189-5200.e7. pmid:34537136
10. Callaway E. BEYOND OMICRON: WHAT'S NEXT FOR SARS-COV-2 EVOLUTION. NATURE. 2021; 600: 204-207.
11. Hill V, Du Plessis L, Peacock T P, Aggarwal D, Colquhoun R, Carabelli A M, et al. The origins and molecular evolution of SARS-CoV-2 lineage B.1.1.7 in the UK. bioRxiv. 2022; 2022.03.08.481609.
12. swift C L, Isanovic M, Correa Velez K E, Norman R S. Community-level SARS-CoV-2 sequence diversity revealed by wastewater sampling. Sci Total Environ. 2021/08/18 ed. 2021; 801: 149691-149691. pmid:34438144
13. Herold M, d′Herouel A F, May P, Delogu F, Wienecke-Baldacchino A, Tapp J, et al. Genome

Sequencing of SARS-CoV-2 Allows Monitoring of Variants of Concern through Wastewater. Water. 2021; 13.
14. Fontenele R S, Kraberger S, Hadfield J, Driver E M, Bowes D, Holland L A, et al. High-throughput sequencing of SARS-CoV-2 in wastewater provides insights into circulating variants. medRxiv. 2021; 2021.01.22.21250320. pmid:33501452
15. Baaijens J A, Zulli A, Ott I M, Petrone M E, Alpert T, Fauver J R, et al. Variant abundance estimation for SARS-CoV-2 in wastewater using RNA-Seq quantification. medRxiv. 2021; 2021.08.31.21262938. pmid:34494031
16. Greaney A J, Starr T N, Barnes C O, Weisblum Y, Schmidt F, Caskey M, et al. Mapping mutations to the SARS-CoV-2 RBD that escape binding by different classes of antibodies. Nat Commun. 2021; 12: 4196. pmid:34234131
17. Harvey W T, Carabelli A M, Jackson B, Gupta R K, Thomson E C, Harrison E M, et al. SARS-CoV-2 variants, spike mutations and immune escape. Nat Rev Microbiol. 2021; 19: 409-424. pmid:34075212
18. Shang J, Ye G, Shi K, Wan Y, Luo C, Aihara H, et al. Structural basis of receptor recognition by SARS-CoV-2. Nature. 2020; 581: 221-224. pmid:32225175
19. Liu H, Wei P, Kappler J W, Marrack P, Zhang G. SARS-CoV-2 Variants of Concern and Variants of Interest Receptor Binding Domain Mutations and Virus Infectivity. Front Immunol. 2022; 13. pmid:35154144
20. Li Q, Wu J, Nie J, Zhang L, Hao H, Liu S, et al. The Impact of Mutations in SARS-CoV-2

Spike on Viral Infectivity and Antigenicity. Cell. 2020/07/17 ed. 2020; 182: 1284-1294.e9. pmid:32730807
21. Khare S, Gurry C, Freitas L, Schultz M B, Bach G, Diallo A, et al. GISAID's Role in Pandemic

Response. China CDC Wkly. 2021; 3: 1049-1051. pmid:34934514
22. Elbe S, Buckland-Merrett G. Data, disease and diplomacy: GISAID's innovative contribution to global health. Glob Chall Hoboken NJ. 2017; 1: 33-46. pmid:31565258
23. Shu Y, McCauley J. GISAID: Global initiative on sharing all influenza data—from vision to reality. Euro Surveill Bull Eur Sur Mal Transm Eur Commun Dis Bull. 2017; 22: 30494. pmid:28382917
24. Smyth D S, Trujillo M, Cheung K, Gao A, Hoxie I, Kannoly S, et al. Detection of Mutations

Associated with Variants of Concern Via High Throughput Sequencing of SARS-CoV-2 Isolated from NYC Wastewater. medRxiv. 2021; 2021.03.21.21253978.
25. nychealth/coronavirus-data. NYC Department of Health and Mental Hygiene;
26. Starr T N, Greaney A J, Dingens A S, Bloom J D. Complete map of SARS-CoV-2 RBD mutations that escape the monoclonal antibody LY-CoV555 and its cocktail with LY-CoV016. Cell Rep Med. 2021; 2: 100255. pmid:33842902
27. Starr T N, Greaney A J, Addetia A, Hannon W W, Choudhary M C, Dingens A S, et al. Prospective mapping of viral mutations that escape antibodies used to treat COVID-19. Science. 2021; 371: 850-854. pmid:33495308
28. Starr T N, Greaney A J, Hilton S K, Ellis D, Crawford K H D, Dingens A S, et al. Deep Mutational

Scanning of SARS-CoV-2 Receptor Binding Domain Reveals Constraints on Folding and ACE2 Binding. Cell. 2020; 182: 1295-1310.e20. pmid:32841599
29. Lan J, Ge J, Yu J, Shan S, Zhou H, Fan S, et al. Structure of the SARS-CoV-2 spike receptor-binding domain bound to the ACE2 receptor. Nature. 2020; 581: 215-220. pmid:32225176
30. Dinnon K H, Leist S R, Schafer A, Edwards C E, Martinez D R, Montgomery S A, et al. A mouse-adapted model of SARS-CoV-2 to test COVID-19 countermeasures. Nature. 2020; 586: 560-566. pmid:32854108
31. Wang J, Shuai L, Wang C, Liu R, He X, Zhang X, et al. Mouse-adapted SARS-CoV-2 replicates efficiently in the upper and lower respiratory tract of BALB/c and C57BL/6J mice. Protein Cell. 2020; 11: 776-782. pmid:32749592
32. Gawish R, Starkl P, Pimenov L, Hladik A, Lakovits K, Oberndorfer F, et al. ACE2 is the critical in vivo receptor for SARS-CoV-2 in a novel COVID-19 mouse model with TNF- and IFNγ-driven immunopathology. eLife. 2022; 11: e74623. pmid:35023830
33. Weisblum Y, Schmidt F, Zhang F, DaSilva J, Poston D, Lorenzi J C, et al. Escape from neutralizing antibodies by SARS-CoV-2 spike protein variants. eLife. 2020; 9: e61312. pmid:33112236
34. McCarthy Kevin R., Rennick Linda J., Nambulli Sham, Robinson-McCarthy Lindsey R., Bain William G., Haidar Ghady, et al. Recurrent deletions in the SARS-CoV-2 spike glycoprotein drive antibody escape. Science. 2021; 371: 1139-1142. pmid:33536258
35. Korber B, Fischer W M, Gnanakaran S, Yoon H, Theiler J, Abfalterer W, et al. Tracking Changes in SARS-CoV-2 Spike: Evidence that D614G Increases Infectivity of the COVID-19 Virus. Cell. 2020/07/03 ed. 2020; 182: 812-827.e19. pmid:32697968
36. Pickering B, Lung O, Maguire F, Kruczkiewicz P, Kotwa J D, Buchanan T, et al. Highly divergent white-tailed deer SARS-CoV-2 with potential deer-to-human transmission. Microbiology; 2022 February
37. Natarajan A, Zlitni S, Brooks E F, Vance S E, Dahlen A, Hedlin H, et al. Gastrointestinal symptoms and fecal shedding of SARS-CoV-2 RNA suggest prolonged gastrointestinal infection. Med. 2022; 52666634022001672. pmid:35434682
38. Zollner A, Koch R, Jukic A, Pfister A, Meyer M, Rössler A, et al. Postacute COVID-19 is Characterized by Gut Viral Antigen Persistence in Inflammatory Bowel Diseases. Gastroenterology. 2022; 50016508522004504. pmid:35508284
39. Greaney A J, Starr T N, Barnes C O, Weisblum Y, Schmidt F, Caskey M, et al. Mutational escape from the polyclonal antibody response to SARS-CoV-2 infection is largely shaped by a single class of antibodies. Microbiology; 2021 March pmid:33758856
40. Greaney A J, Loes A N, Crawford K H D, Starr T N, Malone K D, Chu H Y, et al. Comprehensive mapping of mutations in the SARS-CoV-2 receptor-binding domain that affect recognition by polyclonal human plasma antibodies. Cell Host Microbe. 2021; 29: 463-476.e6. pmid:33592168
41. Liu Z, VanBlargan L A, Bloyet L-M, Rothlauf P W, Chen R E, Stumpf S, et al. Identification of SARS-CoV-2 spike mutations that attenuate monoclonal and serum antibody neutralization. Cell Host Microbe. 2021; 29: 477-488.e4. pmid:33535027
42. Coronavirus Antiviral & Resistance Database. Stanford University;
43. Wilkinson S A, Richter A, Casey A, Osman H, Mirza J D, Stockton J, et al. Recurrent SARS-CoV-2 Mutations in Immunodeficient Patients. medRxiv. 2022; 2022.03.02.22271697. pmid:35996593
44. Crits-Christoph A, Kantor R S, Olm M R, Whitney O N, Al-Shayeb B, Lou Y C, et al. Genome Sequencing of Sewage Detects Regionally Prevalent SARS-CoV-2 Variants. Pettigrew M M, editor. mBio. 2021; 12: e02703-20. pmid:33468686
45. Fontenele R S, Kraberger S, Hadfield J, Driver E M, Bowes D, Holland L A, et al. High-throughput sequencing of SARS-CoV-2 in wastewater provides insights into circulating variants. Water Res. 2021; 205: 117710.
46. Izquierdo-Lara R, Elsinga G, Heijnen L, Munnink B B O, Schapendonk C M E, Nieuwenhuijse D, et al. Monitoring SARS-CoV-2 Circulation and Diversity through Community Wastewater Sequencing, the Netherlands and Belgium. Emerg Infect Dis. 2021; 27: 1405-1415. pmid:33900177
47. Cotten M, Lule Bugembe D, Kaleebu P, V T Phan M. Alternate primers for whole-genome SARS-CoV-2 sequencing. Virus Evol. 2021; 7: veab006-veab006. pmid:33841912
48. Xiao M, Liu X, Ji J, Li M, Li J, Yang L, et al. Multiple approaches for massively parallel sequencing of SARS-CoV-2 genomes directly from clinical samples. Genome Med. 2020; 12: 57. pmid:32605661
49. Amin Addetia, Lin Michelle J., Peddu Vikas, Roychoudhury Pavitra, Jerome Keith R., Greninger Alexander L., et al. Sensitive Recovery of Complete SARS-CoV-2 Genomes from Clinical Samples by Use of Swift Biosciences' SARS-CoV-2 Multiplex Amplicon Sequencing Panel. J Clin Microbiol. 59: e02226-20. pmid:33046529
50. Dezordi F Z, Neto A M da S, Campos T de L, Jeronimo P M C, Aksenen C F, Almeida S P, et al. ViralFlow: A Versatile Automated Workflow for SARS-CoV-2 Genome Assembly, Lineage Assignment, Mutations and Intrahost Variant Detection. Viruses. 2022; 14: 217. pmid:35215811
51. Van Poelvoorde L A E, Delcourt T, Coucke W, Herman P, De Keersmaecker S C J, Saelens X, et al. Strategy and Performance Evaluation of Low-Frequency Variant Calling for SARS-CoV-2 Using Targeted Deep Illumina Sequencing. Front Microbiol. 2021; 12.
52. Lin X, Glier M, Kuchinski K, Ross-Van Mierlo T, McVea D, Tyson J R, et al. Assessing Multiplex Tiling PCR Sequencing Approaches for Detecting Genomic Variants of SARS-CoV-2 in Municipal Wastewater. mSystems. 2021/10/19 ed. 2021; 6: e0106821-e0106821. pmid:34665013
53. N Whitney O, Al-Shayeb B, Crits-Cristoph A, Chaplin M, Fan V, Greenwald H, et al. V.4 -Direct wastewater RNA capture and purification via the &#34; Sewage, Salt, Silica and SARS-CoV-2 (45)&#34; method v4. 2020 Nov.
54. Rognes T, Flouri T, Nichols B, Quince C, Mahé F. VSEARCH: a versatile open source tool for metagenomics. PeerJ. 2016; 4: e2584. pmid:27781170
55. Li H. Minimap2: pairwise alignment for nucleotide sequences. Birol I, editor. Bioinformatics. 2018; 34: 3094-3100. pmid:29750242
56. Katoh K. MAFFT: a novel method for rapid multiple sequence alignment based on fast Fourier transform. Nucleic Acids Res. 2002; 30: 3059-3066. pmid:12136088
57. Gregory D A, Wieberg C G, Wenzel J, Lin C-H, Johnson M C. Monitoring SARS-CoV-2 Populations in Wastewater by Amplicon Sequencing and Using the Novel Program SAM Refiner. Viruses. 2021; 13: 1647. pmid:34452511.

### Example III: Tracing the origin of SARS-CoV-2 Omicron-Like Spike Sequences Detected in Wastewater

Reference is made to Shafer M. et al. Tracing the origin of SARS-CoV-2 Omicron-like spike sequences detected in wastewater. medRxiv 2022.10.28.22281553; and is incorporated in its entirety.

### Methods

### Collection of Wastewater

Wastewater samples for this study (January 2022 through March 2023) were collected in collaboration with experienced wastewater engineers from the city wastewater utility. The Wisconsin State Laboratory of Hygiene (WSLH) determined specific locations in the wastewater collection system to obtain samples for each round of testing, allowing them to gradually narrow down the origin of the Wisconsin Lineage source region. Sewage lift-stations, manholes, and facility sewer line access points were sampled with compositing autosamplers (ISCO 6712 and 6712c). Depending upon manhole depth, the autosampler was either placed on a shelf adjacent to the wastestream or suspended from the manhole opening, with weighted collection lines placed into the wastewater stream. The autosamplers were programmed to collect 24-hr composites, typically on a time-based mode, with wastewater composited into a 10-liter polypropylene container. The composite was kept cool during collection with ice packed around the collection container. Composite samples were transported to the analytical laboratory within a few hours of sample retrieval. While wastewater flows were available from the pump-stations and central municipal wastewater treatment facility, flow measurements were not made in the manhole waste streams.

### Isolation of Viral RNA from Wastewater

Wastewater samples were shared between the Wisconsin State Laboratory of Hygiene (WSLH) and the University of Missouri, with the WSLH focusing on virus quantitation and whole genome sequencing, and the University of Missouri focusing on RBD-targeted sequencing. At the WSLH, after the addition of a bovine coronavirus (BCoV) viral recovery control and concentration of virus using Nanotrap Magnetic Virus Particles (Ceres Nanosciences, VA, USA) on a Kingfisher Apex instrument (ThermoFisher Scientific, MA, USA), total nucleic acids were extracted using Maxwell(R) HT Environmental TNA kits (Promega, Madison, WI, USA) on a Kingfisher Flex instrument (ThermoFisher Scientific, Waltham, MA, USA). The University of Missouri concentrated the virus using a PEG protocol on pre-filtered samples (0.22 μM polyethersulfone membrane (Millipore, Burlington, MA, USA)).

Samples were incubated with PEG (polyethylene glycol 8000) and 1.2 M NaCl, centrifuged, and the RNA was isolated from the pellet with the QIAamp Viral RNA Mini Kit (Qiagen, Germantown, MD, USA).

Two approaches were used to isolate viral RNA from wastewater. For samples processed at WSLH, wastewater samples (homogenized and unfiltered) were spiked with 20 μL/250 mL Calf-Guard® (Zoetis, Parsippany, NJ, USA), a cattle vaccine containing Bovine Coronavirus (BCoV) (as a virus recovery control), and briefly stored at 4° C. until the viral targets were isolated and concentrated, typically on the day of receipt. A total of 10 mL (2×5 mL) of wastewater was concentrated using Nanotrap Magnetic Virus Particles, Microbiome A and Enhancing Reagent 2 (Ceres Nanosciences, Manassas, VA, USA), using a KingFisher Apex automation platform. Total nucleic acids (TNA) were extracted using Maxwell(R) HT Environmental TNA kits (Promega, Madison, WI, USA) and eluted in 200 μL of 25 mM Tris HCl (pH 8.0) buffer. The extraction was automated using a KingFisher Flex (ThermoFisher Scientific, Waltham, MA, USA). The long program was used for the concentration.

For samples processed at the University of Missouri, samples were processed as previously described.4 Briefly, wastewater samples were centrifuged at 3000×g for 10 min and filtered through a 0.22 μM polyethersulfone membrane (Millipore, Burlington, MA, USA). Approximately 37.5 mL of wastewater was mixed with 12.5 mL solution containing 50% (w/vol) polyethylene glycol 8000 and 1.2 M NaCl, mixed, and incubated at 4 C for at least 1 h. Samples were then centrifuged at 12,000×g for 2 h at 4 C. Supernatant was decanted and RNA was extracted from the remaining pellet (usually not visible) with the QIAamp Viral RNA Mini Kit (Qiagen, Germantown, MD, USA) using the manufacturer's instructions. RNA was extracted in a final volume of 60 μL.

### Quantification of Viral RNA by RT-dPCR

The WSLH quantified the concentration of SARS-CoV-2, BCoV (viral recovery control), and PMMoV (fecal marker) in each sample using reverse transcriptase digital PCR (RT-dPCR). PCR inhibition was probed with a bovine respiratory syncytial virus (BRSV) spiked into each PCR reaction.

Quantification of SARS-CoV-2, BCoV (internal control), PMMoV (fecal marker), and BRSV (spiked inhibition control) was achieved using reverse transcriptase digital PCR (RT-dPCR). Master mix was prepared using the One-Step Viral PCR kit (4×) (Qiagen, Germantown, MD, USA) and GT dPCR SARS-CoV-2 Wastewater Surveillance Assay Kit (GT Molecular, Fort Collins, CO, USA) with quantification of the following viral targets: N1, N2, BCoV, and PMMoV included with the GTMolecular dPCR SARS-CoV-2 Wastewater Surveillance Assay Kit, and BRSV primers and probes from IDT.5 The samples were quantified on a QIAcuity Four Digital PCR System (Qiagen, Germantown, MD, USA). N1, N2, and BCoV were multiplexed on QIAcuity Nanoplate 26k 24-well plates while PMA/IoV and BRSV were singleplexed on 8.5k 96-well nanoplates. Cycling and exposure conditions are detailed in the table shown below. Analysis of the RT-dPCR results was performed with the QIAcuity Software Suite version 2.1.7.182. Thresholds were manually set to separate negative and positive partitions.

TABLE 10
dPCR thermocycling conditions
Thermocycling Conditions:

| | Step | | Time | | Temp ° C. |
|---|---|---|---|---|---|
| Reverse Transcription | | | 30 | min | 50 |
| DNA polymerase activation | | | 2 | min | 95 |
| | 45 cycles | Denaturation | 10 | sec | 95 |
| | | Anneal/Extend | 30 | sec | 55 |

| Target | Channel | Exposure | Gain |
|---|---|---|---|
| N1 | Red (ROX) | 500 | 4 |
| N2 | Green (FAM) | 300 | 6 |
| BCoV | Yellow (HEX) | 300 | 6 |
| PMMoV | Green (FAM) | 300 | 6 |
| BRSV | Yellow (HEX) | 500 | 6 |

### Identification of Cryptic Lineages in Wastewater with Non-Omicron PCR Amplification and Amplicon Sequencing

A nested RT-PCR approach was used to selectively amplify non-Omicron spike protein RBD regions from wastewater samples. Amplified RBD regions were then sequenced using an Illumina MiSeq instrument and analyzed using the SAMRefiner software.19 The Wisconsin Lineage's unique RBD sequences were used to identify and track the lineage across time and space.

The primary RBD RT-PCR was performed using the Superscript IV One-Step RT-PCR System (Thermo Fisher Scientific, 12594100). Primary RT-PCR amplification was performed as follows: 25° C. (2:00)+50° C. (20:00)+95° C. (2:00)+[95° C. (0:15)+55° C. (0:30)+72° C. (1:00)]×25 cycles using the MiSeq primary PCR primers 5′-ATTCTGTCCTATATAATTCCGCAT-3′ SEQ ID NO: 24 and 5′-CCCTGATAAAGAACAGCAACCT-3′ SEQ ID NO: 25 (the first primer was changed to 5′-TATATAATTCCGCATCATTTTCCAC-3′ SEQ ID NO: 26 starting in May, 2022 to adapt to changing Omicron lineages). Secondary PCR (25 μL) was performed on RBD amplifications using 5 μL of the primary PCR as template with MiSeq nested gene specific primers containing 5′ adapter sequences (0.5 μM each) 5′-acactctttccctacacgacgctcttccgatctGTGATGAAGTCAGACAAATCGC-3′ SEQ ID NO: 27 and 5′-gtgactggagttcagacgtgtgctcttccgatctATGTCAAGAATCTCAAGTGTCTG-3′ SEQ ID NO: 9, dNTPs (100 μM each) (New England Biolabs, N0447L) and Q5 DNA polymerase (New England Biolabs, M0541S). Secondary PCR amplification was performed as follows: 95° C. (2:00)+[95° C. (0:15)+55° C. (0:30)+72° C. (1:00)]×20 cycles. A tertiary PCR (50 μL) was performed to add adapter sequences required for Illumina cluster generation with forward and reverse primers (0.2 μM each), dNTPs (200 μM each) (New England Biolabs, N0447L) and Phusion High-Fidelity or (KAPA HiFi for CA samples) DNA Polymerase (1U) (New England Biolabs, M0530L). PCR amplification was performed as follows: 98° C. (3:00)+[98° C. (0:15)+50° C. (0:30)+72° C. (0:30)]×7 cycles+72° C. (7:00). Amplified product (10 μl) from each PCR reaction is combined and thoroughly mixed to make a single pool. Pooled amplicons were purified by the addition of Axygen AxyPrep MagPCR Clean-up beads (Axygen, MAG-PCR-CL-50) or in a 1.0 ratio to purify final amplicons. The final amplicon library pool was evaluated using the Agilent Fragment Analyzer automated electrophoresis system, quantified using the Qubit HS dsDNA assay (Invitrogen), and diluted according to Illumina's standard protocol. The Illumina MiSeq instrument was used to generate paired-end 300 base pair reads. Adapter sequences were trimmed from output sequences using Cutadapt.

Sequencing reads were processed as previously described. Briefly, VSEARCH tools were used to merge paired reads and dereplicate sequences.6 Dereplicated sequences from RBD amplicons were mapped to the reference sequence of SARS-CoV-2 (NC_045512.2) spike ORF using Minimap2.7 Mapped amplicon sequences were then processed with SAM Refiner using the same spike sequence as a reference and the command line parameters “--Alpha 1.8 --foldab 0.6”.8

The haplotypes representing at least 25% of the total sequences in at least one sample were rendered into figures using plotnine

### SARS-CoV-2 Whole Genome Sequencing of Wastewater

For SARS-CoV-2 whole genome sequencing (WGS), 13 μL of total nucleic acids from the wastewater extracts were used as input to QIAGEN's Direct SARS-CoV-2 Enhancer kit (Qiagen, Germantown, MD, USA). Amplicon libraries were prepared on a Biomek i5 liquid handler (Beckman Coulter, Brea, CA, USA). Libraries were quantified using a High Sensitivity Qubit 1×dsDNA HS Assay Kit (ThermoFisher Scientific), and fragment size was analyzed by a QIAxcel Advanced and the QX DNA Screening Kit (QIAGEN, Germantown, MD, USA). Sequencing was performed on an Illumina MiSeq instrument using MiSeq Reagent v2 (300 cycles) kits. Fastq files were analyzed with the nf-core/viralrecon 2.5 workflow²⁰ (10.5281/zenodo.3901628) using the SARS-CoV-2 Wuhan-Hu-1 reference genome (Genbank accession MN908947.3) The workflow was initiated as outlined on the project's data portal (https://go.wisc.edu/4134pl).

Sequencing libraries were generated at the WSLH using the QIAseq DIRECT SARS-CoV-2 Enhanced kits with the primer Booster (QIAGEN, Germantown, MD, USA) following manufacturer's instructions. Briefly, 13 uL of total nucleic acid were reverse transcribed into cDNA using hexaprimers. SARS-CoV-2 genome was then specifically enriched using a SARS-CoV-2 primer panel. The panel consists of approximately 550 primers for creating 425 amplicons, covering the entire SARS-CoV-2 viral genome. UDI were 1:5 diluted. The library preparation was fully automated using the Biomek i5 Automated Workstation (Beckman Coulter). Libraries were quantified using a High Sensitivity Qubit 1×dsDNA HS Assay Kit (ThermoFisher Scientific) and fragment size analyzed by a QIAxcel Advanced and the QX DNA Screening Kit (QIAGEN, Germantown, MD, USA). Libraries were sequenced on an Illumina MiSeq platform using MiSeq Reagent v2 (300 cycles) kits.

Isolated RNA from each Facility Line B time point was whole-genome sequenced at least twice in separate Illumina MiSeq runs in anticipation of needing sequence technical replicates for later analysis. The data were analyzed with the nf-core/viralrecon workflow using the SARS-CoV-2 Wuhan-Hu-1 reference genome (Genbank accession MN908947.3) and the QIAseq Direct SARS-CoV-2 primer .bed file. After creating a sample sheet as described on the nf-core/viralrecon website the workflow was initiated as outlined on the project's data portal. The output “variants_long_table.csv” from iVar was made into a pivot table in Microsoft Excel to make Supplemental Table 2 (as disclosed in Shafer M. et al. medRxiv 2022.10.28.22281553). Because called variant frequencies differ between sequencing replicates from each time point, we decided to display the results from each replicate for the sake of transparency. Codons with variants detected in at least one sequence replicate from each time point were selected from Supplemental Table 2 and sorted by gene and frequency to make Supplemental Table 3 (as disclosed in Shafer M. et al. medRxiv 2022.10.28.22281553. The presence of a particular called variant in one sequence replicate indicates that that variant could be present in the sample. The absence of a called variant in a replicate, on the other hand, does not prove its absence from the sample. Thus, we decided to include variants in Supplemental Table 3 even if they were only present in one sequence replicate for each time point.

### Virus Culture

To remove debris, samples were centrifuged twice at 3,500 rpm at 4° C. for 15 minutes and then passed through a 0.8 μM syringe filter (Agilent) or left unfiltered. Samples (1 ml) were incubated on nearly confluent Vero E6-TMPRSS2 (JCRB1819) or Vero E6-TMPRSS2/hACE2 cells (from Barney Graham, NIH) seeded the day prior in TC252 cm flasks for 1 hour at 370 C. After the incubation, cells were washed twice and media was added back to the cells. The media contained 2-times the normal concentration of penicillin, streptomycin and amphotericin along with chloramphenicol. Cells were monitored daily for potential virus-induced cytopathic effects. After 10 days, a blind passage was performed using the entire volume of media (˜4 ml) to fresh, nearly confluent cells seeded the day prior in TC1752 cm flasks.

### Variant Proportion Assessment

Variant proportions were assessed from WGS data using Freyja v.1.3.11, a tool previously developed to estimate the proportions of SARS-CoV-2 variants in deep sequence data containing mixed populations (10.1038/s41586-022-05049-6). Briefly, BAM files generated using viralrecon were processed by Freyja to create the variant and depth files (Wuhan-Hu-1 reference genome: MN908947.3). Variant proportions were assessed utilizing the median estimates obtained via the Freyja bootstrap boot function (nb=10). The UShER barcode was updated on Mar. 20, 2023.

### Root-to-Tip Regression

To generate FIG. 17A, we first downloaded from GenBank all full consensus genomes for SARS-CoV-2 belonging to Pango lineage B.1.234 (the inferred parent of the Wisconsin Lineage) and collected from specimens in the Midwest region (Illinois, Indiana, Iowa, Kansas, Michigan, Minnesota, Missouri, Nebraska, North Dakota, Ohio, South Dakota, and Wisconsin). The accession numbers for this dataset can be found on the GitHub repository accompanying this repository. The dataset is composed of 304 individual genome sequences collected between 2020 May 4 and 2021May 1, which represents all the available B.1.234 sequences for the Midwest region available on GenBank. The dataset was filtered to exclude incomplete and low-quality sequences and to retain no more than 50 isolates per state. The list of accession numbers for the filtered isolates can also be found on the GitHub repository accompanying this manuscript. A total of 268 sequences were ultimately aligned to the Wuhan-Hu-1 reference sequence MN908947.3 using MAFFT (v7.505). A maximum likelihood phylogenetic tree was inferred using iqtree (v.2.2.0.3) with a molecular clock and distances obtained through treetime (v0.9.3). The analysis was conducted independently for the wastewater samples (WSLH-222, WSLH-223, WSLH-230, and WSLH-231) and root-to-tip distances for all strains were visualized in R (ggplot, dplyr). Phylogeny was visualized and annotated with FigTree (v.1.4.4). Scripts are available in the GitHub repository accompanying this manuscript (https://github.com/tcflab/wisconsin_cryptic_lineages).

### Analyses for Natural Selection

Variants obtained through the nf-core/viralrecon workflows were processed using custom Python scripts to generate panels b-d in FIG. 17. The multiple replicates for each collection date were used to obtain the intersection of variants, that is, variants that were found in all replicates for each collection date. The frequencies and depth of the resulting variants were recalculated. Variants differing from reference sequence Wuhan-Hu-1 (MN908947.3) were classified as non-synonymous (Non-syn), synonymous (Syn), insertions-deletions (indels), or others (including nonsense and frameshift mutations) using SnpEff (v.5.0). Synonymous and non-synonymous point mutations were quantified and compared between timepoints, and 95% confidence intervals obtained from the relative risk (RR) of every nucleotide substitution against its inverted change (i.e., RR=‘A>C’/‘C>A’) using SciPy's relative_risk function (v.1.9.3). To obtain the proportion of variants per site, we enumerated synonymous and non-synonymous substitutions across the SARS-CoV-2 genome, and obtained the proportion against the number of synonymous and non-synonymous sites, respectively, using SNPGenie (v.2019.10.31). A binomial probability distribution was implemented to obtain the 95% confidence intervals via SciPy's binomtest function (v.1.9.3). A Mann-Whitney two-sided test was applied to test the difference between πN and πS on each gene, while a one-sided test was used to test for an enrichment of the πN value of Spike against the πN value on the other genes. To obtain synonymous and nonsynonymous divergence values (panel e), the average Hamming distance between B.1.234 isolates (dataset used in FIG. 17a) and the MN908947.3 reference sequence was calculated as has been done previously for other coronaviruses.⁹ Divergence was obtained over a sliding window of 36 days by dividing the observed synonymous and nonsynonymous differences between the isolate and reference by the total possible number of synonymous and nonsynonymous nucleotide substitutions. Only windows that contained at least 2 sequences were considered for the analysis. Divergence values were independently calculated for each of the wastewater timepoints against the MN908947.3 reference sequence. Plot was visualized using Matplotlib. Scripts are available in the GitHub repository accompanying this manuscript (https://github.com/tcflab/wisconsin_cryptic_lineages).

### Results

On Jan. 11, 2022, a cryptic lineage containing at least six unusual spike RBD substitutions was first detected in a composite wastewater sample from a metropolitan publicly owned treatment works (POTW) in Wisconsin (FIG. 14c). This initial sample was composited from raw influent from five interceptor districts in the metropolitan area sewershed, effectively sampling a population of more than 100,000 people. The source of the enigmatic RBD sequences was narrowed by testing each of the five interceptor district lines. The Wisconsin Lineage was only detected in one district line, which served seven sub-districts. Of the seven sub-district lines, only the line from Sub-District 5 contained the target (FIG. 14c). Additional testing in March 2022 of manholes upstream of the interceptor line within Sub-District 5 confirmed the persistence of the Wisconsin Lineage and further refined the lineage's source. As the sampling effort progressed further upstream in the sewershed, the proportion of the Wisconsin Lineage (labeled B.1.234 in FIG. 14), determined using Freyja²¹ v.1.3.11, increased relative to the total SARS-CoV-2 sequences detected at each sampling site (FIG. 14b). By May 2022, this investigation had traced the source of the lineage to a single manhole accessing a lateral that collected wastewater from a single building. Subsequent testing of wastewater from sewer service lines within this building in June 2022 further narrowed the source to one sewer line serving six toilets (called “Facility Line B”) on one side of the building (FIG. 14a). 12s rRNA sequencing detected predominantly human rRNA from this source, supporting the hypothesis that this cryptic lineage was being shed by a human. Chicken rRNA, the next largest taxon identified, was less than 0.05% of the sample (Table 9). Facility Line B was retested for the cryptic lineage in August and again in September of 2022, remaining positive at each time point.

TABLE 9
Abundance of contributing species to the June Facility Line
B sample. 12S ribosomal RNA (rRNA) sequencing was performed
on two replicates of the June sample of Facility Line B. The
average count and abundance between these two samples is shown.
Homo sapiens (human) was found to be the predominant
contributor to this sample. Cow and chicken rRNA were the
second and third most abundant (<1% each).

| | | Facility Line B (June) | |
|---|---|---|---|
| | | Average | Average |
| Species | Common Name | Count | Abundance |
| Homo sapiens | Human | 116,842.68 | 88.22% |
| Unmatched | Unmatched | 12,522.80 | 9.46% |
| Pan paniscus | Human, chimp, but more | 4,507.00 | 3.40% |
| | likely human | | |
| Poor match | | 794.00 | 0.60% |
| Elephantulus | Human - mismatch, human | 72.00 | 0.05% |
| fuscipes | | | |
| Gallus gallus | Bird, Chicken | 49.00 | 0.04% |
| Bos taurus | Cattle | 30.00 | 0.02% |
| Pan troglodytes | Human, chimp, but also | 29.00 | 0.02% |
| | human | | |

As quantified by digital PCR, unprecedentedly high wastewater SARS-CoV-2 RNA viral loads were observed in samples collected from Facility Line B on June 16th (˜520,000,000 genome copies per liter undiluted wastewater), August 16th (˜1,600,000,000 copies per liter), September 23rd (˜2,700,000,000 copies per liter) and September 27th (˜550,000,000 copies per liter) (FIG. 14b). Drops of this raw wastewater tested positive for SARS-CoV-2 in a lateral flow antigen test. Despite these high viral loads, viable virus could not be cultured from wastewater after multiple attempts.

The extremely high levels of viral RNA in Facility Line B allowed us to amplify and sequence entire viral genomes to shed further light on the origins and evolution of this unusual lineage. We generated whole genome sequences from the Facility Line B samples taken on Jun. 16, Aug. 16, Sep. 23 and Sep. 27, 2022. All of these sequences were classified as lineage B.1.234 by Pangolin. In SARS-CoV-2 genomic surveillance using clinical specimens, B.1.234 viruses were first detected in Wisconsin on 2 Sep. 2020, and were last detected on Mar. 30 2021.²² Combining our observations, we posit that the simplest explanation for the appearance and persistence of the Wisconsin Lineage is that a single individual, originally infected when B.1.234 was in circulation, developed a persistent infection and continued to excrete viruses into wastewater throughout 2022.

While the original B.1.234 lineage does not have any characteristic spike RBD amino acid changes relative to the reference Wuhan-Hu-1, Omicron lineages detected in wastewater concurrently with the Wisconsin Lineage had many (FIG. 15a). RBD amplicon sequencing of the Wisconsin Lineage using non-Omicron PCR amplification detected 29 RBD changes at a frequency of at least 25%, and 43 more at a frequency of at least 10%. Sequencing single amplicons that span the RBD allowed us to define haplotypes, i.e., specific combinations of mutations found together in a single RNA molecule. We repeatedly sequenced spike RBD in wastewater samples from the Sub-District 5 interceptor line, and haplotypes of the Wisconsin Lineage were detected every month from January 2022 to January 2023 (FIG. 15b, 18). In all, we detected 87 RBD haplotypes between January 2022 and January 2023, but the mean number of haplotypes detected at any one time point was 2 (range, 1-6) (FIG. 18). The signal became undetectable in January 2023. Of the RDB amino acid changes with a frequency of at least 25%, 11 of these were at the same site as Omicron RBD changes, 9 were identical to Omicron, and 9 are absent from known Omicron lineages to date (FIG. 15a,b). Some of these exact amino acid changes, or different changes at the same positions, were initially detected in the Wisconsin Lineage months before becoming predominant in globally circulating Omicron lineages (FIG. 16).

The cryptic lineage is also highly divergent outside of the spike RBD. When plotted on a radial phylogenetic tree using Nextclade, our Illumina whole genome consensus sequences from Facility Line B show a similar degree of divergence from the Wuhan-Hu-1 reference to 22B Glade and XBB* Omicron lineages (FIG. 19). To investigate this further, we used iVar within the nf-core/viralrecon workflow to call variants at ≥25% frequency from the Wuhan-Hu-1 reference and turned that output into a pivot table (disclosed in Shafer et al Medrxiv 10.28.22281553). From this pivot table, we identified which amino acid sites had variants detected at every time point in at least one whole genome sequence replicate. One of the Wisconsin Lineage's most characteristic (and peculiar) mutations is in the N-terminal domain (NTD) of the membrane protein, where there is a 15 nucleotide insertion (I8delinsSNNSEF) found at an average frequency of 92.4% in all sequences (disclosed in Shafer et al Medrxiv 10.28.22281553).

We next asked whether the unusual combinations of mutations present in the Wisconsin Lineage could be the result of natural selection favoring nonsynonymous (amino-acid-changing) mutations. First, we calculated the nucleotide substitution rate that prevailed when B.1.234 viruses were circulating in the US Midwest (Illinois, Indiana, Iowa, Kansas, Michigan, Minnesota, Missouri, Nebraska, North Dakota, Ohio, South Dakota, and Wisconsin) and found that mutations accumulated in the Wisconsin Lineage faster than expected based on this rate (FIG. 17a). Across the four timepoints with available genome sequences, there is a notable excess of nonsynonymous nucleotide substitutions (mean 121.8±16.3) relative to synonymous ones (mean 22.5±4.7) (FIG. 17b). Some previous reports have suggested that mutations mediated by APOBEC cytidine deaminases can lead to a dramatic excess of C-to-T substitutions within SARS-CoV-2-infected hosts.2314 In our sequences, C to T transitions were the most common mutation type, but did not dramatically outnumber other types (FIG. 17c). To further characterize genetic diversity within each sample, we used the summary statistic π, which quantifies the number of pairwise differences per nonsynonymous (πN) and synonymous (πS) site within a set of sequences. Within the spike gene, πN was significantly greater than πS at each timepoint, which could indicate ongoing diversifying selection on spike (FIG. 17d). Spike also had significantly higher nonsynonymous diversity compared to ORF1ab, ORF3a, M, ORF6, ORF7a, and N at each timepoint (FIG. 17d). Because π counts pairwise differences per site within a sample, mutations that have become fixed or nearly fixed within the virus population do not contribute to π values. We therefore next calculated divergence, i.e., the average Hamming distance between each sequenced virus (either B.1.234 variants or the Wisconsin Lineages) and the ancestral sequence Wuhan-Hu-1 (MN908947.3; FIG. 17e). Both synonymous and nonsynonymous divergence values were substantially higher for the Wisconsin Lineage than for B.1.234 viruses. Notably, nonsynonymous divergence was also dramatically elevated relative to synonymous divergence in the wastewater lineage. Together these observations suggest that the accumulation of mutations in the Wisconsin Lineage, particularly in spike, is the result of adaptive evolution.

### Discussion

Here we traced the source of a cryptic SARS-CoV-2 lineage, first detected in wastewater from a central municipal wastewater treatment facility, to a single point source. At the identified point source of this cryptic lineage (Facility Line B), non-human animal sequences made up a minimal proportion of rRNA detected. Thus, the likelihood that this virus was being shed by an otherwise scarce animal reservoir is low, especially given the high viral RNA concentrations of samples from this site. We conclude that the Wisconsin Lineage, the cryptic SARS-CoV-2 lineage we detected in wastewater throughout 2022, was most likely derived from a single human individual with an unusually prolonged infection.

We detected remarkably high levels of SARS-CoV-2 RNA in Facility Line B, the source of the Wisconsin Lineage signal. In one Canadian study, the highest total viral concentrations in municipal wastewater of the three cities tested during the Omicron surge was 3.4 million gene copies/L.²⁵

Another study reported peak SARS-CoV-2 concentrations of 1.1 million copies/L coming from a single university residence hall with 328 residents.²⁶ Average N1/N2 SARS-CoV-2 concentrations detected from Facility Line B in this study peaked at 2.7 billion copies/L. This finding may help to resolve a paradox from earlier cryptic lineage studies: if cryptic lineages come from only a single source, how could they be detected in a dilute municipal wastewater sample? Based on wastewater flow data from the Sub-District 5 interceptor line and estimations of typical toilet use, we would expect the Wisconsin Lineage viral RNA to be diluted from a wastewater volume of approximately 200 gallons at Facility Line B into a volume of 8 million gallons at the Sub-District 5 interceptor. Thus, if there were 2 billion copies/L at Facility Line B, we would expect to detect ˜50,000 copies/L at the Sub-District 5 interceptor. This is a comparable concentration, if a little lower, than what we actually observed over 13 months (FIG. 14c). We hypothesize that these high levels of viral RNA result from a prolonged infection involving virus replication in the GI and/or urinary tracts, though the extent to which such infections shed more virus into the wastewater than infections where replication primarily occurs in the upper and/or lower respiratory tracts needs to be investigated further.

The large preponderance of nonsynonymous substitutions in the Facility Line B viral genomes suggests that this virus has undergone diversifying selection on spike, and perhaps on other genes. This is consistent with reports of individuals with prolonged SARS-CoV-2 infections, in whom weak immunity and persistent virus replication result in the selection of immune escape variants.^{17,18} Many RBD amino acid changes present in the Wisconsin Lineage have eventually appeared in Omicron variants circulating in human populations. In the RBD region of the spike gene, R346T, V445P, L452Q, L452R, N460K, and F486V and F486P emerged in circulating Omicron variants globally between January of 2022 and January of 2023 (FIG. 15). Some of these spike mutations, specifically R346T, V445P, and N460K, emerged in the Wisconsin Lineage five to six months before becoming highly prevalent globally (largely associated with the spread of BQ.1.1* and XBB.1.5). In the Wisconsin Lineage, a phenylalanine-to-alanine substitution at spike residue 486 (F486A) appeared approximately four months before the rise of S:F486V (found in BA.5*/BQ.1* variants) and ten months before the rise of S:F486P (found in XBB.1.5*). The RBD mutations Y453F and V483A have been detected since January and February 2022, respectively, in the Wisconsin Lineage but have been found in less than one percent of global sequences during that same time (FIG. 16). We could therefore speculate that those two substitutions, or other mutations at these sites, may become more prevalent in circulating viruses in the future.

In addition to the highly divergent spike, there was a cluster of fixed variants in the region that encodes the ectodomain of the viral membrane protein. The mutation cluster includes a 15-nucleotide insertion (5′-GCAACAACTCAGAGT-3′ SEQ ID NO: 28) that encodes the amino acids SNNSEF (SEQ ID NO: 31) by splitting the A and TT of an existing isoleucine codon. Interestingly, the insertion is identical to the sequence found between positions 11,893 and 11,907 in ORF1ab which suggests intramolecular recombination. Additionally, the Wisconsin Lineage has M:A2E, M:G6C, and M:L17V amino acid substitutions. The phenotypic impact of these substitutions, if any, is unclear. One possible explanation is diversifying selection of immune escape variants. This region of the membrane protein is exposed outside of the SARS-CoV-2 virion and is a known target for binding antibodies.^{27,28} In Heffron et al., 2021, membrane-binding antibodies were present at a higher level than spike-binding antibodies.

Our data strongly suggest that SARS-CoV-2 cryptic lineages in wastewater originate from human sources. While animal sources may contribute in other settings, that is not the case here. This has several important implications. Such lineages likely exist wherever people are infected with SARS-CoV-2, i.e., worldwide. That is, many, perhaps most, divergent SARS-CoV-2 lineages detected in wastewater likely reflect ongoing human infections, and may therefore pose a transmission risk to others. The elevated number of nonsynonymous substitutions in the wastewater variant, and its accumulation of mutations at a rate faster than expected based on its ancestral lineage, resemble attributes of the original Omicron lineage when it emerged.²⁹ Indeed, a leading hypothesis for the origin of many previous SARS-CoV-2 variants of concern is that they arose in immunocompromised individuals with prolonged infections.^{17,18} The fact that the Wisconsin Lineage appears to be derived from a prolonged infection with an ancestral B.1.234 virus further highlights the importance of prolonged infections in the emergence of highly divergent viruses and emphasizes the importance of identifying, tracing, and treating such infections.

To this end, more frequent global wastewater viral surveillance/sequencing of catchment areas would likely detect many more examples of cryptic SARS-CoV-2 lineages. We speculate that Omicron derived cryptic lineages will be detectable in wastewater in the future. Given the extensive spread of Omicron, the number of prolonged infections that give rise to these cryptic lineages is also expected to increase, making the emergence and detection of cryptic lineages more common. Although RBD sequencing covers only a small segment of the SARS-CoV-2 genome, we believe this method will continue to be valuable in wastewater surveillance due to its high sensitivity. We note that individuals with immunocompromising conditions are at increased risk for prolonged infections but may not be the only population in which such infections occur; the facility in which the cryptic lineage was detected was an otherwise unremarkable business, not a healthcare facility or other location with medically fragile occupants. SARS-CoV-2 cryptic lineage sequences could aid in forecasting the future evolutionary trajectory of SARS-CoV-2 to evaluate the cross-protection of existing and future vaccines and monoclonal antibodies. In the present, wastewater surveillance has become an irreplaceable window into the progression of the pandemic as clinical sampling wanes and more human-derived cryptic wastewater lineages await detection.

### References

1. Xiao F, Tang M, Zheng X, Liu Y, Li X, Shan H. Evidence for Gastrointestinal Infection of SARS-CoV-2. Gastroenterology 05/2020; 158: 1831-3.e3.
2. Anjos D, Fiaccadori F S, Servian C do P, et al. SARS-CoV-2 loads in urine, sera and stool specimens in association with clinical features of COVID-19 patients. Journal of Clinical Virology Plus 02/2022; 2: 100059.
3. Ahmed W, Tscharke B, Bertsch P M, et al. SARS-CoV-2 RNA monitoring in wastewater as a potential early warning system for COVID-19 transmission in the community: A temporal case study. Sci Total Environ 03/2021; 761: 144216.
4. Gregory D A, Trujillo M, Rushford C, et al. Genetic Diversity and Evolutionary Convergence of Cryptic SARS-CoV-2 Lineages Detected Via Wastewater Sequencing. medRxiv : the preprint server for health sciences 2022; published online June 3. DOI:10.1101/2022.06.03.22275961.
5. Lan J, Ge J, Yu J, et al. Structure of the SARS-CoV-2 spike receptor-binding domain bound to the ACE2 receptor. Nature 2020; 581: 215-20.
6. Abdool Karim S S, Karim Q A. Omicron SARS-CoV-2 variant: a new chapter in the COVID-19 pandemic. Lancet 2021; 398: 2126.
7. Coy-Lineages. https://cov-lineages.org/global_report_BA.1.html (accessed Jul. 27, 2022).
8. Fritz M, Rosolen B, Krafft E, et al. High prevalence of SARS-CoV-2 antibodies in pets from COVID-19+ households. One Health 2021; 11. DOI:10.1016/j .onehlt.2020.100192.
9. Hale V L, Dennis P M, McBride D S, et al. SARS-CoV-2 infection in free-ranging white-tailed deer. Nature 2022; 602: 481-6.
10. Chandler J C, Bevins S N, Ellis J W, et al. SARS-CoV-2 exposure in wild white-tailed deer (Odocoileus virginianus). Proc Natl Acad Sci U S A 2021; 118. D01:10.1073/pnas.2114828118.
11. Pickering B, Lung O, Maguire F, et al. Divergent SARS-CoV-2 variant emerges in white-tailed deer with deer-to-human transmission. Nat Microbiol 2022; 7: 2011-24.

12. Rasmussen T B, Fonager J, Jørgensen C S, et al. Infection, recovery and re-infection of farmed mink with SARS-CoV-2. PLoS Pathog 2021; 17: e1010068.

13. Lu L, Sikkema R S, Velkers FC, et al. Adaptation, spread and transmission of SARS-CoV-2 in farmed minks and associated humans in the Netherlands. Nat Commun 2021; 12: 6802.
14. Bb O M, Sikkema R S, Nieuwenhuijse D F, et al. Transmission of SARS-CoV-2 on mink farms between humans and mink and back to humans. Science 2021; 371. DOI:10.1126/science.abe5901.
15. Domańska-Blicharz K, Oude Munnink B B, Orlowska A, et al. Cryptic SARS-CoV-2 lineage identified on two mink farms as a possible result of long-term undetected circulation in an unknown animal reservoir, Poland, November 2022 to January xs2023. Euro Surveill 2023; 28. DOI:10.2807/1560-7917. ES.2023.28.16.2300188.
16. Pérez-Cataluña A, Chiner-Oms Á, Cuevas-Ferrando E, et al. Spatial and temporal distribution of SARS-CoV-2 diversity circulating in wastewater. Water Res 2022; 211: 118007.
17. Corey L, Beyrer C, Cohen M S, Michael N L, Bedford T, Rolland M. SARS-CoV-2 Variants in Patients with Immunosuppression. N Engl J Med 2021; 385: 562-6.
18. Wilkinson S A J, Richter A, Casey A, et al. Recurrent SARS-CoV-2 mutations in immunodeficient patients. Virus Evol 2022; 8: veac050.
19. Gregory D A, Wieberg CG, Wenzel J, Lin C-H, Johnson M C. Monitoring SARS-CoV-2 Populations in Wastewater by Amplicon Sequencing and Using the Novel Program SAM Refiner. Viruses 2021; 13: 1647.
20. Ewels P A, Peltzer A, Fillinger S, et al. The nf-core framework for community-curated bioinformatics pipelines. Nat Biotechnol 2020; 38: 276-8.
21. Karthikeyan S, Levy J I, De Hoff P, et al. Wastewater sequencing reveals early cryptic SARS-CoV-2 variant transmission. Nature 2022; 609: 101-8.
22. outbreak.info. outbreak.info. (accessed Sep. 8, 2022).
23. Di Giorgio S, Martignano F, Torcia M G, Mattiuz G, Conticello S G. Evidence for host-dependent RNA editing in the transcriptome of SARS-CoV-2. Sci Adv 2020; 6: eabb5813.
24. Liu X, Liu X, Zhou J, Dong Y, Jiang W, Jiang W. Rampant C-to-U deamination accounts for the intrinsically high mutation rate in SARS-CoV-2 spike gene. RNA 2022; 28: 917-26.
25. Oloye F F, Xie Y, Asadi M, et al. Rapid transition between SARS-CoV-2 variants of concern Delta and Omicron detected by monitoring municipal wastewater from three Canadian cities. Sci Total Environ 2022; 841: 156741.
26. Schmitz B W, Innes G K, Prasek S M, et al. Enumerating asymptomatic COVID-19 cases and estimating SARS-CoV-2 fecal shedding rates via wastewater-based epidemiology. Sci Total Environ 2021; 801: 149794.
27. Jörrißen P, Schütz P, Weiand M, et al. Antibody Response to SARS-CoV-2 Membrane Protein in Patients of the Acute and Convalescent Phase of COVID-19. Front Immunol 2021; 0. DOI: 10.3389/fimmu.2021. 679841.
28. Heffron A S, McIlwain S J, Amjadi M F, et al. The landscape of antibody binding in SARS-CoV-2 infection. bioRxiv. DOI:10.1101/2020.10.10.334292.
29. Moulana A, Dupic T, Phillips A M, et al. Compensatory epistasis maintains ACE2 affinity in SARS-CoV-2 Omicron BA.1. Nat Commun 2022; 13: 7011.
30. Chen C, Nadeau S, Yared M, et al. CoV-Spectrum: analysis of globally shared SARS-CoV-2 data to identify and characterize new variants. Bioinformatics 2022; 38: 1735-7.

### Example IV: Wastewater Cryptic Variants Proceed Globally Circulating Variants

The inventors propose that evolutionarily advanced variants detected in wastewater (EACL; evolutionarily advanced cryptic lineages) and patient sequences (EAPL; evolutionarily advanced patient lineages) precede globally circulating ones, an assertion backed by continued work on the Wisconsin EACL, whose Spike variants anticipated global SARS-CoV-2 Omicron variants (FIG. 16). The dates at the bottom of the panel indicate when these variants were detected in the EACL, while the top panel shows the frequency of variants at these sites in globally circulating sequences.

The inventors successfully identified EACL and EAPL via NCBI SRA data mining. A new EACL found in two sewersheds around 40 miles apart in Ohio (SRA records SRR24054801 and SRR24054763) further support the hypothesis that single individuals are shedding EACL.

Moreover, the inventors found nearly early identical EACL (SRA ERR10058890) and EAPL (GISAID EPI_ISL_5280146; SRA ERX6769989) in Liverpool, UK in October 2021, linking wastewater EACL detection to nasal swab EAPL detection. This suggests some individuals exhale viruses with EACL-like sequences, motivating continued study of both EACL and EAPL.

FIG. 20 demonstrates the Ohio, Wisconsin, and UK EACL/EAPL genome sequences are about as divergent from the original SARS-CoV-2 as Omicron viruses (clades 21M,K,L, 22*, and 23*). However, the EACL/EAPL evolved these extensive changes independently of each other, most likely during prolonged infections.

FIG. 21 reveals convergent Spike amino acid variants in the Wisconsin, Ohio, and UK EACL/EAPL, implying shared evolutionary pressures. Variant residues shared between at least two EACL/EAPL or all four are highlighted in light and dark blue, respectively. Variants in circulating viruses in the UK and US when these EACL/EAPL were detected are shown on the right. Despite their independent evolution, some of the EACL/EAPL evolutionary pressures (e.g., escape from antibody responses directed against Spike) result in the selection of remarkably similar variants. This convergence can be harnessed to forecast residues, like Spike V367 and L828, and specific amino acid variants that we predict will eventually increase in frequency in globally circulating SARS-CoV-2 viruses.

Discovering more EACL and EAPL will enhance our understanding of virus features that best predict future SARS-CoV-2 variation and allow better characterization of the risk posed by these enigmatic viruses for transmission between individuals in the future.

We claim:

1. A method for identifying antigenic variants from cryptic lineages arising in a virus population comprising:
collecting a urine or stool sample from a subject with a prolonged infection or from wastewater samples from at least one location;
extracting RNA from the sample;
sequencing the variable regions of viral RNA from wastewater; and
identifying cryptic lineages containing antigenic variants in the virus population.

2. The method of claim 1, wherein the virus is SARS-CoV-2.

3. The method of claim 2, wherein the antigen is the Spike or membrane protein.

4. The method of claim 3, wherein the variable region is the RBD of the Spike protein or the first 19 amino acids of the membrane protein.

5. The method of claim 1, further comprising testing recombinant virus comprising the antigenic variants in an antibody neutralization assay.

6. The method of claim 5, further comprising generating antibodies capable of recognizing the antigenic variants.

7. The method of claim 6, wherein the antibodies are neutralizing antibodies and block replication of a virus comprising the antigenic variant.

8. The method of claim 6, wherein the antibodies are monoclonal antibodies.

9. The method of claim 1, further comprising generating a vaccine comprising at least one of the antigenic variants.

10. The method of claim 9, wherein the vaccine is a mRNA, peptide, or inactivated viral vaccine.

11. The method of claim 9, wherein the vaccine comprises more than one antigenic variant.

12. The method of claim 1, wherein the variable regions are amplified with RT-PCR primers comprising SEQ ID NO: 6 or 16 and 7 or SEQ ID NO: 25 and 26 prior to the sequencing step.

13. The method of claim 12, wherein the RT-PCR primers are SEQ ID NO: 6 and 7 and a second amplification is completed prior to sequencing using a set of nested primers of SEQ ID NO: 8 and 9.

14. The method of claim 12, wherein the RT-PCR primers are SEQ ID NO: 25 and 26 and a second amplification is completed prior to the sequencing using a set of nested primers of SEQ ID NO: 8 and 18.

15. The method of claim 12, wherein the RT-PCR primers amplify at least a 1.5 kb region encoding a SARS-CoV-2 Spike protein.

16. The method of claim 15, wherein the RT-PCR primers are
(SEQ ID NO: 16)
CCCTGCATACACTAATTCTTTCAC
and
(SEQ ID NO: 7)
TCCTGATAAAGAACAGCAACCT.

17. The method of claim 16, further comprising a nested amplification step, wherein the nested primers are CATTCAACTCAGGACTTGTTCTT (SEQ ID NO: 17) and ATGTCAAGAATCTCAAGTGTCTG (SEQ ID NO: 18).

18. The method of claim 1, further comprising analyzing the cryptic lineages containing antigenic variants and determining if the antigenic variants become variants of concern.

19. The method of claim 1, wherein databases with viral sequences from wastewater or databases with sequences from virally infected subjects are used to identify cryptic lineages containing antigenic variants in the virus population.

20. The method of claim 1, wherein the RNA is extracted from fecal matter or urine.


SEQUENCE LISTING (31 sequences)

SEQ ID NO: 1 - DNA, 20 bp, synthetic construct
  source 1..20: mol_type = other DNA
gaccccaaaatcagcgaaat

SEQ ID NO: 2 - DNA, 24 bp, synthetic construct
  source 1..24: mol_type = other DNA
tctggttactgccagttgaatctg

SEQ ID NO: 3 - skipped

SEQ ID NO: 4 - DNA, 55 bp, synthetic construct
  source 1..55: mol_type = other DNA
tcgtcggcagcgtcagatgtgtataagagacagccagatgattttacaggctgcg

SEQ ID NO: 5 - DNA, 61 bp, synthetic construct
  source 1..61: mol_type = other DNA
gtctcgtgggctcggagatgtgtataagagacaggaaagtactactactctgtatggttgg

SEQ ID NO: 6 - DNA, 27 bp, synthetic construct
  source 1..27: mol_type = other DNA
ctgctttactaatgtctatgcagattc

SEQ ID NO: 7 - DNA, 22 bp, synthetic construct
  source 1..22: mol_type = other DNA
tcctgataaagaacagcaacct

SEQ ID NO: 8 - DNA, 55 bp, synthetic construct
  source 1..55: mol_type = other DNA
acactctttccctacacgacgctcttccgatctgtgatgaagtcagacaaatcgc

SEQ ID NO: 9 - DNA, 57 bp, synthetic construct
  source 1..57: mol_type = other DNA
gtgactggagttcagacgtgtgctcttccgatctatgtcaagaatctcaagtgtctg

SEQ ID NO: 10 - DNA, 51 bp, synthetic construct
  source 1..51: mol_type = other DNA
tcgtcggcagcgtcagatgtgtataagagacagactgggattagatacccc

SEQ ID NO: 11 - DNA, 51 bp, synthetic construct
  source 1..51: mol_type = other DNA
gtctcgtgggctcggagatgtgtataagagacagagaacaggctcctctag

SEQ ID NO: 12 - DNA, 51 bp, synthetic construct
  source 1..51: mol_type = other DNA
acactctttccctacacgacgctcttccgatctactgggattagatacccc

SEQ ID NO: 13 - DNA, 52 bp, synthetic construct
  source 1..52: mol_type = other DNA
gtgactggagttcagacgtgtgctcttccgatcttagaacaggctcctctag

SEQ ID NO: 14 - DNA, 22 bp, synthetic construct
  source 1..22: mol_type = other DNA
ncctgataaagaacagcaacct

SEQ ID NO: 15 - DNA, 55 bp, synthetic construct
  source 1..55: mol_type = other DNA
acactctttccctacacgacgctcttccgatctgtratgaagtcagmcaaatygc

SEQ ID NO: 16 - DNA, 24 bp, synthetic construct
  source 1..24: mol_type = other DNA
ccctgcatacactaattctttcac

SEQ ID NO: 17 - DNA, 23 bp, synthetic construct
  source 1..23: mol_type = other DNA
cattcaactcaggacttgttctt

SEQ ID NO: 18 - DNA, 23 bp, synthetic construct
  source 1..23: mol_type = other DNA
atgtcaagaatctcaagtgtctg

SEQ ID NO: 19 - DNA, 22 bp, synthetic construct
  source 1..22: mol_type = other DNA
attcaactcaggacttgttctt

SEQ ID NO: 20 - DNA, 20 bp, synthetic construct
  source 1..20: mol_type = other DNA
atacaaaccacgccaggtag

SEQ ID NO: 21 - DNA, 21 bp, synthetic construct
  source 1..21: mol_type = other DNA
aacccttagacacagcaaagt

SEQ ID NO: 22 - DNA, 55 bp, synthetic construct
  source 1..55: mol_type = other DNA
acactctttccctacacgacgctcttccgatctggtagtggagttcctgttgtag

SEQ ID NO: 23 - DNA, 54 bp, synthetic construct
  source 1..54: mol_type = other DNA
gtgactggagttcagacgtgtgctcttccgatctagcacgtagtgcgtttatct

SEQ ID NO: 24 - DNA, 24 bp, synthetic construct
  source 1..24: mol_type = other DNA
attctgtcctatataattccgcat

SEQ ID NO: 25 - DNA, 22 bp, synthetic construct
  source 1..22: mol_type = other DNA
ccctgataaagaacagcaacct

SEQ ID NO: 26 - DNA, 25 bp, synthetic construct
  source 1..25: mol_type = other DNA
tatataattccgcatcattttccac

SEQ ID NO: 27 - DNA, 55 bp, synthetic construct; identical to SEQ ID NO: 8
  source 1..55: mol_type = other DNA

SEQ ID NO: 28 - DNA, 15 bp, Severe acute respiratory syndrome-related coronavirus
  source 1..15: mol_type = genomic DNA
gcaacaactcagagt

SEQ ID NO: 29 - DNA, 32 bp, Severe acute respiratory syndrome-related coronavirus
  source 1..32: mol_type = genomic DNA
tctctacagtgttcccacttacaagttttgga

SEQ ID NO: 30 - DNA, 32 bp, Severe acute respiratory syndrome-related coronavirus
  source 1..32: mol_type = genomic DNA
tctctacagtgttcccacctacaagttttgga

SEQ ID NO: 31 - protein, 6 aa, synthetic construct
SNNSEF